(19) United States
(12) Patent Application Publication
(10) Pub. No.: US 2006/0276477 A1
(43) Pub. Date: Dec. 7, 2006

(54) Treatment method for anemia

(75) Inventors: Stephen J. Klaus, San Francisco, CA (US); Thomas B. Neff, Atherton, CA (US)

Correspondence Address: FIBROGEN, INC.;INTELLECTUAL PROPERTY DEPARTMENT, 225 GATEWAY BOULEVARD, SOUTH SAN FRANCISCO, CA, 94080, US

(73) Assignee: FibroGen, Inc., South San Francisco, CA (US)

(21) Appl. No.: 11/448,326

(22) Filed: Jun. 6, 2006

Related U.S. Application Data
(60) Provisional application No. 60/688,161, filed on Jun. 6, 2005.

(51) Int. Cl. A61K 31/502; A61K 31/4745; A61K 31/4704

(52) U.S. Cl. 514/248; 514/312; 514/291; 514/303

(57) ABSTRACT

The present invention relates to improved methods for treating anemia. Methods and compounds useful for treating anemia, wherein the anemia treatment is associated with a lower risk of thrombosis or hypertension compared to that observed with rhEPO therapy, are provided.

This application claims the benefit of U.S. Provisional Application Serial Number 60/688,161, filed on 6 Jun. 2005, incorporated by reference herein in its entirety.

### FIELD OF THE INVENTION

The present invention relates to improved methods for treating anemia. Methods and compounds useful for treating anemia, wherein the anemia treatment is associated with a lower risk of thrombosis or hypertension compared to that observed with rhEPO therapy, are provided.

### BACKGROUND

Current therapy for anemia (including conditions such as anemia associated with kidney disease, e.g. end-stage renal disease; anemia of chronic disease; anemia of cancer; chemotherapy-induced anemia; iron deficiency anemia etc.) is administration of erythropoiesis stimulating proteins (ESPs), such as recombinant human erythropoietin (rhEPO) and Aranesp (Amgen; Thousand Oaks, Calif.).

However, the use of recombinant human EPO therapy is associated with various risks. Increased morbidity and mortality have been associated with administration of higher doses of rhEPO in patients that are resistant to rhEPO (Zhang et al, 2004 Am J Kidney Disease 44:866-876). Furthermore, administration of rhEPO is associated with an increase in thrombosis risk and hypertension risk. This is often attributed to changes in red cell mass affecting blood viscosity and rheology, but the underlying mechanism(s) operative have not been definitively demonstrated.

Clinical trials have suggested that a measurable increase in thrombotic complications occurs in patients treated with rhEPO (Wun T, Law L, Harvey D, Sieracki B, Scudder S A, Ryu J K. Increased incidence of symptomatic venous thrombosis in patients with cervical carcinoma treated with concurrent chemotherapy, radiation, and erythropoietin. Cancer 2003;98(7):1514-20). In 2004, the Food and Drug Administration's Oncology Drug Advisory Committee (FDA ODAC) met to consider the safety of ESPs in oncology. Sponsors of such agents for use in the treatment of chemotherapy-induced anemia presented analyses of their safety data, including meta-analyses of thrombosis in all placebo controlled trials (see fda.gov/ohrms/dockets/ac/04/briefing/4037b2.htm). These meta-analyses indicated an increased risk of non-fatal thrombotic complications associated with ESP use. The risk in both the placebo and ESP groups was higher for patients with a history of thrombosis. The overall impact on thrombosis risk has been confirmed in an independent meta-analysis of rhEPO data (Bohlius J, Langensiepen S, Schwarzer G, et al. Recombinant Human Erythropoietin and Overall Survival in Cancer Patients: Results of a Comprehensive Meta-analysis. Journal of the National Cancer Institute 2005;97(7):490-8).

The datasets considered by the FDA ODAC did not agree on whether thrombosis risk correlated with baseline or peak hemoglobin concentration or with the rate of rise in this parameter. There are several alternative, more plausible, mechanisms that could explain an increased thrombosis risk associated with ESP therapy, independent of an effect on blood viscosity and rheology, including: direct activation of vascular endothelium and platelets (Stohlawetz P J, Dzirlo L, Hergovich N, et al. Effects of erythropoietin on platelet reactivity and thrombopoiesis in humans. Blood 2000;95(9):2983-9), activation of platelets by young erythrocytes (Valles J, Santos M T, Aznar J, et al. Erythrocytes metabolically enhance collagen-induced platelet responsiveness via increased thromboxane production, adenosine diphosphate release, and recruitment. Blood 1991;78(1):154-62; Valles J, Santos M T, Aznar J, et al. Platelet-erythrocyte interactions enhance alpha(IIb)beta(3) integrin receptor activation and P-selectin expression during platelet recruitment: down-regulation by aspirin ex vivo. Blood 2002;99(11):3978-84), and synergy with thrombopoietin in the activation of platelets (Wun T, Paglieroni T, Hammond W P, Kaushansky K, Foster D C. Thrombopoietin is synergistic with other hematopoietic growth factors and physiologic platelet agonists for platelet activation in vitro. Am J Hematol 1997;54(3):225-32).

There is thus a need for methods for treating anemia, which methods do not carry an associated risk for thrombosis or thrombotic complications, or for hypertension. There is a need for effective treatments for anemia in EPO-resistant subjects, subjects in which rhEPO therapy has been associated with increased risk of morbidity and mortality.

### SUMMARY OF THE INVENTION

The present invention provides methods that are effective for treating anemia, but which lead to only small increases in the levels of circulating EPO. It appears that the hemoglobin levels achievable using the methods of the invention increase hemoglobin levels usefully, but do not elevate circulating EPO to levels that are associated with problematic complications. This is beneficial as current methods of treating anemia, by administration of rhEPO or other ESPs, result in ‘high’ or ‘elevated’ circulating EPO levels, which is associated with various risks, including increased mortality and increased risk of thrombotic complications.

The present invention also provides methods for treating anemia or increasing hemoglobin levels in a subject, wherein the anemia treatment or the increased hemoglobin levels are associated with a lower risk of thrombosis or hypertension compared to that observed with rhEPO therapy.

Current guidelines relating to rhEPO administration define target hemoglobin levels for an adult subject as 12 gm/dL, corresponding to a hematocrit of 36%. These guidelines reflect the concern that the amount of rhEPO that would be administered to a subject to reach higher hemoglobin levels, for example, levels above 12 gm/dL, would be an amount associated with a greatly increased risk for development of thrombosis or of thrombotic complications, and for development of hypertension. Further, such amounts of rhEPO would be prohibitively expensive. Therefore, the present invention provides methods in which hemoglobin levels in a subject are increased to a level of about 12 gm/dL. Methods for increasing hematocrit to about 36% are also provided. Methods of increasing hemoglobin levels to above 10 gm/dL, above 11 gm/dL, above 12 gm/dL, above 13 gm/dL, and above 14 gm/dL are also contemplated, as are methods for raising hematocrit to above 30%, above 33%, above 36%, above 39%, and above 42%, respectively. Hemoglobin levels and hematocrit of this magnitude, as raised according to the methods of the invention, are associated with a lower risk of thrombosis compared to that observed with rhEPO therapy. Furthermore, these hemoglobin levels are associated with a lower risk of hypertension compared to that observed with rhEPO therapy.

The present invention also provides methods for treating anemia or increasing hematocrit in a subject, wherein the anemia treatment or the increased hematocrit are associated with a lower risk of thrombosis or hypertension compared to that observed with rhEPO therapy.

These methods are effected by administering an agent that stabilizes HIFα. Preferably, the agent is a compound that inhibits HIF prolyl hydroxylase activity.

Accordingly, in one embodiment, the present invention provides a method for treating a subject having anemia, said method comprising administering to the subject an effective amount of an agent that stabilizes HIFα. The present invention also provides for the use of an agent that stabilizes HIFα in the manufacture of a medicament for the treatment of anemia. Preferably, the agent is a compound that inhibits HIF prolyl hydroxylase activity.

Preferably, administration of an agent of the present invention to a subject results in an increase in the circulating level of EPO in that subject to a level in the range of 10-1000 mIU/ml (assuming a basal endogenous level of 10 mIU/ml). In some embodiments, the level is raised to a level in the range of 10-500 mIU/ml, 10-400 mIU/ml, 10-300 mIU/ml, 10-200 mIU/ml, 10-150 mIU/ml, 10-100 mIU/ml, 10-90 mIU/ml, 10-80 mIU/ml, 10-70 mIU/ml, 10-60 mIU/ml, 10-50 mIU/ml, 10-40 mIU/ml, 10-30 mIU/ml, 10-20 mIU/ml, or 10-15 mIU/ml. More preferably, it is raised to a level in the range of 10-100 mIU/ml, 10-75 mIU/ml, 10-50 mIU/ml, 10-25 mIU/ml, or 10-15 mIU/ml. More preferably still, it is raised to a level in the range of 10-50 mIU/mi, 10-45 mIU/ml, 1040 mIU/ml, 10-35 mIU/ml, 10-30 mIU/ml,-10-25 mIU/ml, 10-20 mIU/ml. or 10-15 mIU/ml.

In contrast, administration of therapeutically effective doses of rhEPO to the subject results in a greater increase in the circulating level of EPO, for example to a level in the range of 100 to 20000 mIU/ml, levels that have been associated with development of thrombosis and thrombotic complications, development of hypertension, etc.

Preferably, administration of an agent of the present invention to a subject results in an increase in baseline hemoglobin level in that subject by a level in the range of 0.1-5.0 g/dL. In some embodiments, the level is increased by a level in the range of 0.2-5.0 g/dL., 0.5-5.0 g/d., 1.0-5.0 g/d., 1.5-5.0 g/dL, 2.0-5.0 g/dL, 3.0-5.0 g/dL, or 4.0-5.0 g/dL. More preferably, the level is increased by an amount in the range of 0.2-2.5 g/dL, 0.4-2.5 g/dL, 0.6-2.5 g/dL, 0.8-2.5 g/dL, 1.0-2.5 g/dL, 1.2-2/5 g/dL, 1.4-2.5 g/dL, 1.6-2.5 g/dL, 1.8-2.5 g/dL, or 2-2.5 g/dL. More preferably still, it is raised to a level in the range of 1.0-2.0 g/dL, 1.1-2.0 g/dL, 1.2-2.0 g/dL, 1.3-2.0 g/dL, 1.4-2.0 g/dL, 1.5-2.0 g/dL, 1.6-2.0 g/dL, 1.7-2.0 g/dL, 1.8-2.0 g/dL, or 1.9-2.0 g/dL.

The agent used in the present methods can be any agent that inhibits HIF hydroxylase activity, including, e.g., a polynucleotide, e.g., antisense sequence; a polypeptide; an antibody or fragment thereof, a small molecule, etc. A preferred agent of the present invention is a small molecule compound that inhibits HIF hydroxylase activity. In further embodiments, the agent is selected from the group consisting of 2-oxoglutarate mimetics. Agents for use in the present methods include, but are not limited to, agents of Formulae I, II, III, V, and V.

In a preferred embodiment, the agent is a 2-oxoglutarate mimetic. Such compounds may inhibit the target 2-oxoglutarate dioxygenase enzyme family member competitively with respect to 2-oxoglutarate. (Majamaa et al. (1984) Eur J Biochem 138:239-245; and Majamaa et al., supra.) In certain embodiments, the 2-oxoglutarate mimetic is selected from the group consisting of a compound of Formula I, Formula II, Formula IV, and Formula V. In particular embodiments, the 2-oxoglutarate mimetic is a pyridine-2-carboxamide including, but not limited to, various compounds of Formula I. In particular embodiments, the 2-oxoglutarate mimetic is a quinoline-2-carboxamide including, but not limited to, those of Formula Ia. In particular embodiments, the 2-oxoglutarate is an isoquinoline-3-carboxamide including, but not limited to, those of Formula Ib.

In further embodiments, an agent for use in the present methods is selected from the group consisting of: [4-Hydroxy-1-(naphthalen-2-yloxy)-isoquinoline-3-carbonyl]-amino}-acetic acid; {[4-Hydroxy-1-(pyridin-3-yloxy)-isoquinoline-3-carbonyl]-amino}-acetic acid; {[4-Hydroxy-1-(4-methoxy-phenoxy)-isoquinoline-3-carbonyl]-amino}-acetic acid; {[4-Hydroxy-1-(3-methoxy-phenoxy)-isoquinoline-3-carbonyl]-amino}-acetic acid; {[1-(3-Fluoro-phenoxy)-4-hydroxy-isoquinoline-3-carbonyl]-amino}-acetic acid; {[1-(4-Fluoro-phenoxy)-4-hydroxy-isoquinoline-3-carbonyl]-amino}-acetic acid; {[1-(2-Fluoro-phenoxy)4-hydroxy-isoquinoline-3-carbonyl]-amino}-acetic acid; {[4-Hydroxy-1-(2-methoxy-phenoxy)-isoquinoline-3-carbonyl]-amino}-acetic acid; {[1-(4-Acetylamino-phenoxy)-4-hydroxy-isoquinoline-3-carbonyl]-amino}-acetic acid; {[4-Hydroxy-1-(4-methanesulfonylamino-phenoxy)-isoquinoline-3-carbonyl]-amino}-acetic acid; [(4-Hydroxy-1-phenylamino-isoquinoline-3-carbonyl)-amino]-acetic acid; {[4-Hydroxy-6-(pyridin-3-yloxy)-isoquinoline-3-carbonyl]-amino}-acetic acid; {[4-Hydroxy-7-(pyridin-3-yloxy)-isoquinoline-3-carbonyl]-amino}-acetic acid; [(1-Chloro-4-methoxy-isoquinoline-3-carbonyl)-amino]-acetic acid; [(1-Chloro-4-ethoxy-isoquinoline-3-carbonyl)-amino]-acetic acid; [(4-Hydroxy-1-methoxy-isoquinoline-3-carbonyl)-amino]-acetic acid; [(1-Ethoxy4-hydroxy-isoquinoline-3-carbonyl)-amino]-acetic acid; [(4-Acetoxy-1-phenyl-isoquinoline-3-carbonyl)-amino]-acetic acid; [(4-Hydroxy-1-phenyl-isoquinoline-3-carbonyl)-amino]-acetic acid; [(1-Ethoxy4-phenyl-isoquinoline-3-carbonyl)-amino]-acetic acid; [(1-Chloro-4-phenyl-isoquinoline-3-carbonyl)-amino]-acetic acid; [(4-Phenyl-isoquinoline-3-carbonyl)-amino]-acetic acid; [(4-Hydroxy-1-methyl-isoquinoline-3-carbonyl)-amino]-acetic acid; [(4-Hydroxy-1-methoxymethyl-isoquinoline-3-carbonyl)-amino]-acetic acid; [(1-Dimethylcarbamoyl-4-hydroxy-isoquinoline-3-carbonyl)-amino]-acetic acid; [(4-Hydroxy-1-methyl-6-phenoxy-isoquinoline-3-carbonyl)-amino]-acetic acid; [(4-Hydroxy-1-methyl-7-phenoxy-isoquinoline-3-carbonyl)-amino]-acetic acid; [(4-Benzyloxy-1-methyl-7-phenoxy-isoquinoline-3-carbonyl)-amino]-acetic acid; [(4-Ethoxy-1-methyl-7-phenoxy-isoquinoline-3-carbonyl)-amino]-acetic acid; [(1-Dimethylcarbamoyl-4-hydroxy-7-phenoxy-isoquinoline-3-carbonyl)-amino]-acetic acid; [(4-Hydroxy-1-methoxymethyl-7-phenoxy-isoquinoline-3-carbonyl)-amino]-acetic acid; [(4-Hydroxy-1-p-tolyl-isoquinoline-3-carbonyl)-amino]-acetic acid; {[7-(4-Fluoro-phenoxy)-4-hydroxy-1-methyl-isoquinoline-3-carbonyl]-amino}-acetic acid; {[1-Chloro-4-hydroxy-7-(4-methoxy-phenoxy)-isoquinoline-3-carbonyl]-amino}-acetic acid; {[4-Hydroxy-7-(4-methoxy-phenoxy)-isoquinoline-3-carbonyl]-amino}-acetic acid; {[1-Chloro-4-hydroxy-6-(4-methoxy-phenoxy)-isoquinoline-3-carbonyl]-amino}-acetic acid; {[4-Hydroxy-6-(4-methoxy-phenoxy)-isoquinoline-3-carbonyl]-amino}-acetic acid; {[1-Chloro-4-hydroxy-7-(4-trifluoromethyl-phenoxy)-isoquinoline-3-carbonyl]-amino}-acetic acid; {[4-Hydroxy-7-(4-trifluoromethyl-phenoxy)-isoquinoline-3-carbonyl]-amino)-acetic acid; {[1-Chloro-4-hydroxy-6-(4-trifluoromethyl-phenoxy)-isoquinoline-3-carbonyl]-amino}-acetic acid; {[4-Hydroxy-6-(4-trifluoromethyl-phenoxy)-isoquinoline-3-carbonyl]-amino}-acetic acid; {[1-Chloro-7-(4-fluoro-phenoxy)-4-hydroxy-isoquinoline-3-carbonyl]-amino}-acetic acid; {[7-(4-Fluoro-phenoxy)-4-hydroxy-isoquinoline-3-carbonyl]-amino}-acetic acid; {[1-Chloro-6-(4-fluoro-phenoxy)-4-hydroxy-isoquinoline-3-carbonyl]-amino}-acetic acid; {[6-(4-Fluoro-phenoxy)-4-hydroxy-isoquinoline-3-carbonyl]-amino}-acetic acid; {[4-Hydroxy-7-(pyridin4-ylsulfanyl)-isoquinoline-3-carbonyl]-amino}-acetic acid; {[4-Hydroxy-6-(pyridin4-ylsulfanyl)-isoquinoline-3-carbonyl]-amino}-acetic acid; [(7-Benzenesulfinyl4-hydroxy-isoquinoline-3-carbonyl)-amino]-acetic acid; [(7-Benzenesulfonyl-4-hydroxy-isoquinoline-3-carbonyl)-amino]-acetic acid; [(6-Benzenesulfinyl-4-hydroxy-isoquinoline-3-carbonyl)-amino]-acetic acid; [(6-Benzenesulfonyl-4-hydroxy-isoquinoline-3-carbonyl)-amino]-acetic acid; [(6-Amino4-hydroxy-isoquinoline-3-carbonyl)-amino]-acetic acid; {[4-Hydroxy-7-(4-methoxy-benzenesulfonylamino)-isoquinoline-3-carbonyl]-amino}-acetic acid; {[4-Hydroxy-7-(3-phenyl-ureido)-isoquinoline-3-carbonyl]-amino}-acetic acid; {[4-Hydroxy-6-(3-phenyl-ureido)-isoquinoline-3-carbonyl]-amino}-acetic acid; [(4-Hydroxy-1-phenylsulfanyl-isoquinoline-3-carbonyl)-amino]-acetic acid; {[1-(4-Chloro-phenylsulfanyl)4-hydroxy-isoquinoline-3-carbonyl]-amino}-acetic acid; [(4-Hydroxy-1-p-tolylsulfanyl-isoquinoline-3-carbonyl)-amino]-acetic acid; {[4-Hydroxy-1-(pyridin-2-ylsulfanyl)-isoquinoline-3-carbonyl]-amino}-acetic acid; {[4-Hydroxy-1-(3-methoxy-phenylsulfanyl)-isoquinoline-3-carbonyl]-amino}-acetic acid; {[4-Hydroxy-1-(2-methoxy-phenylsulfanyl)-isoquinoline-3-carbonyl]-amino}-acetic acid; {[4-Hydroxy-1-(naphthalen-2-ylsulfanyl)-isoquinoline-3-carbonyl]-amino}-acetic acid; [(1-Benzenesulfinyl-4-hydroxy-isoquinoline-3-carbonyl)-amino]-acetic acid; [(1-Benzenesulfonyl4-hydroxy-isoquinoline-3-carbonyl)-amino]-acetic acid; {[4-Hydroxy-7-(pyridin-2-ylsulfanyl)-isoquinoline-3-carbonyl]-amino}-acetic acid; {[4-Hydroxy-6-(pyridin-2-ylsulfanyl)-isoquinoline-3-carbonyl]-amino}-acetic acid; [(1-Chloro-4-hydroxy-6,7-diphenoxy-isoquinoline-3-carbonyl)-amino]-acetic acid; [(4-Hydroxy-6,7-diphenoxy-isoquinoline-3-carbonyl)-amino]-acetic acid; ({4-Hydroxy-7-[4-(toluene-4-sulfonylamino)-phenoxy]-isoquinoline-3-carbonyl}-amino)-acetic acid; {[4-Hydroxy-7-(4-nitro-phenoxy)-isoquinoline-3-carbonyl]-amino}-acetic acid; [(4-Mercapto-7-phenoxy-isoquinoline-3-carbonyl)-amino]-acetic acid; [(4-Mercapto-7-trifluoromethyl-isoquinoline-3-carbonyl)-amino]-acetic acid; {[7-(4-Benzenesulfonylamino-phenoxy)-4-hydroxy-isoquinoline-3-carbonyl]-amino-acetic acid; {[4-Hydroxy-7-(4-methanesulfonylamino-phenoxy)-isoquinoline-3-carbonyl]-amino}-acetic acid; [7-(4-Chloro-phenoxy)4-hydroxy-isoquinoline-3-carbonyl]-amino}-acetic acid; {[6-(4-Chloro-phenoxy)-4-hydroxy-isoquinoline-3-carbonyl]-amino}-acetic acid; {[6-(3-Fluoro-5-methoxy-phenoxy)-4-hydroxy-isoquinoline-3-carbonyl]-amino}-acetic acid; {[7-(3-Fluoro-5-methoxy-phenoxy)-4-hydroxy-isoquinoline-3-carbonyl]-amino}-acetic acid; {[7-(3,4-Difluoro-phenoxy)-4-hydroxy-isoquinoline-3-carbonyl]-amino}-acetic acid; {[6-(3,4-Difluoro-phenoxy)-4-hydroxy-isoquinoline-3-carbonyl]-amino}-acetic acid; {[4-Hydroxy-7-(4-trifluoromethoxy-phenoxy)-isoquinoline-3-carbonyl]-amino-acetic acid; {[4-Hydroxy-6-(4-trifluoromethoxy-phenoxy)-isoquinoline-3-carbonyl]-amino-acetic acid; 2-(S)-{[7-(4-Chloro-phenoxy)4-hydroxy-isoquinoline-3-carbonyl]-amino}-propionic acid; 2-(S)-{[6-(4-Chloro-phenoxy)-4-hydroxy-isoquinoline-3-carbonyl]-amino}-propionic acid; 2-{[7-(3,4-Difluoro-phenoxy-4-hydroxy-isoquinoline-3-carbonyl]-amino}-propionic acid; 2-(S)-[(4-Hydroxy-7-phenylsulfanyl-isoquinoline-3-carbonyl)-amino]-propionic acid.; 2-(R)-[(4-Hydroxy-7-phenylsulfanyl-isoquinoline-3-carbonyl)-amino]-propionic acid; 2-(R)-[(4-Hydroxy-7-phenoxy-isoquinoline-3-carbonyl)-amino]-propionic acid; 2-(S)-{[4-Hydroxy-7-(4-methoxy-phenoxy)-isoquinoline-3-carbonyl]-amino}-propionic acid; 2-(S)-[(7-Benzenesulfonyl-4-hydroxy-isoquinoline-3-carbonyl)-amino]-propionic acid; (R)-2-[(4-Hydroxy-1-methoxymethyl-7-phenoxy-isoquinoline-3-carbonyl)-amino]-propionic acid; (S)-2-[(4-Hydroxy-1-methoxymethyl-7-phenoxy-isoquinoline-3-carbonyl)-amino]-propionic acid; (S)-2-[(4-Mercapto-7-phenoxy-isoquinoline-3-carbonyl)-amino]-propionic acid; (S)-2-{[1-(4-Chloro-phenylsulfanyl)4-hydroxy-isoquinoline-3-carbonyl]-amino}-propionic acid; (R)-2-{[1-(4-Chloro-phenylsulfanyl)4-hydroxy-isoquinoline-3-carbonyl]-amino}-propionic acid; [(4-Hydroxy-7-phenylsulfanyl-isoquinoline-3-carbonyl)-amino]-acetic acid; [(4-Hydroxy-6-phenylsulfanyl-isoquinoline-3-carbonyl)-amino]-acetic acid; [(1-Chloro-4-hydroxy-7-phenylsulfanyl-isoquinoline-3-carbonyl)-amino]-acetic acid; [(1-Chloro-4-hydroxy-6-phenylsulfanyl-isoquinoline-3-carbonyl)-amino]-acetic acid; [(1-bromo-4-hydroxy-7-phenylsulfanyl-isoquinoline-3-carbonyl)-amino]-acetic acid; [(1-Bromo-4-hydroxy-6-phenylsulfanyl-isoquinoline-3-carbonyl)-amino]-acetic acid; [(4-Hydroxy-7-phenoxy-isoquinoline-3-carbonyl)-amino]-acetic acid; [(4-Hydroxy-6-phenoxy-isoquinoline-3-carbonyl)-amino]-acetic acid; [(1-Chloro-4-hydroxy-7-phenoxy-isoquinoline-3-carbonyl)-amino]-acetic acid; [(1-Chloro-4-hydroxy-6-phenoxy-isoquinoline-3-carbonyl)-amino]-acetic acid; [(1-Bromo-4-hydroxy-7-phenoxy-isoquinoline-3-carbonyl)-amino]-acetic acid; [(1-Bromo-4-hydroxy-6-phenoxy-isoquinoline-3-carbonyl)-amino]-acetic acid; {[7-(2,6-Dimethyl-phenoxy)-4-hydroxy-isoquinoline-3-carbonyl]-amino}-acetic acid; {[1-Chloro-7-(2,6-dimethyl-phenoxy)-4-hydroxy-isoquinoline-3-carbonyl]-amino}-acetic acid; {[1-bromo-7-(2,6-dimethyl-phenoxy)-4-hydroxy-isoquinoline-3-carbonyl]-amino}-acetic acid; [(1-bromo-7-chloro-4-hydroxy-isoquinoline-3-carbonyl)-amino]-acetic acid; [(1-bromo-6-chloro-4-hydroxy-isoquinoline-3-carbonyl)-amino]-acetic acid; [(1-bromo-4-hydroxy-7-trifluoromethyl-isoquinoline-3-carbonyl)-amino]-acetic acid; [(1-bromo-4-hydroxy-6-trifluoromethyl-isoquinoline-3-carbonyl)-amino]-acetic acid; [(4-Hydroxy-1-phenoxy-isoquinoline-3-carbonyl)-amino]-acetic acid; [(1,7-dibromo-4-hydroxy-isoquinoline-3-carbonyl)-amino]-acetic acid; [(7-bromo-1-chloro-4-hydroxy-isoquinoline-3-carbonyl)-amino]-acetic acid; [(6-bromo-4-hydroxy-isoquinoline-3-carbonyl)-amino]-acetic acid; [(1-bromo-7-fluoro-4-hydroxy-isoquinoline-3-carbonyl)-amino]-acetic acid; [(7-Fluoro-4-hydroxy-isoquinoline-3-carbonyl)-amino]-acetic acid; [(1-Chloro-7-fluoro-4-hydroxy-isoquinoline-3-carbonyl)-amino]-acetic acid; [(1-Chloro-4-hydroxy-benzo[g]isoquinoline-3-carbonyl)-amino]-acetic acid; [(1-bromo-4-hydroxy-isoquinoline-3-carbonyl)-amino]-acetic acid; [(4-Hydroxy-6-phenyl-isoquinoline-3-carbonyl)-amino]-acetic acid; [(4-Hydroxy-7-phenyl-isoquinoline-3-carbonyl)-amino]-acetic acid; [(1-Chloro-4-hydroxy-6-phenyl-isoquinoline-3-carbonyl)-amino]-acetic acid; [(1-Chloro-4-hydroxy-7-phenyl-isoquinoline-3-carbonyl)- amino]-acetic acid; [(1-Bromo-4-hydroxy-6-phenyl-isoquinoline-3-carbonyl)-amino]-acetic acid; [(1-bromo-4-hydroxy-7-phenyl-isoquinoline-3-carbonyl)-amino]-acetic acid; [(4-Hydroxy-5-phenyl-isoquinoline-3-carbonyl)-amino]-acetic acid; [(4-Hydroxy-8-phenyl-isoquinoline-3-carbonyl)-amino]-acetic acid; [(1-Chloro-4-hydroxy-5-phenyl-isoquinoline-3-carbonyl)-amino]-acetic acid; [(1-Chloro-4-hydroxy-8-phenyl-isoquinoline-3-carbonyl)-amino]-acetic acid; [(1-bromo-4-hydroxy-5-phenyl-isoquinoline-3-carbonyl)-amino]-acetic acid; [(1-bromo-4-hydroxy-8-phenyl-isoquinoline-3-carbonyl)-amino]-acetic acid; [(1-Ethylsulfanyl-4-hydroxy-isoquinoline-3-carbonyl)-amino]-acetic acid; {[4-Hydroxy-1-(4-methoxy-phenylsulfanyl)-isoquinoline-3-carbonyl]-amino}-acetic acid; [(1-Chloro-4-hydroxy-7-iodo-isoquinoline-3-carbonyl)-amino]-acetic acid; [(1-Chloro-4-hydroxy-6-iodo-isoquinoline-3-carbonyl)-amino]-acetic acid; [(4-Hydroxy-7-iodo-isoquinoline-3-carbonyl)-amino]-acetic acid; [(1-bromo-4-hydroxy-7-methyl-isoquinoline-3-carbonyl)-amino]-acetic acid; [(1-bromo-7-butoxy-4-hydroxy-isoquinoline-3-carbonyl)-amino]-acetic acid; [(1-bromo-6-butoxy-4-hydroxy-isoquinoline-3-carbonyl)-amino]-acetic acid; [(6-Benzyloxy-1-Chloro-4-hydroxy-isoquinoline-3-carbonyl)-methyl-amino]-acetic acid; [(1-Chloro-4-hydroxy-isoquinoline-3-carbonyl)-methyl-amino]-acetic acid; [(1-Chloro-4-hydroxy-6-isopropoxy-isoquinoline-3-carbonyl)-methyl-amino]-acetic acid; [(1-Chloro-4-hydroxy-7-isopropoxy-isoquinoline-3-carbonyl)-methyl-amino]-acetic acid; [Carboxymethyl-(1-Chloro-4-hydroxy-6isopropoxy-isoquinoline-3-carbonyl)-amino]-acetic acid; [Carboxymethyl-(1-Chloro-4-hydroxy-6-isopropoxy-isoquinoline-3-carbonyl)-amino]-acetic acid; 1-Chloro-4-hydroxy-isoquinoline-3-carboxylic acid (2-amino-ethyl)-amide (trifluoro-acetic acid salt); 1-Chloro-4-hydroxy-isoquinoline-3-carboxylic acid (2-methoxy-ethyl)-amide; 1-Chloro-4-hydroxy-isoquinoline-3-carboxylic acid (2-hydroxy-ethyl)-amide; 1-Chloro-4-hydroxy-isoquinoline-3-carboxylic acid (2-dimethylamino-ethyl)-amide; 1-Chloro-4-hydroxy-isoquinoline-3-carboxylic acid (2-acetylamino-ethyl)-amide; 1-Chloro-4-hydroxy-6-isopropoxy-isoquinoline-3-carboxylic acid (2-hydroxy-ethyl)-amide; 1-Chloro-4-hydroxy-6-isopropoxy-isoquinoline-3-carboxylic acid (2-methoxy-ethyl)-amide; 1-Chloro-4-hydroxy-6-isopropoxy-isoquinoline-3-carboxylic acid (2-amino-ethyl)-amide (trifluoro-acetic acid salt); 1-Chloro-4-hydroxy-6-isopropoxy-isoquinoline-3-carboxylic acid (2-dimethylamino-ethyl)-amide; 1-Chloro-4-hydroxy-7-isopropoxy-isoquinoline-3-carboxylic acid (2-amino-ethyl)-amide (trifluoro-acetic acid salt); 1-Chloro-4-hydroxy-7-isopropoxy-isoquinoline-3-carboxylic acid (2-methoxy-ethyl)-amide; 1-Chloro-4-hydroxy-7-isopropoxy-isoquinoline-3-carboxylic acid (2-dimethylamino-ethyl)-amide; 1-Chloro-4-hydroxy-7-isopropoxy-isoquinoline-3-carboxylic acid (2-hydroxy-ethyl)-amide; (S)-2-[(6-Benzyloxy-1-Chloro-4-hydroxy-isoquinoline-3-carbonyl)-amino]-propionic acid; (R)-2-[(1-Chloro-4-hydroxy-isoquinoline-3-carbonyl)-amino]-3-hydroxy-propionic acid; (S)-2-[(1-Chloro-4-hydroxy-isoquinoline-3-carbonyl)-amino]-3-hydroxy-propionic acid; (R)-2-[(1-Chloro-4-hydroxy-6-isopropoxy-isoquinoline-3-carbonyl)-amino]-3-hydroxy-propionic acid; (S)-2-[(1-Chloro-4-hydroxy-6-isopropoxy-isoquinoline-3-carbonyl)-amino]-3-hydroxy-propionic acid; (R)-2-[(1-Chloro-4-hydroxy-7-isopropoxy-isoquinoline-3-carbonyl)-amino]-3-hydroxy-propionic acid; (S)-2-[(1-Chloro-4-hydroxy-7-isopropoxy-isoquinoline-3-carbonyl)-amino]-3-hydroxy-propionic acid; 2-[(1-Chloro-4-hydroxy-isoquinoline-3-carbonyl)-amino]-2-methyl-propionic acid; 2-[(1-Chloro-4-hydroxy-6-isopropoxy-isoquinoline-3-carbonyl)-amino]-2-methyl-propionic acid; (R)-2-[(1-Chloro-4-hydroxy-isoquinoline-3-carbonyl)-amino]-3-(1H-imidazol-4-yl)-propionic acid (trifluoro-acetic acid salt); (S)-2-[(1-Chloro-4-hydroxy-isoquinoline-3-carbonyl)-amino]-3-(1H-imidazol4-yl)-propionic acid (trifluoro-acetic acid salt); (R)-2-[(1-Chloro-4-hydroxy-isoquinoline-3-carbonyl)-amino]-3-methyl-butyric acid; (S)-2-[(1-Chloro-4-hydroxy-isoquinoline-3-carbonyl)-amino]-3-methyl-butyric acid; (R)-2-[(1-Chloro-4-hydroxy-6-isopropoxy-isoquinoline-3-carbonyl)-amino]-3-methyl-butyric acid; (S)-2-[(1-Chloro-4-hydroxy-6-isopropoxy-isoquinoline-3-carbonyl)-amino]-3-methyl-butyric acid; (R)-2-[(1-Chloro-4-hydroxy-7-isopropoxy-isoquinoline-3-carbonyl)-amino]-3-methyl-butyric acid; (S)-2-[(1-Chloro-4-hydroxy-7-isopropoxy-isoquinoline-3-carbonyl)-amino]-3-methyl-butyric acid; (S)-2-[(6-Benzyloxy-1-Chloro-4-hydroxy-isoquinoline-3-carbonyl)-amino]-3-methyl-butyric acid; (R)-2-[(1-Chloro-4-hydroxy-isoquinoline-3-carbonyl)-amino]-3-phenyl-propionic acid; (S)-2-[(1-Chloro-4-hydroxy-isoquinoline-3-carbonyl)-amino]-3-phenyl-propionic acid; (R)-2-[(1-Chloro-4-hydroxy-6-isopropoxy-isoquinoline-3-carbonyl)-amino]-3-phenyl-propionic acid; (S)-2-[(1-Chloro-4-hydroxy-6-isopropoxy-isoquinoline-3-carbonyl)-amino]-3-phenyl-propionic acid; (R)-2-[(1-Chloro-4-hydroxy-7-isopropoxy-isoquinoline-3-carbonyl)-amino]-3-phenyl-propionic acid; (S)-2-[(1-Chloro-4-hydroxy-7-isopropoxy-isoquinoline-3-carbonyl)-amino]-3-phenyl-propionic acid; (R)-2-[(1-Chloro-4-hydroxy-isoquinoline-3-carbonyl)-amino]-3-(4-hydroxy-phenyl)-propionic acid; (S)-2-[(1-Chloro-4-hydroxy-isoquinoline-3-carbonyl)-amino]-3-(4-hydroxy-phenyl)-propionic acid; (R)-2-[(1-Chloro-4-hydroxy-6-isopropoxy-isoquinoline-3-carbonyl)-amino]-3-(4-hydroxy-phenyl)-propionic acid; (S)-2-[(1-Chloro-4-hydroxy-6-isopropoxy-isoquinoline-3-carbonyl)-amino]-3-(4-hydroxy-phenyl)-propionic acid; (R)-2-[(1-Chloro-4-hydroxy-7-isopropoxy-isoquinoline-3-carbonyl)-amino]-3-(4-hydroxy-phenyl)-propionic acid; (S)-2-[(1-Chloro-4-hydroxy-7-isopropoxy-isoquinoline-3-carbonyl)-amino]-3-(4-hydroxy-phenyl)-propionic acid; (R)-2-[(1-Chloro-4-hydroxy-6-isopropoxy-isoquinoline-3-carbonyl)-amino]-pentanoic acid; (S)-2-[(1-Chloro-4-hydroxy-6-isopropoxy-isoquinoline-3-carbonyl)-amino]-pentanoic acid; (R)-1-(1-Chloro-4-hydroxy-isoquinoline-3-carbonyl)-pyrrolidine-2-carboxylic acid; (S)-1-(1-Chloro-4-hydroxy-isoquinoline-3-carbonyl)-pyrrolidine-2-carboxylic acid; (R)-1-(1-Chloro-4-hydroxy-6-isopropoxy-isoquinoline-3-carbonyl)-pyrrolidine-2-carboxylic acid; (S)-1-(1-Chloro-4-hydroxy-6-isopropoxy-isoquinoline-3-carbonyl)-pyrrolidine-2-carboxylic acid; (R)-6-Amino-2-[(1-chloro-4-hydroxy-isoquinoline-3-carbonyl)-amino]-hexanoic acid (trifluoro-acetic acid salt); (S)-6-Amino-2-[(1-chloro-4-hydroxy-isoquinoline-3-carbonyl)-amino]-hexanoic acid (trifluoro-acetic acid salt); (R)-6-Amino-2-[(1-Chloro-4-hydroxy-6-isopropoxy-isoquinoline-3-carbonyl)-amino]-hexanoic acid; trifluoroacetic acid salt; (S)-6-Amino-2-[(1-Chloro-4-hydroxy-6-isopropoxy-isoquinoline-3-carbonyl)-amino]-hexanoic acid (trifluoro-acetic acid salt); (R)-6-Amino-2-[(1-Chloro-4-hydroxy-7-isopropoxy-isoquinoline-3-carbonyl)-amino]-hexanoic acid; trifluoroacetic acid salt; (S)-6-Amino-2-[(1-chloro-4-hydroxy-7-isopropoxy-isoquinoline-3-carbonyl)-amino]-hexanoic acid (trifluoro-acetic acid salt); (R)-2-[(1-Chloro-4-hydroxy-isoquinoline-3-carbonyl)-amino]-succinic acid; (S)-2-[(1-Chloro-4-hydroxy-isoquinoline-3-carbonyl)-amino]-succinic acid; (R)-2-[(1-Chloro-4-hydroxy-6-isopropoxy-isoquinoline-3-carbonyl)-amino]-succinic acid; (S)-2-[(1-Chloro-4-hydroxy-6-isopropoxy-isoquinoline-3-carbonyl)-amino]-succinic acid; (R)-2-[(1-Chloro-4-hydroxy-7-isopropoxy-isoquinoline-3-carbonyl)-amino]-succinic acid; 1-[(1-Chloro-4-hydroxy-isoquinoline-3-carbonyl)-amino]-cyclopropanecarboxylic acid; 1-[(1-Chloro-4-hydroxy-6-isopropoxy-isoquinoline-3-carbonyl)-amino]-cyclopropanecarboxylic acid; Dideutero-[(1-Chloro-4-hydroxy-isoquinoline-3-carbonyl)-amino]-acetic acid; (R)-2-[(6-Benzyloxy-1-Chloro-4-hydroxy-isoquinoline-3-carbonyl)-amino]-propionic acid; (S)-2-[(7-Benzyloxy-1-Chloro-4-hydroxy-isoquinoline-3-carbonyl)-amino]-propionic acid; (R)-2-[(7-Benzyloxy-1-Chloro-4-hydroxy-isoquinoline-3-carbonyl)-amino]-propionic acid; (S)-2-[(1-Chloro-4-hydroxy-isoquinoline-3-carbonyl)-amino]-propionic acid; (R)-2-[(1-Chloro-4-hydroxy-isoquinoline-3-carbonyl)-amino]-propionic acid; (S)-2-[(6-Isopropoxy-1-Chloro-4-hydroxy-isoquinoline-3-carbonyl)-amino]-propionic acid; (R)-2-[6-Isopropoxy-1-Chloro-4-hydroxy-isoquinoline-3-carbonyl)-amino]-propionic acid; (S)-2-[(7-Isopropoxy-1-Chloro-4-hydroxy-isoquinoline-3-carbonyl)-amino-propionic acid; (R)-2-[(7-Isopropoxy-1-Chloro-4-hydroxy-isoquinoline-3-carbonyl)-amino]propionic acid; 1-Chloro-4-hydroxy-6-isopropoxy-isoquinoline-3-carboxylic acid (2-hydroxy-1-hydroxymethyl-ethyl)-amide; 1-Chloro-4-hydroxy-7-isopropoxy-isoquinoline-3-carboxylic acid (2-hydroxy-1-hydroxymethyl-ethyl)-amide; 1-Chloro-4-hydroxy-isoquinoline-3-carboxylic acid (2-hydroxy-1-hydroxymethyl-ethyl)-amide; {[7-(3,5-Difluoro-phenoxy)-4-hydroxy-isoquinoline-3-carbonyl]-amino}-acetic acid; {[6-(3,5-Difluoro-phenoxy)-4-hydroxy-isoquinoline-3-carbonyl]-amino}-acetic acid; ({7-[4-(4-Fluoro-phenoxy)-phenoxy]4-hydroxy-isoquinoline-3-carbonyl}-amino)-acetic acid; ({6-[4-(4-Fluoro-phenoxy)-phenoxy]-4-hydroxy-isoquinoline-3-carbonyl}-amino)-acetic acid; {[7-(3-Chloro-4-fluoro-phenoxy)-4-hydroxy-isoquinoline-3-carbonyl]-amino}-acetic acid; {[6-(3-Chloro-4-fluoro-phenoxy)-4-hydroxy-isoquinoline-3-carbonyl]-amino}-acetic acid; (S)-2-{[7-(3-fluoro-5-methoxy-phenoxy)-4-hydroxy-isoquinoline-3-carbonyl]-amino}-propionic acid; 2-(S)-[(7-Cyclohexyloxy-4-hydroxy-isoquinoline-3-carbonyl)-amino]-propionic acid; 2-(S)-{[7-(4-Fluoro-phenoxy)-4-hydroxy-1-methyl-isoquinoline-3-carbonyl]-amino]-propionic acid; 2-(S}-{[7-(4-Fluoro-phenoxy)-4-hydroxy-isoquinoline-3-carbonyl]-amino}-acid; 2-(S)-[(4-Hydroxy-1-methyl-7-phenoxy-isoquinoline-3-carbonyl)-amino]-propionic acid; 2-(S)-[(4-Hydroxy-1-methyl-7-phenylsulfanyl-isoquinoline-3-carbonyl)-amino]-propionic acid; 2-(S)-{[4-Hydroxy-7-(4-trifluoromethyl-phenoxy)-isoquinoline-3-carbonyl]-amino}-propionic acid; {[7-(4-Chloro-phenoxy)-4-hydroxy-1-methyl-isoquinoline-3-carbonyl]-amino}-acetic acid; {[6-(4-Chloro-phenoxy)-1-methyl-isoquinoline-3-carbonyl]-amino}-acetic acid; {[7-(3,5-Difluoro-phenoxy)-4-hydroxy-1-methyl-isoquinoline-3-carbonyl]-amino}-acetic acid; {[4-Hydroxy-7-(4-methoxy-phenoxy)-1-methyl-isoquinoline-3-carbonyl]-amino}-acetic acid; {[4-Hydroxy-6-(4-methoxy-phenoxy)-1-methyl-isoquinoline-3-carbonyl]-amino}-acetic acid; [(6-Cyclohexyloxy-4-hydroxy-isoquinoline-3-carbonyl)-amino]-acetic acid; [(7-Cyclohexyloxy-4-hydroxy-isoquinoline-3-carbonyl)-amino]-acetic acid; [(7-Cyclohexyloxy-4-hydroxy-1-methyl-isoquinoline-3-carbonyl)-amino]-acetic acid; [(7-Cyclohexylsulfanyl-4-hydroxy-isoquinoline-3-carbonyl)-amino]-acetic acid; [(7-Cyclohexanesulfonyl-4-hydroxy-isoquinoline-3-carbonyl)-amino]-acetic acid; [(4-Hydroxy-1-isobutyl-isoquinoline-3-carbonyl)-amino]-acetic acid; [(4-Hydroxy-1-pyridin-2-yl-isoquinoline-3-carbonyl)-amino]-acetic acid; [(1-Ethyl-4-hydroxy-7-phenoxy-isoquinoline-3-carbonyl)-amino]-acetic acid; [(1-Dimethylaminomethyl-4-hydroxy-7-phenylsulfanyl-isoquinoline-3-carbonyl)-amino]-acetic acid; [(4-Hydroxy-1-methyl-7-phenylsulfanyl-isoquinoline-3-carbonyl)-amino]-acetic acid; and {[4-Hydroxy-1-methyl-7-(4-trifluoromethyl-phenoxy)-isoquinoline-3-carbonyl]-amino}-acetic acid.

In other embodiments, the agent is selected from the group consisting of: [(1-Chloro-4-hydroxy-isoquinoline-3-carbonyl)-amino]-acetic acid, [(4-Hydroxy-1-methyl-7-phenoxy-isoquinoline-3-carbonyl)-amino]-acetic acid, {[4-Hydroxy-7-(4-methoxy-phenoxy)-isoquinoline-3-carbonyl]-amino}-acetic acid, [(4-Hydroxy-8-phenoxy-isoquinoline-3-carbonyl)-amino]-acetic acid, [(4-Hydroxy-1-methyl-8-phenoxy-isoquinoline-3-carbonyl)-amino]-acetic acid, [(7-Chloro-4-hydroxy-1-methyl-isoquinoline-3-carbonyl)-amino]-acetic acid, {[8-(4-Fluoro-phenoxy)-4-hydroxy-1-methyl-isoquinoline-3-carbonyl]-amino}acetic acid, and [(4-cyano-7-hydroxy-thieno[3,2-c]pyridine-6-carbonyl)-amino]-acetic acid.

While, as discussed herein, a preferred agent of the present invention is a small molecule compound, it is contemplated herein that inhibiting HIF hydroxylase activity can be accomplished by any of the methods available to and known by those of skill in the art, and can involve use of any agent that interacts with, binds to, or modifies HIFα or factors that interact with HIFα, including, e.g., enzymes for which HIFα is a substrate. In certain aspects, the present invention contemplates providing a constitutively stable HIFα variant, e.g., stable HIF muteins, etc, or a polynucleotide encoding such a variant. In further aspects, HIFαis HIF1α, HIF2═, or HIF3α. In a preferred aspect, inhibiting HIF hydroxylase activity comprises administering to the subject an effective amount of an agent that inhibits HIF prolyl hydroxylase activity.

Pharmaceutical compositions or medicaments effective for treating anemia, increasing hemoglobin levels, and increasing hematocrit are also provided herein, wherein the anemia treatment, the increased hemoglobin levels, or the increased hematocrit are associated with a lower risk of thrombosis or hypertension compared to that observed with recombinant human EPO therapy. In various embodiments, the compositions comprise an effective amount of an agent that inhibits HIF hydroxylase activity and a carrier. A pharmaceutical composition effective for treating anemia, increasing hemoglobin levels, and increasing hematocrit, the composition comprising an effective amount of an agent that inhibits HIF hydroxylase activity is specifically contemplated.

In various embodiments, the agent is administered orally, systemically, by injection, and intravenously. In certain embodiments, the subject is a mammalian subject, including, e.g., a cat, a dog, etc. In preferred embodiments, the subject is a human subject.

In one embodiment, the subject is resistant or hyporesponsive to erythropoiesis stimulating protein treatment, including recombinant human EPO treatment. In another embodiment, the subject has previously been treated with recombinant human EPO therapy.

In one embodiment, the subject has one or more risk factors for developing thrombosis. In another embodiment, the subject has one of more risk factors for developing hypertension.

It is contemplated, in various embodiments, that the methods and agents of the present invention are used in conjunction with recombinant human EPO therapy. In certain aspects, the agent administered in simultaneous, separate, or sequential administration with recombinant human EPO. It is further contemplated that, in various embodiments, that the methods and agents of the present invention are used in conjunction with iron supplement therapy, with an agent for reducing thrombosis, or with an agent for reducing hypertension. I

n certain embodiments, the agent is administered at a dose of 3 mg/kg, 6 mg/kg, 10 mg/kg, 15 mg/kg, 20 mg/kg or 30 mg/kg. In other embodiments, the agent is administered two or three times weekly.

These and other embodiments of the subject invention will readily occur to those of skill in the art in light of the disclosure herein, and all such embodiments are specifically contemplated.

### BRIEF DESCRIPTION OF THE DRAWINGS

FIG. 1 sets forth data showing methods and compounds of the present invention increased circulating EPO levels in animals.

FIG. 2 sets forth data showing methods and compounds of the present invention increased circulating EPO levels in animals.

FIG. 3 sets forth data showing methods and compounds of the present invention increased circulating EPO levels in human subjects.

FIGS. 4A and 4B set forth data showing methods and compounds of the present invention increased circulating EPO levels in bilateral nephrectomized animals.

FIGS. 5A and 5B set forth data showing methods and compounds of the present invention increased hemoglobin levels in human subject with chronic kidney disease.

FIGS. 6A and 6B set forth data showing methods and compounds of the present invention increased hemoglobin levels in human subject with chronic kidney disease.

### DESCRIPTION OF THE INVENTION

Compounds

These methods of the present invention are effected by administering a compound that stabilizes HIF; preferably, a compound that inhibits HIF prolyl hydroxylase activity.

Exemplary compounds are disclosed in, e.g., WO 2004/108121 and WO 2004/108681, incorporated herein by reference in their entireties.

Preferably, the compounds used in the present invention inhibit HIF hydroxylase activity, as disclosed in WO 2004/108121 and WO 2004/108681. In various embodiments, the activity is due to a HIF prolyl hydroxylase, such as, for example, EGLN1, EGLN2, or EGLN3, etc. In other embodiments, the activity is due to a HIF asparaginyl hydroxylase, such as, for example, including, but not limited to, FIH. A preferred compound of the invention is a compound that inhibits HIF prolyl hydroxylase activity. The inhibition can be direct or indirect, can be competitive or non-competitive, etc.

Preferred compounds for use in the present invention include [(1-Chloro-4-hydroxy-isoquinoline-3-carbonyl)-amino]-acetic acid (Compound A), [(4-Hydroxy-1-methyl-7-phenoxy-isoquinoline-3-carbonyl)-amino]-acetic acid (Compound B), {[4-Hydroxy-7-(4-methoxy-phenoxy)-isoquinoline-3-carbonyl]-amino}-acetic acid (Compound C). [(4-Hydroxy-8-phenoxy-isoquinoline-3-carbonyl)-amino]-acetic acid (Compound D), [(4-Hydroxy-1-methyl-8-phenoxy-isoquinoline-3-carbonyl)-amino]-acetic acid (Compound E), [(7-Chloro-4-hydroxy-1-methyl-isoquinoline-3-carbonyl)-amino]-acetic acid (Compound F), {[8-(4-Fluoro-phenoxy)-4-hydroxy-1-methyl-isoquinoline-3-carbonyl]-amino}-acetic acid (Compound G), and [(4-cyano-7-hydroxy-thieno[3,2-c]pyridine-6-carbonyl)-amino]-acetic acid (Compound H ). Particularly preferred is [(1-Chloro-4-hydroxy-isoquinoline-3-carbonyl)-amino]-acetic acid (Compound A).

In one aspect, a compound of the invention is any compound that inhibits or otherwise modulates the activity of a 2-oxoglutarate dioxygenase enzyme. 2-oxoglutarate dioxygenase enzymes include, but are not limited to, hydroxylase enzymes. Hydroxylase enzymes hydroxylate target substrate residues and include, for example, prolyl, lysyl, asparaginyl (asparagyl, aspartyl) hydroxylases, etc. Hydroxylases are sometimes described by target substrate, e.g., HIF hydroxylases, procollagen hydroxylases, etc., and/or by targeted residues within the substrate, e.g., prolyl hydroxylases, lysyl hydroxylases, etc., or by both, e.g., HIF prolyl hydroxylases, procollagen prolyl hydroxylases, etc. Representative 2-oxoglutarate dioxygenase enzymes include, but are not limited to, HIF hydroxylases, including HIF prolyl hydroxylases, e.g., EGLN1, EGLN2, and EGLN3, HIF asparaginyl hydroxylases, e.g., factor inhibiting HIF (FIH), etc.; procollagen hydroxylases, e.g., procollagen lysyl hydroxylases, procollagen prolyl hydroxylases, e.g., procollagen prolyl 3-hydroxylase, procollagen prolyl 4-hydroxylase a(I) and a(II), etc.; thymine 7-hydroxylase; aspartyl (asparaginyl) β-hydroxylase; _{ε}-N-trimethyllysine hydroxylase; γ-butyrobetaine hydroxylase, etc. Although enzymatic activity can include any activity associated with any 2-oxoglutarate dioxygenase, the hydroxylation of amino acid residues within a substrate is specifically contemplated. Although hydroxylation of proline and/or asparagine residues within a substrate is specifically included, hydroxylation of other amino acids is also contemplated.

In one aspect, a compound of the invention that shows inhibitory activity toward one or more 2-oxoglutarate dioxygenase enzyme may also show inhibitory activity toward one or more additional 2-oxoglutarate dioxygenase enzymes, e.g., a compound that inhibits the activity of a HIF hydroxylase may additionally inhibit the activity of a collagen prolyl hydroxylase, a compound that inhibits the activity of a HIF prolyl hydroxylase may additionally inhibit the activity of a HIF asparaginyl hydroxylase, etc.

As HIFα is modified by proline hydroxylation, a reaction requiring oxygen and Fe²⁺, the present invention contemplates in one aspect that the enzyme responsible for HIFα hydroxylation is a member of the 2-oxoglutarate dioxygenase family. Such enzymes include, but are not limited to, procollagen lysyl hydroxylase, procollagen prolyl 3-hydroxylase, procollagen prolyl 4-hydroxylase α(I) and α(II), thymine 7-hydroxylase, aspartyl (asparaginyl) β-hydroxylase, _{ε}-N-trimethyllysine hydroxylase, and γ-butyrobetaine hydroxylase, etc. These enzymes require oxygen, Fe²⁺, 2-oxoglutarate, and ascorbic acid for their hydroxylase activity. (See, e.g., Majamaa et al. (1985) Biochem J 229:127-133; Myllyharju and Kivirikko (1997) EMBO J 16:1173-1180; Thornburg et al. (1993) 32:14023-14033; and Jia et al. (1994) Proc Natl Acad Sci USA 91:7227-7231.)

In one aspect, a compound of the invention is a compound that stabilizes HIFα Preferably, the compound stabilizes HIFα through inhibition of HIF hydroxylase activity. It is thus specifically contemplated that a compound of the invention may be selected from previously identified modulators of hydroxylase activity. For example, small molecule inhibitors of prolyl 4-hydroxylase have been identified. (See, e.g., Majamaa et al. (1984) Eur J Biochem 138:239-245; Majamaa et al.(1985) Biochem J 229:127-133; Kivirikko and Myllyharju (1998) Matrix Biol 16:357-368; Bickel et al. (1998) Hepatology 28:404411; Friedman et al. (2000) Proc Natl Acad Sci USA 97:47364741; and Franklin et al. (2001) Biochem J 353:333-338; all incorporated by reference herein in their entirety.) The present invention contemplates the use of these compounds in the methods provided herein.

In some aspects, compounds of the present invention include, for example, structural mimetics of 2-oxoglutarate. Such compounds may inhibit the target 2-oxoglutarate dioxygenase enzyme family member competitively with respect to 2-oxoglutarate and noncompetitively with respect to iron. (Majamaa et al. (1984) Fur J Biochem. 138:239-245; and Majamaa et al. Biochem J 229:127-133.)

In certain embodiments, compounds used in the methods of the invention are selected from a compound of the formula (I) 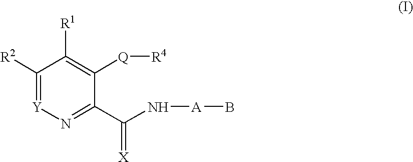
wherein
A is 1,2-arylidene, 1,3-arylidene, 1,4-arylidene; or (C₁-C₄)-alkylene, optionally substituted by one or two halogen, cyano, nitro, trifluoromethyl, (C1-C₆)-alkyl, (C₁-C₆)-hydroxyalkyl, (C₁-C₆)-alkoxy, —O—[CH₂]₂—C_{f}H_{(2f+1-g)}Hal_{g}, (C₁-C₆)-fluoroalkoxy, (C₁-C₈)-fluoroalkenyloxy, (C₁-C₈) -fluoroalkynyloxy, —OCF₂Cl, —O—CF₂—CHFCl; (C₁-C₆)-alkylmercapto, (C₁-C₆)-alkylsulfinyl, (C₁-C₆)-alkylsulfonyl, (C₁-C₆)-alkylcarbonyl, (C₁-C₆)-alkoxycarbonyl, carbamoyl, N—(C₁-C₄)-alkylcarbamoyl, N,N-di-(C₁-C₄)-alkylcarbamoyl, (C₁-C₆)-alkylcarbonyloxy, (C₃-C₈)-cycloalkyl, phenyl, benzyl, phenoxy, benzyloxy, anilino, N-methylanilino, phenylmercapto, phenylsulfonyl, phenylsulfinyl, sulfamoyl, N—(C₁-C₄)-alkylsulfamoyl, N,N-di-(C₁-C₄)-alkylsulfamoyl; or by a substituted (C₆-C₁₂)-aryloxy, (C₇-C₁₁)-aralkyloxy, (C₆-C₁₂)-aralkyl, (C₇-C₁₁)-aralkyl radical, which carries in the aryl moiety one to five identical or different substituents selected from halogen, cyano, nitro, trifluoromethyl, (C1-C₆)-alkyl, (C₁-C₆)-alkoxy, —O—[CH₂]ₓ—C_{f}H_{(2f+1-g)}Hal_{g}, —OCF₂Cl, —O—CF₂—CHFCl, (C₁-C₆)-alkylmercapto, (C₁-C₆)-alkylsulfinyl, (C₁-C₆)-alkylsulfonyl, (C₁-C₆)-alkylcarbonyl, (C₁-C₁)-alkoxycarbonyl, carbamoyl, N—(C₁-C₄)-alkylcarbamoyl, N,N-di-(C₁-C₄)-alkylcarbamoyl, (C₁-C₆)-alkylcarbonyloxy, (C₃-C₈)-cycloalkyl, sulfamoyl, N—(C₁-C₄)-alkylsulfamoyl, N,N-di-(C₁-C₄)-alkylsulfamoyl; or wherein A is —CR⁵R⁶ and R⁵ and R⁶ are each independently selected from hydrogen, (C₁-C₆)-alkyl, (C₃-C₇)-cycloalkyl, aryl, or a substituent of the α-carbon atom of an α-amino acid, wherein the amino acid is a natural L-amino acid or its D-isomer;
B is —CO₂H, —NH₂, —NHSO₂CF₃, tetrazolyl, imidazolyl, 3-hydroxyisoxazolyl, —CONHCOR′″, —CONHSOR′″, CONHSO₂R′″, where R′″ is aryl, heteroaryl, (C₃-C₇)-cycloalkyl, or (C1-C₄)-alkyl, optionally monosubstituted by (C₆-C₁₂)-aryl, heteroaryl, OH, SH, (C₁-C₄)-alkyl, (C₁-C₄)-alkoxy, (C₁-C₄)-thioalkyl, (C₁-C₄)-sulfinyl, (C₁-C₄)-sulfonyl, CF₃, Cl, Br, F, I, NO2,—COOH, (C₂-C₅)-alkoxycarbonyl, NH₂, mono-(C₁-C₄-alkyl)-amino, di-(C₁-C₄-alkyl)-amino, or (C₁-C₄)-perfluoroalkyl; or wherein B is a CO₂-G carboxyl radical, where G is a radical of an alcohol G-OH in which G is selected from (C₁-C₂₀)-alkyl radical, (C₃-C₈) cycloalkyl radical, (C₂-C₂₀)-alkenyl radical, (C₃-C₈)-cycloalkenyl radical, retinyl radical, (C₂-C₂₀)-alkynyl radical, (C₄-C₂₀)-alkenynyl radical, where the alkenyl, cycloalkenyl, alkynyl, and alkenynyl radicals contain one or more multiple bonds; (C₆-C₁₆)-carbocyclic aryl radical, (C₇-C₁₆)-carbocyclic aralkyl radical, heteroaryl radical, or heteroaralkyl radical, wherein a heteroaryl radical or heteroaryl moiety of a heteroaralkyl radical contains 5 or 6 ring atoms; and wherein radicals defined for G are substituted by one or more hydroxyl, halogen, cyano, trifluoromethyl, nitro, carboxyl, (C₁-C₁₂)-alkyl, (C₃-C₈)-cycloalkyl, (C₃-C₈)-cycloalkenyl, (C₆-C₁₂)-aryl, (C₇-C₁₆)-aralkyl, (C₂-C₁₂)-alkenyl, (C₂-C₁₂)-alkynyl, (C₁-C₁₂)-alkoxy, (C₁-C₁₂)-alkoxy-(C₁-C₁₂)-alkyl, (C₁-C₁₂)-alkoxy-(C₁-C₁₂)-alkoxy, (C₆-C₁₂)-aryloxy, (C₇-C₁₆)-aralkyloxy, (C₁-C₈)-hydroxyalkyl, —O—[CH₂]ₓ—C_{f}H_{(2f+1-g)}—F_{g}, —OCF₂Cl, —OCF₂—CHFCl, (C₁-C₁₂)-alkylcarbonyl, (C₃-C₈)-cycloalkylcarbonyl, (C₆-C₁₂)-arylcarbonyl, (C₇-C₁₆)-aralkylcarbonyl, cinnamoyl, (C₂-C₁₂)-alkenylcarbonyl, (C₂-C₁₂)-alkynylcarbonyl, (C₁-C₁₂)-alkoxycarbonyl, (C₁-C₁₂)-alkoxy-(C₁-C₁₂)-alkoxycarbonyl, (C₆-C₁₂)-aryloxycarbonyl, (C₇-C₁₆)-aralkyloxycarbonyl, (C₃-C₈)-cycloalkoxycarbonyl, (C₂-C₁₂)-alkenyloxycarbonyl, (C₂-C₁₂)-alkynyloxycarbonyl, acyloxy, (C₁-C₁₂)-alkoxycarbonyloxy, (C₁-C₁₂)-alkoxy-(C₁-C₁₂)-alkoxycarbonyloxy, (C₆-C₁₂)-aryloxycarbonyloxy, (C₇-C₁₆) aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N—(C₁-C₁₂)-alkylcarbamoyl, N,N-di(C₁-C₁₂)-alkylcarbamoyl, N—(C₃-C₈)-cycloalkyl-carbamoyl, N—(C₆-C₁₆)-arylcarbamoyl, N—(C₇-C₁₆)-aralkylcarbamoyl, N—(C₁-C₁₀)-alkyl-N—(C₆-C₁₆)-arylcarbamoyl, N—(C₁-C₁₀)-alkyl-N—(C₇-C₁₆)-aralkylcarbamoyl, N—((C₁-C₁₀)-alkoxy-(C₁-C₁₀)-alkyl)-carbamoyl, N—((C₆-C₁₂)-aryloxy-(C₁-C₁₀)alkyl)-carbamoyl, N—((C₇-C₁₆)-aralkyloxy-(C₁-C₁₀)-alkyl)-carbamoyl, N—(C₁-C₁₀)-alkyl-N—((C₁-C₁₀)-alkoxy-(C₁-C₁₀)-alkyl)-carbamoyl, N—(C₁-C₁₀)-alkyl-N—((C₆-C₁₆)-aryloxy-(C₁-C₁₀)-alkyl)-carbamoyl, N—(C₁-C₁₀)-alkyl-N—((C₇-C₁₆)-aralkyloxy-(C₁-C₁₀)-alkyl)-carbamoyl, carbamoyloxy, N—(C₁-C₁₂)-alkylcarbamoyloxy, N,N-di-(C₁-C₁₂)-alkylcarbamoyloxy, N—(C₃-C₈)-cycloalkylcarbamoyloxy, N—(C₆-C₁₂)-arylcarbamoyloxy, N—(C₇-C₁₆)-aralkylcarbamoyloxy, N—(C₁-C₁₀)-alkyl-N—(C₆-C₁₂)-arylcarbamoyloxy, N—(C₁-C₁₀)-alkyl-N—(C₇-C₁₆)-aralkylcarbamoyloxy, N—((C₁-C₁₀)-alkyl)-carbamoyloxy, N—((C₆-C₁₂)-aryloxy-(C₁-C₁₀)-alkyl)-carbamoyloxy, N—((C₇-C₁₆)-aralkyloxy-(C₁-C₁₀)-alkyl)-carbamoyloxy, N—(C₁-C₁₀)-alkyl-N—((C₁-C₁₀)-alkoxy-(C₁-C₁₀)-alkyl)-carbamoyloxy, N—(C₁-C₁₀)-alkyl-N—((C₆-C₁₂)-aryloxy-(C1-C₁₀)-alkyl)-carbamoyloxy, N—(C₁-C₁₀)-alkyl-N—((C₇-C₁₆)-aralkyloxy-(C₁-C₁₀)-alkyl)-carbamoyloxy, amino, (C₁-C₁₂)-alkylamino, di-(C₁-C₁₂)-alkylamino, (C₃-C₈)-cycloalkylamino, (C₂-C₁₂)-alkenylamino, (C₂-C₁₂)-alkynylamino, N—(C₆-C₁₂)-arylamino, N—(C-C₁₁)-aralkylamino, N-alkyl-aralkylamino, N-alkyl-arylamino, (C₁-C₁₂)-alkoxyamino, (C₁-C₁₂)-alkoxy-N—(C₁-C₁₀)-alkylamino, (C₁-C₁₂)-alkylcarbonylamino, (C₃-C₈)-cycloalkylcarbonylamino, (C₆-C₁₂) arylcarbonylamino, (C₇-C₁₆)-aralkylcarbonylamino, (C₁-C₁₂)-alkylcarbonyl-N—(C₁-C₁₀)-alkylamino, (C₃-C₈)-cycloalkylcarbonyl-N—(C₁-C₁₀)-alkylamino, (C₆-C₁₂)-arylcarbonyl-N—(C₁-C₁₀)alkylamino, (C₇-C₁₁)-aralkylcarbonyl-N—(C₁-C₁₀)-alkylamino, (C₁-C₁₂)-alkylcarbonylamino-(C₁-C₈)-alkyl, (C₃-C₈)-cycloalkylcarbonylamino-(C₁-C₈)alkyl, (C₆-C₁₂)-arylcarbonylamino-(C₁-C₈)-alkyl, (C₇-C₁₂)-aralkylcarbonylamino-(C₁-C₈)-alkyl, amino-(C₁-C₁₀)-alkyl, N—(C₁-C₁₀) alkylamino-(C₁-C₁₀)-alkyl, N,N-di-(C₁-C₁₀)-alkylamino-(C₁-C₁₀)-alkyl, (C₃-C₈)cycloalkylamino-(C₁-C₁₀)-alkyl, (C₁-C₁₂)-alkylmercapto, (C₁-C₁₂)-alkylsulfinyl, (C₁-C₁₂)-alkylsulfonyl, (C₆-C₁₆)-arylmercapto, (C₆-C₁₆)-arylsulfinyl, (C₆-C₁₂)-arylsulfonyl, (C₇-C₁₆)-aralkylmercapto, (C₇-C₁₆)-aralkylsulfinyl, C₇-C₁₆)-aralkylsulfonyl, sulfamoyl, N—(C₁-C₁₀)-alkylsulfamoyl, N,N-di(C₁-C₁₀)-alkylsulfamoyl, (C₃-C₈)-cycloalkylsulfamoyl, N—(C₆-C₁₂)-alkylsulfamoyl, N—(C₇-C₁₆)-aralkylsulfamoyl, N—(C₁-C₁₀)-alkyl-N—(C₆-C₁₂)-arylsulfamoyl, N—(C₁-C₁₀)-alkyl-N—(C₇-C₁₆)-aralkylsulfamoyl, (C₁-C₁₀)-alkylsulfonamido, N—((C₁-C₁₀)-alkyl)-(C₁-C₁₀)-alkylsulfonamido, (C₇-C₁₆)-aralkylsulfonamido, or N—((C₁-C₁₀)-alkyl-(C₇-C₁₆)-aralkylsulfonamido; wherein radicals which are aryl or contain an aryl moiety, may be substituted on the aryl by one to five identical or different hydroxyl, halogen, cyano, trifluoromethyl, nitro, carboxyl, (C₁-C₁₂)-alkyl, (C₃-C₈)-cycloalkyl, (C₆-C₁₂)-aryl, (C₇-C₁₆)-aralkyl, (C₁-C₁₂)-alkoxy, (C₁-C₁₂)-alkoxy-(C₁-C₁₂)alkyl, (C₁-C₁₂)-alkoxy-(C₁-C₁₂)alkoxy, (C₆-C₁₂)-aryloxy, (C₇-C₁₆)-aralkyloxy, (C₁-C₈)-hydroxyalkyl, (C₁-C₁₂)-alkylcarbonyl, (C₃-C₈)-cycloalkyl-carbonyl, (C₆-C₁₂)-arylcarbonyl, (C₇-C₁₆) aralkylcarbonyl, (C₁-C₁₂)-alkoxycarbonyl, (C₁-C₁₂)-alkoxy-(C₁-C₁₂)-alkoxycarbonyl, (C₆-C₁₂)-aryloxycarbonyl, (C₇-C₁₆)-aralkyloxycarbonyl, (C₃-C₈)-cycloalkoxycarbonyl, (C₂-C₁₂)-alkenyloxycarbonyl, (C₂-C₁₂)-alkynyloxycarbonyl, (C₁-C₁₂)-alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₁-C₁₂)-alkoxy-(C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N—(C₁-C₁₂)-alkylcarbamoyl, N,N-di-(C₁-C₁₂)-alkylcabamoyl, N—(C₃-C₈)-cycloalkylcarbamoyl, N—(C₆-C₁₂)-arylcarbamoyl, N—(C₇-C₁₆)-aralkylcarbamoyl, N—(C₁-C₁₀)-alkyl-N—(C₆-C₁₂)-arylcarbamoyl, N—(C₁-C₁₀)-alkyl-N—(C₇-C₁₆)-aralkylcarbamoyl, N—((C₁-C₁₀)-alkoxy-(C₁-C₁₀)-alkyl)-carbamoyl, N—((C₆-C₁₂)-aryloxy-(C₁-C₁₀)-alkyl)-carbamoyl, N—((C₇-C₁₆)-aralkyloxy-(C₁-C₁₀)-alkyl)-carbamoyl, N—(C₁-C₁₀)-alkyl-N—((C₁-C₁₀)-alkoxy-(C₁-C₁₀)-alkyl)-carbamoyl, N—(C₁-C₁₀)-alkyl-N—((C₆-C₁₂)-aryloxy-(C₁-C₁₀)-alkyl)-carbamoyl, N—(C₁-C₁₀)-alkyl-N—((C₇-C₁₆)-aralkyloxy-(C₁-C₁₀)-alkyl)-carbamoyl, carbamoyloxy, N—(C₁-C₁₂)-alkylcarbamoyloxy, N,N-di-(C₁-C₁₂)-alkylcarbamoyloxy, N—(C₃-C₈)-cycloalkylcarbamoyloxy, N—(C₆-C₁₂)-arylcarbamoyloxy, N—(C₇-C₁₆)-aralkylcarbamoyloxy, N—(C₁-C₁₀)-alkyl-N—(C₆-C₁₂)-arylcarbamoyloxy, N(C₁-C₁₀)-alkyl-N—(C₇-C₁₆)-aralkylcarbamoyloxy, N—((C₁-C₁₀)-alkyl)-carbamoyloxy, N—((C₆-C₁₂)-aryloxy-(C₁-C₁₀)-alkyl)-carbamoyloxy, N—((C₇-C₁₆)-aralkyloxy-(C₁-C₁₀)-alkyl)-carbamoyloxy, N—(C₁-C₁₀)-alkyl-N—((C₁-C₁₀)-alkoxy-(C₁-C₁₀)-alkyl)-carbamoyloxy, N—(C₁-C₁₀)-alkyl-N—((C₆-C₁₂)-aryloxy-(C₁-C₁₀)-alkyl)-carbamoyloxy, N—(C₁-C₁₀)-alkyl-N—((C₇-C₁₆)-aralkyloxy-(C₁-C₁₀)-alkyl)-carbamoyloxy, amino, (C₁-C₁₂)-alkylamino, di-(C₁-C₁₂)-alkylamino, (C₃-C₈)-cycloalkylamino, (C₃-C₁₂)-alkenylamino, (C₃-C₁₂)-alkynylamino, N—(C₆-C₁₂)-arylamino, N—(C₇-C₁₁)-aralkylamino, N-alkylaralkylamino, N-alkyl-arylamino, (C₁-C₁₂)-alkoxyamino, (C₁-C₁₂)-alkoxy-N—(C₁-C₁₀)-alkylamino, (C₁-C₁₂)-alkylcarbonylamino, (C₃-C₈)-cycloalkylcarbonylamino, (C₆-C₁₂)-arylcarbonylamino, (C₇-C₁₆)-alkylcarbonylamino, (C₁-C₁₂)-alkylcarbonyl-N—(C₁-C₁₀)-alkylamino, (C₃-C₈)-cycloalkylcarbonyl-N—(C₁-C₁₀)-alkylamino, (C₆-C₁₂)-arylcarbonyl-N—(C₁-C₁₀)-alkylamino, (C₇-C₁₁)-aralkylcarbonyl-N—(C₁-C₁₀)-alkylamino, (C₁-C₁₂)-alkylcarbonylamino-(C₁-C₈)-alkyl, (C₃-C₈)-cycloalkylcarbonylamino-(C₁-C₈)-alkyl, (C₆-C₁₂)-arylcarbonylamino-(C₁-C₈)-alkyl, (C₇-C₁₆)-aralkylcarbonylamino-(C₁-C₈)-alkyl, amino-(C₁-C₁₀)-alkyl, N—(C₁-C₁₀)-alkylamino-(C₁-C₁₀)-alkyl, N,N-di-(C₁-C₁₀)-alkylamino-(C₁-C₁₀)-alkyl, (C₃-C₈)-cycloalkylamino(C₁-C₁₀)-alkyl, (C₁-C₁₂)-alkylmercapto, (C₁-C₁₂)-alkylsulfinyl, (C₁-C₁₂)-alkylsulfonyl, (C₇-C₁₂)-arylmercapto, (C₆-C₁₂)-arylsulfinyl, C₆-C₁₂)-arylsulfonyl, (C₇-C₁₆)-aralkylmercapto, (C₇-C₁₆)-aralkylsulfinyl, or (C₇-C₁₆)-aralkylsulfonyl,
X is O or S;
Q is O, S, NR′, or a bond;
where, if Q is a bond, R⁴ is halogen, nitrile, or trifluoromethyl;
or where, if Q is O, S, or NR′, R⁴ is hydrogen, (C₁-C₁₀)-alkyl radical, (C₂-C₁₀)-alkenyl radical, (C₂-C₁₀)-alkynyl radical, wherein alkenyl or alkynyl radical contains one or two C—C multiple bonds; unsubstituted fluoroalkyl radical of the formula —[CH₂]ₓ—C_{f}H_{(2f+1-g)}—F_{g}, (C₁-C₈)-alkoxy-(C₁-C₆)-alkyl radical, (C₁-C₆)-alkoxy-(C₁-C₄)-alkoxy-(C₁-C₄)-alkyl radical, aryl radical, heteroaryl radical, (C₇-C₁₁)-aralkyl radical, or a radical of the formula Z
   —[CH₂]ᵥ—[O]_{w}—[CH₂]ₜ-E (Z)
   where
E is a heteroaryl radical, a (C₃-C₈)-cycloalkyl radical, or a phenyl radical of the formula F 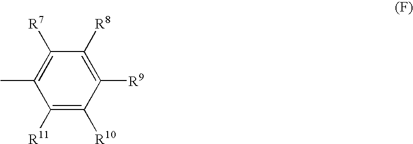
v is 0-6,
w is 0 or 1,
t is 0-3, and
R⁷, R⁸, R⁹, R¹⁰, and R¹¹ are identical or different and are hydrogen, halogen, cyano, nitro, trifluoromethyl, (C₁-C₆)-alkyl, (C₃-C₈)-cycloalkyl, (C₁-C₆)-alkoxy, —O—[CH₂]ₓ—C_{f}H_{(2f+1-g)}—F_{g}, —OCF₂—Cl, —O—CF₂—CHFCl, (C₁-C₆)-alkylmercapto, (C₁-C₆)-hydroxyalkyl, (C₁-C₆)-alkoxy-(C₁-C₆)-alkoxy, (C₁-C₆)-alkoxy-(C₁-C₆)-alkyl, (C₁-C₆)-alkylsulfinyl, (C₁-C₆)-alkylsulfinyl, (C₁-C₆)-alkylcarbonyl, (C₁-C₈)-alkoxycarbonyl, carbamoyl, N—(C₁-C₈)-alkylcarbamoyl, N,N-di-C₁-C₈)-alkylcarbamoyl, or (C₇-C₁₁)-aralkylcarbamoyl, optionally substituted by fluorine, chlorine, bromine, trifluoromethyl, (C₁-C₆)-alkoxy, N—(C₃-C₈)-cycloalkylcarbamoyl, N—(C₃-C₈)-cycloalkyl-(C₁-C₄)-alkylcarbamoyl, (C₁-C₆)-alkylcarbonyloxy, phenyl, benzyl, phenoxy, benzyloxy, NR^{Y}R^{Z} wherein R^{y} and R^{z} are independently selected from hydrogen, (C₁-C₁₂)-alkyl, (C₁-C₈)-alkoxy-(C₁-C₈)-alkyl, (C₇-C₁₂)-aralkoxy-(C₁-C₈)-alkyl, (C₆-C₁₂)-aryloxy-(C₁-C₈)-alkyl, (C₃-C₁₀)-cycloalkyl, (C₃-C₁₂)-aralkoxy-(C₁-C₈)-alkyl, (C₆-C₁₂)-aryloxy-(C₁-C₈)-alkyl, (C₃-C₁₀)-cycloalkyl, (C₃-C₁₂)aralkoxy, (C₁-C₁₂)-alkylcarbonyl, (C₃-C₈)-cycloalkylcarbonyl, (C₆-C₁₂) arylcarbonyl, (C₇-C₁₆)-aralkylcarbonyl; or further wherein R^{y} and R^{z} together are —[CH₂]ₕ, in which a CH₂ group can be replaced by O, S, N—(C₁-C₄)-alkylcarbonylimino, or N—(C₁-C₄)-alkoxycarbonylimino; phenylmercapto, phenylsulfonyl, phenylsulfinyl, sulfamoyl, N—(C₁-C₈)-alkylsulfamoyl, or N,N-di-(C₁-C₈)-alkylsulfamoyl; or alternatively R⁷ and R⁸, R⁸ and R⁹, R⁹ and R¹⁰, or R¹⁰ and R¹¹, together are a chain selected from —[CH₂]ₙ— or —CH═CH—CH═CH—, where a CH₂ group of the chain is optionally replaced by O, S, SO, SO₂, or NR^{Y}; and n is 3, 4, or 5; and if E is a heteroaryl radical, said radical can carry 1-3 substituents selected from those defined for R⁷—R¹¹, or if E is a cycloalkyl radical, the radical can carry one substituent selected from those defined for R⁷—R¹¹;
or where, if Q is NR′, R⁴ is alternatively R″, where R′ and R″ are identical or different and are hydrogen, (C₆-C₁₂)-aryl, (C₇-C₁₁)-aralkyl, (C₁-C₈)-alkyl, (C₁-C₈)-alkoxy-(C₁-C₈)-alkyl, (C₇-C₁₂)-(C₁-C₈)-alkyl, (C₆-C₁₂)-aryloxy-(C₁-C₈)-alkyl, (C₁-C₁₀)-alkylcarbonyl, optionally substituted (C₇-C₁₆)-aralkylcarbonyl, or optionally substituted C₆-C₁₂)-arylcarbonyl; or R′ and R″ together are - [CH₂]ₕ, in which a CH₂ group can be replaced by O, S, N-acylimino, or N—(C₁-C₁₀)-alkoxycarbonylimino, and h is 3 to 7.
Y is N or CR³;
R¹, R² and R³ are identical or different and are hydrogen, hydroxyl, halogen, cyano, trifluoromethyl, nitro, carboxyl, (C₁-C₂₀)-alkyl, (C₃-C₈)-cycloalkyl, (C₃-C₈)cycloalkyl-(C₁-C₁₂)-alkyl, (C₃-C₈)-cycloalkoxy, (C₃-C₈)-cycloalkyl-(C₁-C₁₂)-alkoxy, (C₃-C₈)-cycloalkyloxy-(C₁-C₁₂)-alkyl, (C₃-C₈)-cycloalkyloxy-(C₁-C₁₂)-alkoxy, (C₃-C₈)-cycloalkyl-(C₁-C₈)-alkyl-(C₁-C₆)-alkoxy, (C₃-C₈)-cycloalkyl-(C₁-C₈)-alkoxy-(C₁-C₆)-alkyl, (C₃-C₈)-cycloalkyloxy-(C₁-C₈)-alkoxy-(C₁-C₆)-alkyl, (C₃-C₈)-cycloalkoxy-(C₁-C₈)-alkoxy-(C₁-C₈)-alkoxy, (C₆-C₁₂)-aryl, (C₇-C₁₆)-aralkyl, (C₇-C₇-C₁₆)-aralkenyl, (C₇-C₁₆)-aralkynyl, (C₂-C₂₀)-alkenyl, (C₂-C₂₀)-alkynyl, (C₁-C₂₀)-alkoxy, (C₂-C₂₀)-alkenyloxy, (C₂-C₂₀)-alkynyloxy, retinyloxy, (C₁-C₂₀)-alkoxy-(C₁-C₁₂)-alkyl, (C₁-C₁₂)-alkoxy-(C₁-C₁₂)-alkoxy, (C₁-C₁₂)-alkoxy-(C₁-C₈)-alkoxy-(C₁-C₈)-alkyl, (C₆-C₁₂)-aryloxy, (C₇-C₁₆)-aralkyloxy, (C₆-C₁₂)-aryloxy-(C₁-C₆)-alkoxy, (C₇-C₁₆)-aralkoxy-(C₁-C₆)-alkoxy, (C₁-C₁₆)-hydroxyalkyl, (C₆-C₁₆)-aryloxy-(C₁-C₈)-alkyl, (C₇-C₁₆)-aralkoxy-(C₁-C₈)-alkyl, (C₆-C₁₂)-aryloxy-(C₁-C₈)-alkoxy-(C₁-C₆)-alkyl, (C₇-C₁₂)-aralkyloxy-(C₁-C₈)-alkoxy-(C₁-C₆)-alkyl, (C₂-C₂₀)-alkenyloxy-(C₁-C₆)-alkyl, (C₂-C₂₀)-alkynyloxy-(C₁-C₆)-alkyl, retinyloxy-(C₁-C₆)-alkyl, —O—CH₂]ₓC_{f}H_{(2f+1-g)}F_{g}, —OCF₂Cl, —OCF₂—CHFCl, (C₁-C₂₀)-alkylcarbonyl, (C₃-C₈)-cycloalkylcarbonyl, (C₆-C₁₂)-arylcarbonyl, (C₇-C₁₆)-alkylcarbonyl, cinnamoyl, (C₂-C₂₀)-alkenylcarbonyl, (C₂-C₂₀)-alkynylcarbonyl, (C₁-C₂₀)-alkoxycarbonyl, (C₁-C₁₂)-alkoxy-(C₁-C₁₂)-alkoxycarbonyl, (C₆-C₁₂)-aryloxycarbonyl, (C₇-C₁₆)-aralkyloxycarbonyl, (C₃-C₈)-cycloalkoxycarbonyl, (C₂-C₂₀)-alkenyloxycarbonyl, retinyloxycarbonyl, (C₂-C₂₀)-alkynyloxycarbonyl, (C₆-C₁₂)-aryloxy-(C₁-C₆)-alkoxycarbonyl, (C₇-C₁₆)-aralkoxy-(C₁-C₆)-alkoxycarbonyl, (C₃-C₈)-cycloalkyl-(C₁-C₆)-alkoxycarbonyl, (C₃-C₈)-cycloalkoxy-(C₁-C₆)-alkoxycarbonyl, (C₁-C₁₂)-alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₁-C₁₂)-alkoxy-(C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoy, N—(C₁-C₁₂)-alkylcarbamoyl, N,N-di-(C₁-C₁₂)-alkylcarbamoyl, N—(C₃-C₈)-cycloalkylcarbamoyl, N,N-dicyclo-(C₃-C₈)-alkylcarbamoyl, N—(C₁-C₁₀)-alkyl-N—(C₃-C₈)-cycloalkylcarbamoyl, N—((C₃-C₈)-cycloalkyl-(C₁-C₆)-alkyl)-carbamoyl, N—(C₁-C₆)-alkyl-N—((C₃-C₈)-cycloalkyl-(C₁-C₆)-alkyl)-carbamoyl, N-(+)-dehydroabietylcarbamoyl, N—(C₁-C₆)-alkyl-N-(+)-dehydroabietylcarbamoyl, N—(C₆-C₁₂)-arylcarbamoyl, N—(C₇-C₁₆)-aralkylcarbamoyl, N—(C₁-C₁₀)-alkyl-N—(C₆-C₁₆)-arylcarbamoyl, N—(C₁-C₁₀)-alkyl-N—(C₇-C₁₆)-aralkylcarbamoyl, N—((C₁-C₁₈)-alkoxy-C₁-C₁₀)-alkyl)-carbamoyl, N—((C₆-C₁₆)-aryloxy-(C₁-C₁₀)-alkyl)-alkoxy-(C₁-C₁₀)-alkyl)-carbamoyl, N—(C₁-C₁₀)-alkyl-N—((C₆-C₁₂)-aryloxy-(C₁-C₁₀)-alkyl)-N—((C₁-C₁₀)-carbamoyl, N—(C₁-C₁₀)-alkyl-N—((C₇-C₁₆)-aralkyloxy-(C₁-C₁₀)-alkyl)-carbamoyl; CON(CH₂)ₕ, in which a CH₂ group can be replaced by O, S, N—(C₁-C₈)-alkylimino, N—(C₃-C₈)-cycloalkylimino, N—(C₃-C₈)-cycloalkyl-(C₁-C₄)-alkylimino, N—(C₆-C₁₂)-arylimino, N—(C₇-C₁₆)-aralkylimino, N—(C₁-C₄)-alkoxy-(C₁-C₆)-alkylimino, and h is from 3 to 7; a carbamoyl radical of the formula R 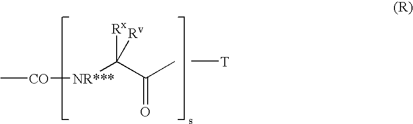
   in which
R^{x} and R^{v} are each independently selected from hydrogen, (C₁-C₆)-alkyl, (C₃-C₇)-cycloalkyl, aryl, or the substituent of an α-carbon of an α-amino acid, to which the L- and D-amino acids belong,
s is 1-5,
T is OH, or NR*R**, and R*, R** and R*** are identical or different and are selected from hydrogen, (C₆-C₁₂)-aryl, (C₇-C₁₁)-aralkyl, (C₁-C₈)-alkyl, (C₃-C₈)-cycloalkyl, (+)-dehydroabietyl, (C₁-C₈)-alkoxy-(C₁-C₈)-alkyl, (C₇-C₁₂)-aralkoxy-(C₁-C₈)-alkyl, (C₆-C₁₂)-aryloxy-(C₁-C₈)-alkyl, (C₁-C₁₀)-alkanoyl, optionally substituted (C₇-C₁₆)-aralkanoyl, optionally substituted (C₆-C₁₂)-aroyl; or R* and R** together are —[CH₂]ₕ, in which a CH₂ group can be replaced by O, S, SO, SO₂, N-acylamino, N—(C₁-C₁₀)-alkoxycarbonylimino, N—(C₁-C₈)-alkylimino, N—(C₃-C₈)-cycloalkylimino, N—(C₃-C₈)-cycloalkyl-(C₁-C₄)-alkylimino, N—(C₆-C₁₂)-arylimino, N—(C₇-C₁₆)-aralkylimino, N—(C₁-C₄)-alkoxy-(C₁-C₆)-alkylimino, and h is from 3 to 7; carbamoyloxy, N—(C₁-C₁₂)-alkylcarbamoyloxy, N,N-di-(C₁-C₁₂)-alkylcarbamoyloxy, N—(C₃-C₈)-cycloalkylcarbamoyloxy, N—(C₆-C₁₂)-arylcarbamoyloxy, N—(C₇-C₁₆)-aralkylcarbamoyloxy, N—(C₁-C₁₀)-alkyl-N—(C₆-C₁₂)-arylcarbamoyloxy, N—(C₁-C₁₀)-alkyl-N—(C₇-C₁₆)-aralkylcarbamoyloxy, N—((C₁-C₁₀)-alkyl)-carbamoyloxy, N—((C₆-C₁₂)-aryloxy-(C₁-C₁₀)-alkyl)-carbamoyloxy, N—((C₇-C₁₆)-aralkyloxy-(C₁-C₁₀)-alkyl)-carbamoyloxy, N—(C₁-C₁₀)-alkyl-N—((C₁-C₁₀)-alkoxy-(C₁-C₁₀)-alkyl)-carbamoyloxy, N—(C₁-C₁₀)-alkyl-N—((C₆-C₁₂)-aryloxy-(C₁-C₁₀)-alkyl)-carbamoyloxy, N—(C₁-C₁₀)-alkyl-N—((C₇-C₁₆)-aralkyloxy-(C₁-C₁₀)-alkyl)-carbamoyloxyamino, (C₁-C₁₂)-alkylamino, di-(C₁-C₁₂)-alkylamino, (C₃-C₈)-cycloalkylamino, (C₃-C₁₂)-alkenylamino, (C₃-C₁₂)-alkynylamino, N—(C₆-C₁₂)-arylamino, N—(C₇-C₁₁)-aralkylamino, N-alkyl-aralkylamino, N-alkyl-arylamino, (C₁-C₁₂)-alkoxyamino, (C₁-C₁₂)-alkoxy-N—(C₁-C₁₀)-alkylamino, (C₁-C₁₂)-alkanoylamino, (C₃-C₈)-cycloalkanoylamino, (C₆-C₁₂)-aroylamino, (C₇-C₁₆)-aralkanoylamino, (C₁-C₁₂)-alkanoyl-N—(C₁-C₁₀)-alkylamino, (C₃-C₈)-cycloalkanoyl-N—(C₁-C₁₀)-alkylamino, (C₆-C₁₂)-aroyl-N—(C₁-C₁₀)-alkylamino, (C₇-C₁₁)-aralkanoyl-N—(C₁-C₁₀)-alkylamino, (C₁-C₁₂)-alkanoylamino-(C₁-C₈)-alkyl, (C₃-C₈)-cycloalkanoylamino-(C₁-C₈)-alkyl, (C₆-C₁₂)-aroylamino-(C₁-C₈)-alkyl, (C₇-C₁₆)-aralkanoylamino-(C₁-C₈)-alkyl, amino-(C₁-C₁₀)-alkyl, N—(C₁-C₁₀)-alkylamino-(C₁-C₁₀)-alkyl, N,N-di(C₁-C₁₀)-alkylamino-(C₁-C₁₀)-alkyl, (C₃-C₈)-cycloalkylamino(C₁-C₁₀)-alkyl, (C₁-C₂₀)-alkylmercapto, (C₁-C₂₀)-alkylsulfinyl, (C₁-C₂₀)-alkylsulfonyl, (C₆-C₁₂)-arylmercapto, (C₆-C₁₂)-arylsulfinyl, (C₆-C₁₂)-arylsulfonyl, (C₇-C₁₆)-aralkylmercapto, (C₇-C₁₆)-aralkylsulfinyl, (C₇-C₁₆)-aralkylsulfonyl, (C₇-C₁₆)-aralkylsulfonyl, (C₁-C₁₂)-alkylmercapto-(C₁-C₆)-alkyl, (C₁-C₁₂)-alkylsulfinyl-(C₁-C₆)-alkyl, (C₁-C₁₂)-alkylsulfonyl-(C₁-C₆)-alkyl, (C₆-C₁₂)-arylmercapto-(C₁-C₆)-alkyl, (C₆-C₁₂)-arylsulfinyl-(C_{(C}₁-C₁₂)-alkylsulfonyl-(C₁-C₆)-alkyl, (C₆-C₁₂)-arylmercapto-(C₁-C₆)-alkyl, (C₆-C₁₂)-arylsulfinyl-(C₁-C₆)-alkyl, (C₆-C₁₂)-arylsulfonyl-(C₁-C₆)-alkyl, (C₇-C₁₆)-aralkylmercapto-(C₁-C₆)-alkyl, (C₇-C₁₆)-aralkylsulfinyl-(C₁-C₆)-alkyl, (C₇-C₁₆)-aralkylsulfonyl-(C₁-C₆)-alkyl, sulfamoyl, N—(C₁-C₁₀)-arylsulfamoyl, N,N-di-(C₁-C₁₀)-alkylsulfamoyl, (C₃-C₈)-cycloalkylsulfamoyl, N—(C₆-C₁₂)-arylsulfamoyl, N—(C₇-C₁₆)-aralkylsulfamoyl, N—(C₁-C₁₀)-alkyl-N—(C₆-C₁₂)-arylsulfamoyl, N—(C₁-C₁₀)-alkyl-N—(C₇-C₁₆)-aralkylsulfamoyl, (C₁-C₁₀)-alkylsulfonamido, N—((C₁-C₁₀)-alkyl)-(C₁-C₁₀)-alkylsulfonamido, (C₇-C₁₆)-aralkylsulfonamido, and N—((C₁-C₁₀)-alkyl-(C₇-C₁₆)- aralkylsulfonamido; where an aryl radical may be substituted by 1 to 5 substituents selected from hydroxyl, halogen, cyano, trifluoromethyl, nitro, carboxyl, (C₂-C₁₆)-alkyl, (C₃-C₈)-cycloalkyl, (C₃-C₈)-cycloalkyl-(C₁-C₁₂)-alkyl, (C₃-C₈)-cycloalkoxy, (C₃-C₈)-cycloalkyl-(C₁-C₁₂-alkoxy, (C₃-C₈)-cycloalkyloxy-(C₁-C₁₂)-alkyl, (C₃-C₈)-cycloalkyloxy-(C₁-C₁₂)-alkoxy, (C₃-C₈)-cycloalkyl-(C₁-C₈)-alkyl-(C₁-C₆)-alkoxy, (C₃-C₈)-cycloalkyl(C₁-C₈)-alkoxy-(C₁-C₆)-alkyl, (C₃-C₈)-cycloalkyloxy-(C₁-C₈)-alkoxy-(C₁-C₆)-alkyl, (C₃-C₈)-cycloalkoxy-(C₁-C₈)-alkoxy-(C₁-C₈)-alkoxy, (C₆-C₁₂)-aryl, (C₇-C₁₆)-aralkyl, (C₂-C₁₆)-alkenyl, (C₂-C₁₂)-alkynyl, (C₁-C₁₆)-alkoxy, (C₁-C₁₆)-alkenyloxy, (C₁c₁₂)-alkoxy-(C₁-C₁₂)-alkyl, (C₁-C₁₂)-alkoxy, (C₁-C₁₂)-alkoxy(C₁-C₈)-alkoxy-(C₁-C₈)-alkyl, (C₆-C₁₂)-aryloxy, (C₇-C₁₆)-aralkyloxy, (C₆-C₁₂)-aryloxy-(C₁-C₆)-alkoxy, (C₇-C₁₆)-aralkoxy-(C₁-C₆)-alkoxy, (C₁-C₈)-hydroxyalkyl, (C₆-C₁₆)-aryloxy-(C₁-C₈)-alkyl, (C₇-C₁₆)-aralkoxy-(C₁-C₈)-alkyl, (C₆-C₁₂)-aryloxy-(C₁-C₈)-alkoxy-(C₁-C₆)-alkyl, (C₇-C₁₂)-aralkyloxy-(C₁-C₈)-alkoxy-(C₁-C₆)-alkyl, —O—[CH₂]ₓC_{f}H_{(2f+1-g)}F_{g}, —OCF₂Cl, —OCF₂—CHFCl, (C₁-C₁₂)-alkylcarbonyl, (C₃-C₈)-cycloalkylcarbonyl, (C₆-C₁₂)-arylcarbonyl, (C₇-C₁₆)-aralkylcarbonyl, (C₁-C₁₂)-alkoxycarbonyl, (C₁-C₁₂)-alkoxy-(C₁-C₁₂)-alkoxycarbonyl, (C₆-C₁₂)-aryloxycarbonyl, (C₇-C₁₆)-aralkyloxycarbonyl, (C₃-C₈)-cycloalkoxycarbonyl, (C₂-C₁₂)-alkenyloxycarbonyl, (C₂-C₁₂)-alkynyloxycarbonyl, (C₆-C₁₂)-aryloxy-(C₁-C₆)-alkoxycarbonyl, (C₇-C₁₆)-aralkoxy-(C₁-C₆)-alkoxycarbonyl, (C₃-C₈)-cycloalkyl(C₁-C₆)-alkoxycarbonyl, (C₃-C₈)-cycloalkoxy-(C₁-C₆)-alkoxycarbonyl, (C₁-C₁₂)-alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₁-C₁₂)-alkoxy-(C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N—(C₁-C₁₂)-alkylcarbamoyl, N,N-di(C₁-C₁₂)-alkylcarbamoyl, N—(C₃-C₈)-cycloalkylcarbamoyl, N,N-dicyclo-(C₃-C₈)-alkylcarbamoyl, N—(C₁-C₁₀)-alkyl-N—(C₃-C₈)-cycloalkylcarbamoyl, N—((C₃-C₈)-cycloalkyl-(C₁-C₆)-alkyl)carbamoyl, N—(C₁-C₆)-alkyl-N—((C₃-C₈)-cycloalkyl-(C₁-C₆)-alkyl)carbamoyl, N-(+)-dehydroabietylcarbamoyl, N—(C₁-C₆)-alkyl-N-(+)-dehydroabietylcarbamoyl, N—(C₆-C₁₂)-arylcarbamoyl, N—(C₇-C₁₆)-aralkylcarbamoyl, N—(C₁-C₁₀)-alkyl-N—(C₆-C₁₆)-arylcarbamoyl, N—(C₁-C₁₀)-alkyl-N—(C₇-C₁₆)-aralkylcarbamoyl, N—((C₁-C₁₆)-alkoxy-(C₁-C₁₀)-alkyl)carbamoyl, N—((C₆-C₁₆)-aryloxy-(C₁-C₁₀)-alkyl)carbamoyl, N—((C₇-C₁₆)-aralkyloxy-(C₁-C₁₀)-alkyl)carbamoyl, N—(C₁-C₁₀)-alkyl-N—((C₁-C₁₀)-alkoxy-(C₁-C₁₀)-alkyl)carbamoyl, N—(C₁-C₁₀)-alkyl-N—((C₆-C₁₂)-aryloxy-(C₁-C₁₀)-alkyl)carbamoyl, N—(C₁-C₁₀)-alkyl-N—((C₇-C₁₆)-aralkyloxy-(C₁-C₁₀)-alkyl)-carbamoyl, CON(CH₂)h, in which a CH₂ group can be replaced by, O, S, N—(C₁-C₈)-alkylimino, N—(C₃-C₈)-cycloalkylimino, N—(C₃-C₈)-cycloalkyl-(C₁-C₄)-alkylimino, N—(C₆-C₁₂)-arylimino, N—(C₇-C₁₆)-aralkylimino, N—(C₁-C₄)-alkoxy-(C₁-C₆-alkylimino, and h is from 3 to 7; carbamoyloxy, N—(C₁-C₁₂)-alkylcarbamoyloxy, N,N-di-(C₁-C₁₂₎-alkylcarbamoyloxy, N—(C₃₋C₈₎-cycloalkylcarbamoyloxy, N—(C₆-C₁₆)-arylcarbamoyloxy,N—(C₇-C₁₆)-aralkylcarbamoyloxy, N—(C₁-C₁₀)-alkyl-N—(C₆-C₁₂)-arylcarbamoyloxy,N—(C₁-C₁₀)-alkyl-N—(C₇-C₁₆)-aralkylcarbamoyloxy, N—((C₁-C₁₀)-alkyl)carbamoyloxy, N—((C₆-C₁₂)-aryloxy-(C₁-C₁₀)-alkyl)carbamoyloxy, N—((C₇-C₁₆)-aralkyloxy-(C₁-C₁₀)-alkyl)carbamoyloxy,N—(C₁-C₁₀)-alkyl-N—((C₁-C₁₀)-alkoxy-(C₁-C₁₀)-alkyl)carbamoyloxy, N—(C₁-C₁₀)-alkyl-N—((C₆-C₁₂)-aryloxy-(C₁-C₁₀)-alkyl)carbamoyloxy, N—(C₁-C₁₀)-alkyl-N((C₇-C₁₆)-aralkyloxy-C₁-C₁₀)-alkyl)carbamoyloxy, amino, (C₁C₁₂)-alkylamino, di-(C₁-C₁₂)-alkylamino, (C₃-C₈)-cycloalkylamino, (C₃-C₁₂)-alkenylamino, (C₃-C₁₂)-alkynylamino, N—(C₆-C₁₂)-arylamino, N—(C₇-C₁₁)-aralkylamino, N-alkyl-aralkylamino, N-alkyl-arylamino, (C₁-C₁₂)-alkoxyamino, (C₁-C₁₂)-alkoxy-N—(C₁-C₁₀)- alkylamino, (C₁-C₁₂)-alkanoylamino, (C₃-C₈)-cycloalkanoylamino, (C₆-C₁₂)-aroylamino, (C₇-C₁₆)-aralkanoylamino, (C₁-C₁₂)-alkanoyl-N—(C₁-C₁₀)-alkylamino, (C₃-C₈)-cycloalkanoyl-N—(C₁-C₁₀)-alkylamino, (C₆-C₁₂)-aroyl-N—(C₁-C₁₀)-alkylamino, (C₇-C₁₁)-aralkanoyl-N—(C₁-C₁₀)-alkylamino, (C₁-C₁₂)-alkanoylamino-(C₁-C₈)-alkyl, (C₃-C₈)-cycloalkanoylamino-(C₁-C₈)-alkyl, (C₆-C₁₂)-aroylamino-(C₁-C₈)-alkyl, (C₇-C₁₆)-aralkanoylamino-(C₁-C₈)-alkyl, amino-(C₁-C₁₀)-alkyl, N—(C₁-C₁₀)-alkylamino-C₁-C₁₀)-alkyl, N,N-di-C₁-C₁₀)-alkylamino-C₁-C₁₀)-alkyl, (C₃-C₈)-cycloalkylamino-(C₁-C₁₀)-alkyl, (C₁-C₁₂)-alkylmercapto, (C₁-C₁₂)-alkylsulfinyl, (C₁-C₁₂)-alkylsulfonyl, (C₆-C₁₆)-arylmercapto, (C₆-C₁₆)-arylsulfinyl, (C₆-C₁₆)-arylsulfonyl, (C₇-C₁₆)-aralkylmercapto, (C₇-C₁₆)-aralkylsulfinyl, or (C₇-C₁₆)-aralkylsulfonyl;
or wherein R¹ and R², or R² and R³ form a chain [CH₂]ₒ, which is saturated or unsaturated by a C═C double bond, in which 1 or 2 CH₂ groups are optionally replaced by O, S, SO, SO₂, or NR′, and R′ is hydrogen, (C₆-C₁₂)-aryl, (C₁-C₈)-alkyl, (C₁-C₈)-alkoxy-(C₁-C₈)-alkyl, (C₇-C₁₂)-aralkoxy-(C₁-C₈)-alkyl, (C₆-C₁₂)-aryloxy-(C₁-C₈)-alkyl, (C₁-C₁₀)-alkanoyl, optionally substituted (C₇-C₁₆)-aralkanoyl, or optionally substituted (C₆-C₁₂)-aroyl; and o is 3, 4 or 5;
or wherein the radicals R¹ and R², or R² and R³, together with the pyridine or pyridazine carrying them, form a 5,6,7,8-tetrahydroisoquinoline ring, a 5,6,7,8-tetrahydroquinoline ring, or a 5,6,7,8-tetrahydrocinnoline ring;
or wherein R¹ and R², or R² and R³ form a carbocyclic or heterocyclic 5- or 6-membered aromatic ring;
or where R¹ and R², or R² and R³, together with the pyridine or pyridazine carrying them, form an optionally substituted heterocyclic ring systems selected from thienopyridines, furanopyridines, pyridopyridines, pyrimidinopyridines, imidazopyridines, thiazolopyridines, oxazolopyridines, quinoline, isoquinoline, and cinnoline; where quinoline, isoquinoline or cinnoline preferably satisfy the formulae Ia, Ib and Ic: 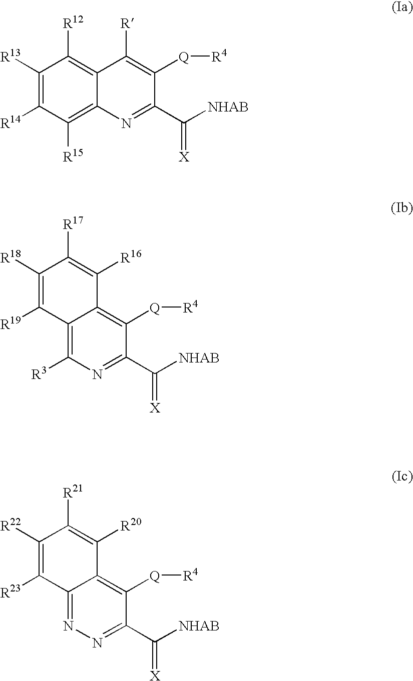
and the substituents R¹² to R²³ in each case independently of each other have the meaning of R¹, R²and R³;
or wherein the radicals R¹ and R², together with the pyridine carrying them, form a compound of Formula Id: 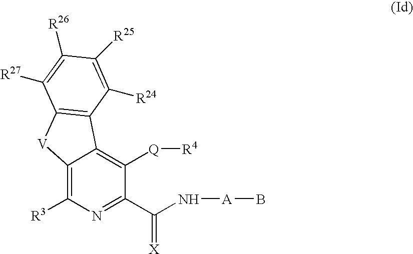
where V is S, O, or NR^{k}, and R^{k} is selected from hydrogen, (C₁-C₆)-alkyl, aryl, or benzyl;
where an aryl radical may be optionally substituted by 1 to 5 substituents as defined above; and
R²⁴, R²⁵, R²⁶, and R²⁷ in each case independently of each other have the meaning of R¹, R² and R³;
f is 1 to 8;
g is 0 or 1 to (2f+1);
x is 0 to 3; and
h is 3 to 7;
including the physiologically active salts and prodrugs derived therefrom.
Exemplary compounds according to Formula (I) are described in European Patent Nos. EP0650960 and EP0650961. All compounds listed in EP0650960 and EP0650961, in particular, those listed in the compound claims and the final products of the working examples, are hereby incorporated into the present application by reference herein. Exemplary compounds of Formula (I) include, but are not limited to, [(3-Hydroxy-pyridine-2-carbonyl)-amino]-acetic acid and [(3-methoxy-pyridine-2-carbonyl)-amino]-acetic acid.

Additionally, exemplary compounds according to Formula (I) are described in U.S. Pat. No. 5,658,933. All compounds listed in U.S. Pat. No. 5,658,933, in particular, those listed in the compound claims and the final products of the working examples, are hereby incorporated into the present application by reference herein. Exemplary compounds of Formula (I) include, but are not limited to, 3-methoxypyridine-2-carboxylic acid N-(((hexadecyloxy)-carbonyl)-methyl)-amide hydrochloride, 3-methoxypyridine-2-carboxylic acid N-(((1-octyloxy)-carbonyl)-methyl)-amide, 3-methoxypyridine-2-carboxylic acid N-(((hexyloxy)-carbonyl)-methyl)-amide, 3-methoxypyridine-2-carboxylic acid N-(((butyloxy)-carbonyl)-methyl)-amide, 3-methoxypyridine-2-carboxylic acid N-(((2-nonyloxy)-carbonyl)-methyl)-amide racemate, 3-methoxypyridine-2-carboxylic acid N-(((heptyloxy)-carbonyl)-methyl)-amide, 3-benzyloxypyridine-2-carboxylic acid N-(((octyloxy)-carbonyl)-methyl)-amide, 3-benzyloxypyridine-2-carboxylic acid N-(((butyloxy)-carbonyl)-methyl)-amide, 5-(((3-(1-butyloxy)-propyl)-amino)-carbonyl)-3-methoxypyridine-2-carboxylic acid N-((benzyloxycarbonyl)-methyl)-amide, 5-(((3-(1butyloxy)-propyl)-amino)-carbonyl)-3-methoxypyridine-2-carboxylic acid N-(((1-butyloxy)-carbonyl)-methyl)-amide, and 5-(((3-lauryloxy)-propyl)amino)-carbonyl)-3-methoxypyridine-2-carboxylic acid N-(((benzyloxy)-carbonyl)-methyl)-amide.

Additional compounds according to Formula (I) are substituted heterocyclic carboxyamides described in U.S. Pat. No. 5,620,995; 3-hydroxypyridine-2-carboxamidoesters described in U.S. Pat. No. 6,020,350; sulfonamidocarbonylpyridine-2-carboxamides described in U.S. Pat. No. 5,607,954; and sulfonamidocarbonyl-pyridine-2-carboxamides and sulfonamidocarbonyl-pyridine-2-carboxamide esters described in U.S. Pat. Nos. 5,610,172 and 5,620,996. All compounds listed in these patents, in particular, those compounds listed in the compound claims and the final products of the working examples, are hereby incorporated into the present application by reference herein.
Exemplary compounds according to Formula (Ia) are described in U.S. Pat. Nos. 5,719,164 and 5,726,305. All compounds listed in the foregoing patents, in particular, those listed in the compound claims and the final products of the working examples, are hereby incorporated into the present application by reference herein. Exemplary compounds of Formula (Ia) include, but are not limited to, N-((3-hydroxy-6-isopropoxy-quinoline-2-carbonyl)-amino)-acetic acid, N-((6-(1-butyloxy)-3-hydroxyquinolin-2-yl)-carbonyl)-glycine, [(3-hydroxy-6-trifluoromethoxy-quinoline-2-carbonyl)-amino]-acetic acid, N-((6-Chloro-3-hydroxyquinolin-2-yl)-carbonyl)-glycine, N-((7-Chloro-3-hydroxyquinolin-2-yl)-carbonyl)-glycine, and [(6-Chloro-3-hydroxy-quinoline-2-carbonyl)-amino]-acetic acid.

Exemplary compounds according to Formula (Ib) are described in U.S. Pat. No. 6,093,730. All compounds listed in U.S. Pat. No. 6,093,730, in particular, those listed in the compound claims and the final products of the working examples, are hereby incorporated into the present application by reference herein. Exemplary compounds of Formula (1b) include, but are not limited to, N-((1-chloro-4-hydroxy-7-(2-propyloxy) isoquinolin-3-yl)-carbonyl)-glycine, N-((1-chloro-4-hydroxy-6-(2-propyloxy) isoquinolin-3-yl)-carbonyl)-glycine, N-((1-Chloro-4-hydroxy-isoquinoline-3-carbonyl)-amino)-acetic acid (compound A), N-((1-Chloro-4-hydroxy-7-methoxyisoquinolin-3-yl)-carbonyl)-glycine, N-((1-Chloro-4-hydroxy-6-methoxyisoquinolin-3-yl)-carbonyl)-glycine, N-((7-butyloxy)-1-Chloro-4-hydroxyisoquinolin-3-yl)-carbonyl)-glycine, N-((6-benzyloxy-1-Chloro-4-hydroxy-isoquinoline-3-carbonyl)-amino)-acetic acid, ((7-benzyloxy-1-Chloro-4-hydroxy-isoquinoline-3-carbonyl)-amino)-acetic acid methyl ester, N-((7-benzyloxy-1-Chloro-4-hydroxy-isoquinoline-3-carbonyl)-amino)-acetic acid, N-((8-Chloro-4-hydroxyisoquinolin-3-yl)-carbonyl)-glycine, N-((7-butoxy-4-hydroxy-isoquinoline-3-carbonyl)-amino)-acetic acid.

Additionally, compounds related to Formula (I) that can also be used in the methods of the invention include, but are not limited to, 6-cyclohexyl-1-hydroxy-4-methyl-1H-pyridin-2-one, 7-(4-methyl-piperazin-1-ylmethyl)-5-phenylsulfanylmethyl-quinolin-8-ol, 4-nitro-quinolin-8-ol, 5-butoxymethyl-quinolin-8-ol, [(4-Hydroxy-7-phenoxy-isoquinoline-3-carbonyl)-amino]-acetic acid (compound B), and [(4-Hydroxy-7-phenylsulfanyl-isoquinoline-3-carbonyl)-amino]-acetic acid (compound C). Further, the invention provides additional exemplary compounds wherein, e.g., position A and B together may be, e.g., hexanoic acid, cyanomethyl, 2-aminoethyl, benzoic acid, 1H-benzoimidazol-2-ylmethyl, etc.

In other embodiments, compounds used in the methods of the invention are selected from a compound of the formula (III) 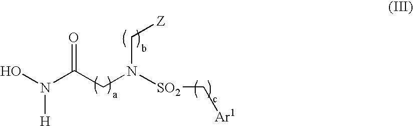
or pharmaceutically acceptable salts thereof, wherein:
a is an integer from 1 to 4;
b is an integer from 0 to 4;
c is an integer from 0 to 4;
Z is selected from the group consisting of (C₃-C₁₀) cycloalkyl, (C₃-C₁₀) cycloalkyl independently substituted with one or more Y¹, 3-10 membered heterocycloalkyl and 3-10 membered heterocycloalkyl independently substituted with one or more Y¹; (C₅-C₂₀) aryl, (C₅-C₂₀) aryl independently substituted with one or more Y¹, 5-20 membered heteroaryl and 5-20 membered heteroaryl independently substituted with one or more Y¹;

Ar¹ is selected from the group consisting of (C₅-C₂₀) aryl, (C₅-C₂₀) aryl independently substituted with one or more Y², 5-20 membered heteroaryl and 5-20 membered heteroaryl independently substituted with one or more Y²;
each Y¹ is independently selected from the group consisting of a lipophilic functional group, (C₅-C₂₀) aryl, (C₆-C₂₆) alkaryl, 5-20 membered heteroaryl and 6-26 membered alk-heteroaryl;
each Y² is independently selected from the group consisting of —R′, —OR′, —OR″, —SR′, —SR″, —NR′R′, —NO₂, —CN, -halogen, -trihalomethyl, trihalomethoxy, —C(O)R′, —C(O)OR′, —C(O)NR′R′, —C(O)NR′OR′, —C(NR′R′)═NOR′, —NR′—C(O)R′, —SO₂R′, —SO₂R″, —NR′—SO₂—R′, —NR′—C(O)—NR′R′, tetrazol-5-yl, —NR′—C(O)—OR′, —C(NR′R′)═NR′, —S(O)—R′, —S(O)—R″, and —NR′—C(S)—NR′R′; and
each R′ is independently selected from the group consisting of —H, (C₁-C₈) alkyl, (C₂-C8) alkenyl, and (C₂-C₈) alkynyl; and
each R″ is independently selected from the group consisting of (C₅-C₂₀) aryl and (C₅-C₂₀) aryl independently substituted with one or more —OR′, —SR′, —NR′R′, —NO₂, —CN, halogen or trihalomethyl groups,
or wherein c is 0 and Ar¹ is an N′ substituted urea-aryl, the compound has the structural formula (IIIa): 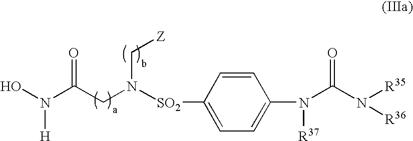
or pharmaceutically acceptable salts thereof, wherein:
a, b, and Z are as defined above; and
R³⁵ and R³⁶ are each independently selected from the group consisting of hydrogen, (C₁-C₈) alkyl, (C₂-C₈) alkenyl, (C₂-C₈) alkynyl, (C₃-C₁₀) cycloalkyl, (C₅-C₂₀) aryl, (C₅-C₂₀) substituted aryl, (C₆-C₂₆) alkaryl, (C₆-C₂₆) substituted alkaryl, 5-20 membered heteroaryl, 5-20 membered substituted heteroaryl, 6-26 membered alk-heteroaryl, and 6-26 membered substituted alk-heteroaryl; and
R³⁷ is independently selected from the group consisting of hydrogen, (C₁-C₈) alkyl, (C₂-C₈) alkenyl, and (C₂-C₈) alkynyl.

Exemplary compounds of Formula (III) are described in International Publication No. WO 00/50390. All compounds listed in WO 00/50390, in particular, those listed in the compound claims and the final products of the working examples, are hereby incorporated into the present application by reference herein. Exemplary compounds of Formula (III) include 3-{[4-(3,3-dibenzyl-ureido)-benzenesulfonyl]-[2-(4-methoxy-phenyl)-ethyl]-amino}-N-hydroxy-propionamide), 3-{{4-[3-(4-Chloro-phenyl)-ureido]-benzenesulfonyl}-[2-(4-methoxy-phenyl)-ethyl]-amino}-N-hydroxy-propionamide, and 3-{{4-[3-(1,2-diphenyl-ethyl)-ureido]-benzenesulfonyl}-[2-(4-methoxy-phenyl)-ethyl]-amino}-N-hydroxy-propionamide.

Methods for identifying compounds of the invention are also provided. In certain aspects, a compound of the invention is one that stabilizes HIFα. The ability of a compound to stabilize or activate HIFα can be measured, for example, by direct measurement of HIFα in a sample, indirect measurement of HIFα, e.g., by measuring a decrease in HIFα associated with the von Hippel Lindau protein (see, e.g., International Publication No. WO 00/69908), or activation of HIF responsive target genes or reporter constructs (see, e.g., U.S. Pat. No. 5,942,434). Measuring and comparing levels of HIF and/or HIF-responsive target proteins in the absence and presence of the compound will identify compounds that stabilize HIFα and/or activate HIF.

In other aspects, a compound of the invention is one that inhibits HIF hydroxylase activity. Assays for hydroxylase activity are standard in the art. Such assays can directly or indirectly measure hydroxylase activity. For example, an assay can measure hydroxylated residues, e.g., proline, asparagine, etc., present in the enzyme substrate, e.g., a target protein, a synthetic peptide mimetic, or a fragment thereof. (See, e.g., Palmerini et al. (1985) J Chromatogr 339:285-292.) A reduction in hydroxylated residue, e.g., proline or asparagine, in the presence of a compound is indicative of a compound that inhibits hydroxylase activity. Alternatively, assays can measure other products of the hydroxylation reaction, e.g., formation of succinate from 2-oxoglutarate. (See, e.g., Cunliffe et al. (1986) Biochem J 240:617-619.) Kaule and Gunzler (1990; Anal Biochem 184:291-297) describe an exemplary procedure that measures production of succinate from 2-oxoglutarate.

Procedures such as those described above can be used to identify compounds that modulate HIF hydroxylase activity. Target protein may include HIFα or a fragment thereof, e.g., HIF(556-575). Enzyme may include, e.g., HIF prolyl hydroxylase (see, e.g., GenBank Accession No. AAG33965, etc.) or HIF asparaginyl hydroxylase (see, e.g., GenBank Accession No. AAL27308, etc.), obtained from any source. Enzyme may also be present in a crude cell lysate or in a partially purified form. For example, procedures that measure HIF hydroxylase activity are described in Ivan et al. (2001, Science 292:464-468; and 2002, Proc Natl Acad Sci USA 99:13459-13464) and Hirsila et al. (2003, J Biol Chem 278:30772-30780); additional methods are described in International Publication No. WO 03/049686. Measuring and comparing enzyme activity in the absence and presence of the compound will identify compounds that inhibit hydroxylation of HIFα.

A compound of the invention is one that further produces a measurable effect, as measured in vitro or in vivo, as demonstrated by enhanced erythropoiesis, enhanced iron metabolism, or therapeutic improvement of conditions including, e.g., iron deficiency, including functional iron deficiency; anemia of chronic disease, iron deficiency, and microcytosis or microcytic anemia; or a condition associated with inflammation, infection, immunodeficiency, or neoplastic disorder.

The measurable effect can be any one of the following parameters: increased hemoglobin, hematocrit, reticulocyte, red blood cell count, plasma EPO, etc.; improved iron metabolism, as measured by lessening of observed symptoms, including, e.g., mitigation of chronic fatigue, pallor, dizziness, etc., or by increased serum iron levels, altered serum ferritin levels, % transferrin saturation, total iron binding capacity, improved reticulocyte counts, hemoglobin, hematocrit, e.g., all as measured by standard blood count analysis.

Preferred Compounds

In a particularly preferred embodiment, the compounds used in the present invention are as disclosed in WO 2004/108681, represented by formula (IV): 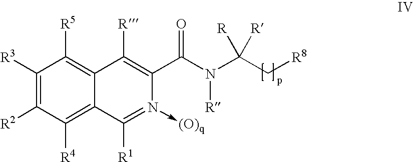
wherein:
q is zero or one;
p is zero or one;
R^{a} is —COOH or —WR⁸; provided that when R^{a} is —COOH then p is zero and when R^{a} is —WR⁸ then p is one;
W is selected from the group consisting of oxygen, —S(O)ₙ— and —NR⁹— where n is zero, one or two, R⁹ is selected from the group consisting of hydrogen, alkyl, substituted alkyl, acyl, aryl, substituted aryl, heteroaryl, substituted heteroaryl, heterocyclic and substituted heterocyclic and R⁸ is selected from the group consisting of hydrogen, alkyl, substituted alkyl, aryl, substituted aryl, heteroaryl, substituted heteroaryl, heterocyclic and substituted heterocyclic, or when W is —NR⁹— then R⁸ and R⁹, together with the nitrogen atom to which they are bound, can be joined to form a heterocyclic or a substituted heterocyclic group, provided that when W is —S(O)ₙ— and n is one or two, then R⁸ is not hydrogen;
R¹ is selected from the group consisting of hydrogen, alkyl, substituted alkyl, alkoxy, substituted alkoxy, amino, substituted amino, aminoacyl, aryl, substituted aryl, halo, heteroaryl, substituted heteroaryl, heterocyclic, substituted heterocyclic, and —XR⁶ where X is oxygen, —S(O)ₙ— or —NR⁷— where n is zero, one or two, R⁶ is selected from the group consisting of alky, substituted alkyl, aryl, substituted aryl, heteroaryl, substituted heteroaryl, heterocyclic and substituted heterocyclic, and R⁷ is hydrogen, alkyl or aryl or, when X is —NR⁷—, then R⁷ and R⁸, together with the nitrogen atom to which they are bound, can be joined to form a heterocyclic or substituted heterocyclic group;
R² and R³ are independently selected from the group consisting of hydrogen, alkyl, substituted alkyl, aryl, substituted aryl, heteroaryl, substituted heteroaryl, halo, hydroxy, cyano, —S(O)ₙ—N(R⁶)—R⁶ where n is 0, 1, or 2, —NR⁶C(O)NR⁶R⁶, —XR⁶ where X is oxygen, —S(O)ₙ— or —NR⁷— where n is zero, one or two, each R⁶ is independently selected from the group consisting of hydrogen, alkyl, substituted alkyl, aryl, substituted aryl, cycloalkyl, substituted cycloalkyl, heteroaryl, substituted heteroaryl, heterocyclic and substituted heterocyclic provided that when X is —SO— or —SO₂—, then R₆ is not hydrogen, and R⁷ is selected from the group consisting of hydrogen, alkyl, aryl, or R², R³ together with the carbon atom pendent thereto, form an aryl substituted aryl, heteroaryl, or substituted heteroaryl;
R⁴ and R⁵ are independently selected from the group consisting of hydrogen, halo, alkyl, substituted alkyl, alkoxy, substituted alkoxy, aryl, substituted aryl, heteroaryl, substituted heteroaryl and —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl or, when X is —NR⁷—, then R⁷ and R⁸, together with the nitrogen atom to which they are bound, can be joined to form a heterocyclic or substituted heterocyclic group;
R is selected from the group consisting of hydrogen, deuterium and methyl;
R′ is selected from the group consisting of hydrogen, deuterium, alkyl and substituted alkyl; alternatively, R and R′ and the carbon pendent thereto can be joined to form cycloalkyl, substituted cycloalkyl, heterocyclic or substituted heterocyclic group;
R″ is selected from the group consisting of hydrogen and alkyl or R″ together with R′ and the nitrogen pendent thereto can be joined to form a heterocyclic or substituted heterocyclic group;
R′″ is selected from the group consisting of hydroxy, alkoxy, substituted alkoxy, acyloxy, cycloalkoxy, substituted cycloalkoxy, aryloxy, substituted aryloxy, heteroaryloxy, substituted heteroaryloxy, aryl, —S(O)ₙ—R¹⁰ wherein R¹⁰ is selected from the group consisting of alkyl, substituted alkyl, cycloalkyl, substituted cycloalkyl, aryl, substituted aryl, heteroaryl and substituted heteroaryl and n is zero, one or two;
and pharmaceutically acceptable salts, esters and prodrugs thereof.

In particular embodiments, the compounds used in the present invention are represented by formula (IV) as described above with the proviso that when R, R′ and R″ are hydrogen and q is zero, and R^{a} is either —COOH (p is zero) or —WR⁸ (p is one) and W is oxygen and R⁸ is hydrogen then at least one of the following occurs:
1) R¹ is fluoro, bromo, iodo, alkyl, substituted alkyl, alkoxy, aminoacyl, substituted alkoxy, aryl, substituted aryl, heteroaryl, substituted heteroaryl, heterocyclic, substituted heterocyclic, and —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl; or
2) R² is substituted alkyl, aryl, substituted aryl, heteroaryl, substituted heteroaryl, fluoro, bromo, iodo, cyano, —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl provided that:
   a) when R² is substituted alkyl such a substituent does not include trifluoromethyl;
   b) —XR⁶ is not alkoxy; and
   c) when —XR⁶ is substituted alkoxy such a substituent does not include benzyl or benzyl substituted by a substituent selected from the group consisting of (C₁-C₅) alkyl and (C₁-C₅) alkoxy or does not include a fluoroalkoxy substituent of the formula:
      —O—[CH₂]ₓ—C_{f}H_{(2f+1-g)}F_{g}
where x is zero or one; f is an integer of from 1 to 5; and g is an integer of from 1 to (2f +1); or
3) R³ is substituted alkyl, aryl, substituted aryl, heteroaryl, substituted heteroaryl, bromo, iodo, —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl provided that:
   a) when R³ is substituted alkyl such a substituent does not include trifluoromethyl;
   b) —XR⁶ is not alkoxy; and
   c) when —XR⁶ is substituted alkoxy such a substituent does not include benzyl or benzyl substituted by a substituent selected from the group consisting of (C₁-C₅) alkyl and (C₁-C₅) alkoxy or does not include a fluoroalkoxy substituent of the formula:
      —O—[CH₂]ₓ—C_{f}H_{(2f+1-g)}F_{g}
where x is zero or one; f is an integer of from 1 to 5; and g is an integer of from 1 to (2f+1); or
4) R⁴ is iodo, substituted alkyl, aryl, substituted aryl, heteroaryl, substituted heteroaryl, —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl provided that:
   a) when R⁴ is substituted alkyl such a substituent does not include trifluoromethyl;
   b) —XR⁶ is not alkoxy; and
   c) when —XR⁶ is substituted alkoxy such a substituent does not include a fluoroalkoxy substituent of the formula:
      —O—[CH₂]ₓ—C_{f}H_{(2f+1-g)}F_{g}
where x is zero or one; f is an integer of from 1 to 5; and g is an integer of from 1 to (2f+1); or
5) R⁵ is iodo, substituted alkyl, aryl, substituted aryl, heteroaryl, substituted heteroaryl, —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl provided that:
   a) when R⁵ is substituted alkyl such a substituent does not include trifluoromethyl;
   b) —XR⁶ is not alkoxy; and
   c) when —XR⁶ is substituted alkoxy such a substituent does not include a fluoroalkoxy substituent of the formula:
      —O—[CH₂]ₓ—C_{f}H_{(2f+1-g)}F_{g}
where x is zero or one; f is an integer of from 1 to 5; and g is an integer of from 1 to (2f+1);
and with the further following proviso: that when R¹, R³, R⁴, and R⁵ are hydrogen, then R² is not bromo.

In an alternative embodiment, the compounds of formula (IV) are represented by formula (IVA): 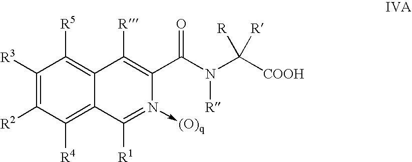
wherein R¹, R²,R, R⁴, R⁵, R, R′, R″, R′″ and q are as defined above; and pharmaceutically acceptable salts, esters, prodrugs thereof.

In an another alternative embodiment, the compounds of formula (IV) are represented by the formula (IVB): 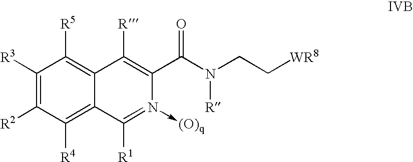
wherein R¹, R², R³, R⁴, R⁵, R″, R′″, WR⁸ and q are as defined above; and pharmaceutically acceptable salts, esters, prodrugs thereof.

In an another alternative embodiment, the invention is directed to compounds represented by the formula (IVC): 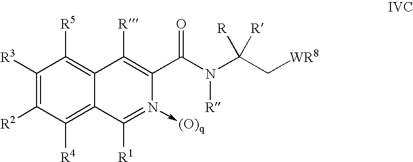
wherein R¹, R², R³, R⁴, R⁵, R, R′, R″, R′″, WR⁸ and q are as defined above; and pharmaceutically acceptable salts, esters, prodrugs thereof.

In yet another alternative embodiment, the invention is directed to compounds represented by the formula (IVD): 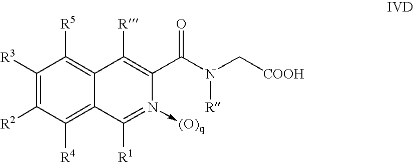
wherein R¹, R², R³, R⁴, R⁵, R, R′, R″, R′″ and q are as defined above; and pharmaceutically acceptable salts, esters, prodrugs thereof.

In other embodiments, the invention is directed to compounds represented by the formulae (VA), (VB), (VC), (VD), wherein said formulae are defined below.

Formula VA: 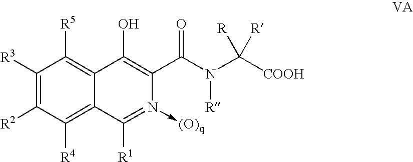
wherein:
q is zero or one;
R¹ is selected from the group consisting of hydrogen, alkyl, substituted alkyl, alkoxy, substituted alkoxy, aryl, substituted aryl, halo, heteroaryl, substituted heteroaryl, heterocyclic, substituted heterocyclic, and —XR⁶ where X is oxygen, —S(O)ₙ— or —NR⁷— where n is zero, one or two, R⁶ is selected from the group consisting of alkyl substituted alkyl, aryl, substituted aryl, heteroaryl, substituted heteroaryl, heterocyclic and substituted heterocyclic, and R⁷ is hydrogen, alkyl or aryl;
R² and R³ are independently selected from the group consisting of hydrogen, alkyl, substituted alkyl, alkoxy, substituted alkoxy, aryl, substituted aryl, heteroaryl, substituted heteroaryl, halo, hydroxy, cyano, —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl;
R⁴ and R⁵ are independently selected from the group consisting of hydrogen, halo, alkyl, substituted alky, alkoxy, substituted alkoxy, aryl, substituted aryl, heteroaryl, substituted heteroaryl and —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl;
R is selected from the group consisting of hydrogen and methyl;
R′ is selected from the group consisting of alkyl and substituted alkyl; or R and R′ may be joined to form a cycloalkyl, substituted cycloalkyl, heterocyclic or substituted heterocyclic; and
R″ is selected from the group consisting of hydrogen and alkyl or R″ together with R′ and the nitrogen pendent thereto forms a heterocyclic or substituted heterocyclic group;
or pharmaceutically acceptable salts and/or prodrugs thereof

Formula VB: 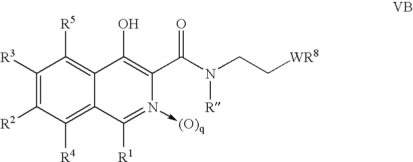
wherein:
q is zero or one;
W is selected from the group consisting of oxygen, —S(O)ₙ— and —NR⁹— where n is zero, one or two, R⁹ is selected from the group consisting of hydrogen, alkyl, substituted alkyl, acyl, aryl, substituted aryl, heteroaryl, substituted heteroaryl, heterocyclic and substituted heterocyclic, and R⁸ is selected from the group consisting of hydrogen, alkyl, substituted alkyl, aryl, substituted aryl, heteroaryl, substituted heteroaryl, heterocyclic and substituted heterocyclic;
R″ is selected from hydrogen and alkyl;
R′ is selected from the group consisting of hydrogen, alkyl, substituted alkyl, alkoxy, substituted alkoxy, aryl, substituted aryl, halo, heteroaryl, substituted heteroaryl, heterocyclic, substituted heterocyclic, and —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl;
R² and R³ are independently selected from the group consisting of hydrogen, alkyl, substituted alkyl, alkoxy, substituted alkoxy, aryl, substituted aryl, heteroaryl, substituted heteroaryl, halo, hydroxy, cyano, —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl; and
R⁴ and R⁵ are independently selected from the group consisting of hydrogen, halo, alkyl, substituted alkyl, alkoxy, substituted alkoxy, aryl, substituted aryl, heteroaryl, substituted heteroaryl and —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl;
or pharmaceutically acceptable salts and/or prodrugs thereof.

Formula VC: 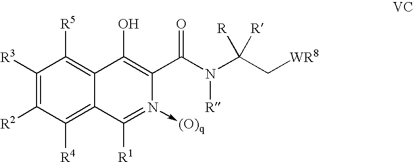
wherein:
q is zero or one;
R¹ is selected from the group consisting of hydrogen, alkyl, substituted alkyl, alkoxy, substituted alkoxy, aryl, substituted aryl, halo, heteroaryl, substituted heteroaryl, heterocyclic, substituted heterocyclic, and —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl, or aryl;
R² and R³ are independently selected from the group consisting of hydrogen, alkyl, substituted alkyl, alkoxy, substituted alkoxy, aryl, substituted aryl, heteroaryl, substituted heteroaryl, halo, hydroxy, cyano, XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷is hydrogen, alkyl, or aryl;
R⁴ and R⁵ are independently selected from the group consisting of hydrogen, halo, alkyl, substituted alkyl, alkoxy, substituted alkoxy, aryl, substituted aryl, heteroaryl, substituted heteroaryl and —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷is hydrogen, alkyl, or aryl;
R is selected from the group consisting of hydrogen and methyl;
R′ is selected from the group consisting of alkyl and substituted alkyl; or R and R′ can be joined to form cycloalkyl, substituted cycloalkyl, heterocyclic or substituted heterocyclic
R″ is selected from the group consisting of hydrogen and alkyl or R″ together with R′ and the nitrogen pendent thereto forms a heterocyclic or substituted heterocyclic group; and

W is selected from the group consisting of oxygen, —S(O)ₙ— and —NR⁹— where n is zero, one or two, R⁹ is selected from the group consisting of hydrogen, alkyl, substituted alkyl, aryl, substituted aryl, heteroaryl, substituted heteroaryl, heterocyclic and substituted heterocyclic, and R⁸ is selected from the group consisting of hydrogen, alkyl, substituted alkyl, aryl, substituted aryl, heteroaryl, substituted heteroaryl, heterocyclic and substituted heterocyclic;
or pharmaceutically acceptable salts and/or prodrugs thereof.

Formula VD: 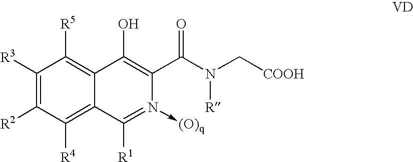
wherein:
q is zero or one;
R″ is selected from hydrogen and alkyl;
R¹ is selected from the group consisting of hydrogen, alkyl, substituted alkyl, alkoxy, substituted alkoxy, aryl, substituted aryl, halo, heteroaryl, substituted heteroaryl, heterocyclic, substituted heterocyclic, and —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl;
R² and R³ are independently selected from the group consisting of hydrogen, alkyl, substituted alkyl, aryl, substituted aryl, heteroaryl, substituted heteroaryl, halo, hydroxy, cyano, —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl; and
R⁴ and R⁵ are independently selected from the group consisting of hydrogen, halo, alkyl, substituted alkyl, alkoxy, substituted alkoxy, aryl, substituted aryl, heteroaryl, substituted heteroaryl and —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl;
or pharmaceutically acceptable salts and/or prodrugs thereof.

In compounds of formulae (IV), (IVA), (IVB), (IVC), and (IVD), preferably R¹ is selected from the group consisting of hydrogen, alkyl, substituted alkyl, halo, alkoxy, aryloxy, substituted aryloxy, 5 substituted aryl, alkylthio, aminoacyl, aryl, substituted amino, heteroaryl, heteroaryloxy, —S(O)ₙ-aryl, —S(O)ₙ-substituted aryl, —S(O)ₙ-heteroaryl, and —S(O)ₙ-substituted heteroaryl, where n is zero, one or two.

More preferably, R¹ is selected from the group consisting of (3-methoxyphenyl)sulfanyl; (4-chlorophenyl)sulfanyl; (4-methylphenyl)sulfanyl; 2-fluorophenoxy; 2-methoxyphenoxy; (2-methoxyphenyl)sulfanyl3-fluorophenoxy; 3-methoxyphenoxy; 4-(methylcarbonylamino)phenoxy; 4-(methylsulfonamido)phenoxy; 4-fluorophenoxy; 4-methoxyphenoxy; 4-methoxyphenylsulfanyl; 4-methylphenyl; bromo; chloro; dimethylaminomethyl; ethoxy; ethylsulfanyl; hydrogen; isopropyl; methoxy; methoxymethyl; methyl; N,N-dimethylaminocarbonyl; naphth-2-yloxy; naphthylsulfanyl; phenoxy; phenyl; phenylamino; phenylsulfinyl; phenylsulfanyl; pyridin-2-yloxy; pyridin-2-yl; and pyridin-2-ylsulfanyl.

In compounds of formulae (IV), (IVA), (IVB), (IVC), and (IVD), R² is preferably selected from the group consisting of substituted amino, aryloxy, substituted aryloxy, alkoxy, substituted alkoxy, halo, hydrogen, alkyl, substituted alkyl, aryl, —S(O)ₙ-aryl, —S(O)ₙ-substituted aryl, —S(O)ₙ-cycloalkyl, where n is zero, one 20 or two, aminocarbonylamino, heteroaryloxy, and cycloalkyloxy.

More preferably, R² is selected from the group consisting of (4-methoxy)phenylsulfonylamino; 2,6-dimethylphenoxy; 3,4-difluorophenoxy; 3,5-difluorophenoxy; 3-chloro-4-fluorophenoxy; 3-methoxy-4-fluorophenoxy; 3-methoxy-5-fluorophenoxy; 4-(methylsulfonamido)phenoxy; 4-(phenylsulfonamido)phenoxy; 4-CF₃-O-phenoxy; 4-CF₃-phenoxy; 4-chlorophenoxy; 4-fluorophenoxy; 4-(4-fluorophenoxy)phenoxy; 4-methoxyphenoxy; 4-nitrophenoxy; benzyloxy; bromo; butoxy; CF₃; chloro; cyclohexyloxy; cyclohexylsulfanyl; cyclohexylsulfonyl; fluoro; hydrogen; iodo; isopropoxy; methyl; phenoxy; phenyl; phenylsulfanyl; phenylsulfinyl; phenylsulfonyl; phenylurea; pyridin-1-ylsulfanyl; pyridin-3-yloxy; and pyridin4-ylsulfanyl.

In compounds of formulae (WV), (IVA), (IVB), (IVC), and (IVD), R³ is preferably selected from the group consisting of: substituted aryloxy, substituted alkoxy, alkoxy, substituted alkyl, alkyl, amino, cycloalkyloxy, hydrogen, halo, aryl, —S(O)ₙ-aryl, —S(O)ₙ-substituted aryl, —S(O)ₙ-heteroaryl, and —S(O)ₙ-substituted heteroaryl, where n is zero, one or two, aminocarbonylamino, and heteroaryloxy.

More preferably, R³ is selected from the group consisting of amino; (4-methyl)phenyl-sulfonylaminophenoxy; 3,4-difluorophenoxy; 3,5-difluorophenoxy; 3-fluoro-5-methoxy-phenoxy; 3-Chloro-4-fluorophenoxy 4-CF₃—O-phenoxy; 4-CF₃-phenoxy; 4-chlorophenoxy; 4-fluorophenoxy; 4-(4-fluorophenoxy)phenoxy; 4-methoxyphenoxy; benzyloxy; bromo; butoxy; CF₃; chloro; cyclohexyloxy; hydrogen; iodo; isopropoxy; phenoxy; phenyl; phenylsulfanyl; phenylsulfonyl; phenylsulfinyl; phenylurea; pyridin-1-ylsulfanyl; pyridin-3-yloxy; and pyridin-4-ylsulfanyl.

Alternatively, R² and R³, combined with the carbon atoms pendent thereto, are joined to form an aryl group. Preferably, the aryl group is phenyl.

In compounds of formulae (IV), (IVA), (IVB), (IVC), and (IVD), R⁴ is preferably selected from the group consisting of: substituted arylthio, halo, hydrogen, substituted alkyl and aryl.

More preferably, R⁴ is selected from the group consisting of 4-chlorophenyl sulfanyl; chloro; hydrogen; methoxymethyl; and phenyl.

In compounds of formulae (IV), (IVA), (IVB), (IVC), and (IVD), R⁵ is preferably hydrogen or aryl. More preferably R⁵ is hydrogen or phenyl.

In compounds of formulae (IV), (IVA) and (IVC), R is preferably selected from the group consisting of hydrogen, deuterium, aryl and alkyl. More preferably R is selected from the group consisting of phenyl, hydrogen, deuterium and methyl.

In compounds of formulae (IV), (IVA) and (IVC), R⁴⁰ is selected from the group consisting of preferably hydrogen, deuterium, alkyl, substituted alkyl, and substituted amino. More preferably, R⁴⁰ is selected from the group consisting of 4-aminobutyl; 4-hydroxybenzyl; benzyl; carboxylmethyl; deuterium; hydroxymethyl; imidazol-4-ylmethyl; isopropyl; methyl; and propyl.

Alternatively, R, R′ and the carbon atom pendent thereto join to form a cycloalkyl and more preferably cyclopropyl.

In compounds of formulae (IV), (IVA) and (IVC), R″ is preferably hydrogen, alkyl or substituted alkyl. More preferably, R″ is hydrogen, methyl or carboxylmethyl (—CH₂C(O)OH). Alternatively, R′, R″ and the carbon atom and nitrogen atom respectively pendent thereto join to form a heterocyclic group and more preferably pyrrolidinyl.

In compounds of formulae (IV), (IVA), (IVB), (IVC) and (IVD), preferably R′″ is selected from the group consisting of hydrogen, hydroxy, alkoxy, substituted alkoxy, cycloalkoxy, substituted cycloalkoxy, thiol, acyloxy and aryl. Preferably, R′″ is selected from the group consisting of hydroxy; benzyloxy; ethoxy; thiol; methoxy; methylcarbonyloxy; and phenyl.

In compounds of formulae (IV), (IVB) and (IVC), WR⁸ is preferably selected from the group consisting of amino, substituted amino, aminoacyl, hydroxy, and alkoxy. More preferably, WR⁸ is selected from the group consisting of amino; dimethylamino; hydroxy; methoxy; and methylcarbonylamino.

Representative compounds of formulae (WV), (IVA), (IVB), (IVC) and (IVD) are presented in Tables A-D, wherein said table letter corresponds to formula letter (i.e., representative compounds of formula IVA re in Table A).

TABLE A

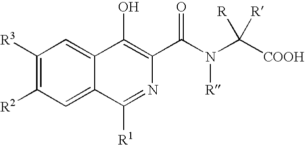

| No. | R¹ | R² | R³ | R | R′ | R″ |
|---|---|---|---|---|---|---|
| 1 | Cl | H | benzyloxy | H | methyl | H |
| 2 | Cl | H | H | H | hydroxymethyl | H |
| 3 | Cl | H | H | H | hydroxymethyl | H |
| 4 | Cl | H | isopropoxy | H | hydroxymethyl | H |
| 5 | Cl | H | isopropoxy | H | hydroxymethyl | H |
| 6 | Cl | isopropoxy | H | H | hydroxymethyl | H |
| 7 | Cl | isopropoxy | H | H | hydroxymethyl | H |
| 8 | Cl | H | H | methyl | methyl | H |
| 9 | Cl | H | isopropoxy | methyl | methyl | H |
| 10 | Cl | H | H | H | imidazol-4-ylmethyl | H |
| 11 | Cl | H | H | H | imidazol-4-ylmethyl | H |
| 12 | Cl | H | H | H | isopropyl | H |
| 13 | Cl | H | H | H | isopropyl | H |
| 14 | Cl | H | isopropoxy | H | isopropyl | H |
| 15 | Cl | H | isopropoxy | H | isopropyl | H |
| 16 | Cl | isopropoxy | H | H | isopropyl | H |
| 17 | Cl | isopropoxy | H | H | isopropyl | H |
| 18 | Cl | H | benzyloxy | H | isopropyl | H |
| 19 | Cl | H | H | H | benzyl | H |
| 20 | Cl | H | H | H | benzyl | H |
| 21 | Cl | H | isopropoxy | H | benzyl | H |
| 22 | Cl | H | isopropoxy | H | benzyl | H |
| 23 | Cl | isopropoxy | H | H | benzyl | H |
| 24 | Cl | isopropoxy | H | H | benzyl | H |
| 25 | Cl | H | H | H | 4-hydroxybenzyl | H |
| 26 | Cl | H | H | H | 4-hydroxybenzyl | H |
| 27 | Cl | H | isopropoxy | H | 4-hydroxybenzyl | H |
| 28 | Cl | H | isopropoxy | H | 4-hydroxybenzyl | H |
| 29 | Cl | isopropoxy | H | H | 4-hydroxybenzyl | H |
| 30 | Cl | isopropoxy | H | H | 4-hydroxybenzyl | H |
| 31 | Cl | H | isopropoxy | H | propyl | H |
| 32 | Cl | H | isopropoxy | H | propyl | H |
| 33 | Cl | H | H | H | R′ and R″ and the carbon | — |
| | | | | | and nitrogen atom | |
| | | | | | respectively pendent to | |
| | | | | | which R″ is attached join to | |
| | | | | | form a pyrrolidinyl | |
| 34 | Cl | H | H | H | R′ and R″ and the carbon | — |
| | | | | | and nitrogen atom | |
| | | | | | respectively pendent to | |
| | | | | | which R″ is attached join to | |
| | | | | | form a pyrrolidinyl | |
| 35 | Cl | H | isopropoxy | H | R′ and R″ and the carbon | — |
| | | | | | and nitrogen atom | |
| | | | | | respectively pendent to | |
| | | | | | which R″ is attached join to | |
| | | | | | form a pyrrolidinyl | |
| 36 | Cl | H | isopropoxy | H | R′ and R″ and the carbon | — |
| | | | | | and nitrogen atom | |
| | | | | | respectively pendent to | |
| | | | | | which R″ is attached join to | |
| | | | | | form a pyrrolidinyl | |
| 37 | Cl | H | H | H | 4-aminobutyl | H |
| 38 | Cl | H | H | H | 4-aminobutyl | H |
| 39 | Cl | H | isopropoxy | H | 4-aminobutyl | H |
| 40 | CI | H | isopropoxy | H | 4-aminobutyl | H |
| 41 | Cl | isopropoxy | H | H | 4-aminobutyl | H |
| 42 | Cl | isopropoxy | H | H | 4-aminobutyl | H |
| 43 | Cl | H | H | H | carboxylmethyl | H |
| 44 | Cl | H | H | H | carboxylmethyl | H |
| 45 | Cl | H | isopropoxy | H | carboxylmethyl | H |
| 46 | Cl | H | isopropoxy | H | carboxylmethyl | H |
| 47 | Cl | isopropoxy | H | H | carboxylmethyl | H |
| 48 | Cl | H | H | — | R, R′ together with the | H |
| | | | | | carbon to which they are | |
| | | | | | attached join to form | |
| | | | | | cyclopropyl | |
| 49 | Cl | H | isopropoxy | — | R, R′ together with the | H |
| | | | | | carbon to which they are | |
| | | | | | attached join to form | |
| | | | | | cyclopropyl | |
| 50 | Cl | H | H | D | D | H |
| 51 | Cl | H | benzyloxy | H | methyl | H |
| 52 | Cl | benzyloxy | H | H | methyl | H |
| 53 | Cl | benzyloxy | H | H | methyl | H |
| 54 | Cl | H | H | H | methyl | H |
| 55 | Cl | H | H | H | methyl | H |
| 56 | Cl | H | isopropoxy | H | methyl | H |
| 57 | Cl | H | isopropoxy | H | methyl | H |
| 58 | Cl | isopropoxy | H | H | methyl | H |
| 59 | Cl | isopropoxy | H | H | methyl | H |
| 60 | H | 4-chlorophenoxy | H | H | methyl | H |
| 61 | H | H | 4-chlorophenoxy | H | methyl | H |
| 62 | H | 3,4-difluorophenoxy | H | H | methyl | H |
| 63 | H | phenylsulfanyl | H | H | methyl | H |
| 64 | H | phenylsulfanyl | H | H | methyl | H |
| 65 | H | phenoxy | H | H | methyl | H |
| 66 | H | 4-methoxyphenoxy | H | H | methyl | H |
| 67 | H | phenylsulfonyl | H | H | methyl | H |
| 68 | methoxy | phenoxy | H | H | methyl | H |
| | methyl | | | | | |
| 69 | methoxy | phenoxy | H | H | methyl | H |
| | methyl | | | | | |
| 70 | H | phenoxy | H | H | methyl | H |
| 71 | 4- | H | H | H | methyl | H |
| | chloro- | | | | | |
| | phenyl | | | | | |
| | sulfanyl | | | | | |
| 72 | 4- | H | H | H | methyl | H |
| | chloro- | | | | | |
| | phenyl | | | | | |
| | sulfanyl | | | | | |
| 73 | H | 3-methoxy-4- | H | H | methyl | H |
| | | fluorophenoxy | | | | |
| 74 | H | cyclohexyloxy | H | H | methyl | H |
| 75 | methyl | 4-fluorophenoxy | H | H | methyl | H |
| 76 | H | 4-fluorophenoxy | H | H | methyl | H |
| 77 | methyl | phenoxy | H | H | methyl | H |
| 78 | methyl | phenylsulfanyl | H | H | methyl | H |
| 79 | H | 4-trifluoromethyl- | H | H | methyl | H |
| | | phenoxy | | | | |

TABLE B

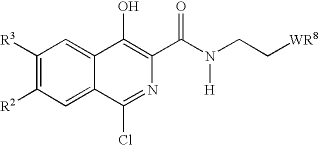

| No. | R² | R³ | WR⁸ |
|---|---|---|---|
| 1 | H | H | methoxy |
| 2 | isopropoxy | H | amino |
| 3 | H | isopropoxy | methoxy |
| 4 | H | H | amino |
| 5 | H | H | hydroxy |
| 6 | H | isopropoxy | hydroxy |
| 7 | H | H | dimethylamino |
| 8 | H | H | methylcarbonylamino |
| 9 | H | isopropoxy | amino |
| 10 | H | isopropoxy | dimethylamino |
| 11 | isopropoxy | H | methoxy |
| 12 | isopropoxy | H | dimethyl amino |
| 13 | isopropoxy | H | hydroxy |

TABLE C

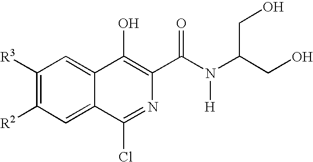

| No. | R² | R³ |
|---|---|---|
| 1 | isopropoxy | H |
| 2 | H | isopropoxy |
| 3 | H | H |

TABLE D

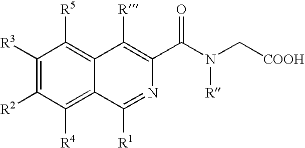

| No. | R¹ | R² | R³ | R⁴ | R⁵ | R″ | R′″ |
|---|---|---|---|---|---|---|---|
| 1 | Br | 2,6- | H | H | H | H | OH |
| | | di(CH₃)phenyloxy | | | | | |
| 2 | Br | butoxy | H | H | H | H | OH |
| 3 | Br | phenoxy | H | H | H | H | OH |
| 4 | Cl | Br | H | H | H | H | OH |
| 5 | Br | Cl | H | H | H | H | OH |
| 6 | Cl | I | H | H | H | H | OH |
| 7 | Cl | H | I | H | H | H | OH |
| 8 | Cl | phenoxy | H | H | H | H | OH |
| 9 | Cl | Phenylsulfanyl | H | H | H | H | OH |
| 10 | Br | —CF₃ | H | H | H | H | OH |
| 11 | Br | H | phenoxy | H | H | H | OH |
| 12 | Cl | H | H | phenyl H | H | OH | |
| 13 | Cl | 2,6- | H | H | H | H | OH |
| | | di(CH₃)phenyloxy | | | | | |
| 14 | Br | H | CF₃ | H | H | H | OH |
| 15 | Br | Br | H | H | H | H | OH |
| 16 | Br | phenylsulfanyl | H | H | H | H | OH |
| 17 | Cl | H | phenylsulfanyl | H | H | H | OH |
| 18 | 4-methoxy phenyl- | H | H | H | H | H | OH |
| | sulfanyl | | | | | | |
| 19 | Br | H | H | phenyl | H | H | OH |
| 20 | Cl | phenyl | H | H | H | H | OH |
| 21 | Br | H | H | H | H | H | OH |
| 22 | Br | methyl | H | H | H | H | OH |
| 23 | Br | H | butoxy | H | H | H | OH |
| 24 | Br | H | Cl | H | H | H | OH |
| 25 | Cl | H | phenoxy | H | H | H | OH |
| 26 | Br | H | phenoxy | H | H | H | OH |
| 27 | H | I | H | H | H | H | OH |
| 28 | Br | phenyl | H | H | H | H | OH |
| 29 | Br | H | phenyl | H | H | H | OH |
| 30 | ethyl sulfanyl | H | H | H | H | H | OH |
| 31 | phenoxy | H | H | H | H | H | OH |
| 32 | H | H | phenyl | H | H | H | OH |
| 33 | Br | H | H | H | phenyl | H | OH |
| 34 Br | F | H | H | H | H | OH | |
| 35 | H | 2,6-di(CH₃) | H | H | H | H | OH |
| | | phenyloxy | | | | | |
| 36 | Cl | H | phenyl | H | H | H | OH |
| 37 | H | phenoxy | H | H | H | H | OH |
| 38 | H | phenylsulfanyl | H | H | H | H | OH |
| 39 | H | phenyl | H | H | H | H | OH |
| 40 | H | H | phenoxy | H | H | H | OH |
| 41 | H | H | phenylsulfanyl | H | H | H | OH |
| 42 | H | H | H | phenyl | H | H | OH |
| 43 | Cl | H | H | H | phenyl | H | OH |
| 44 | H | H | H | H | phenyl | H | OH |
| 45 CI | F | H | H | H | H | OH | |
| 46 | H | F | H | H | H | H | OH |
| 47 | H | H | Br | H | H | H | OH |
| 48 | H | R²/R³ = phenyl | — | H | H | H | OH |
| 49 | Br | H | benzyloxy | H | H | methyl | OH |
| 50 | CI | H | H | H | H | methyl | OH |
| 51 | CI | H | isopropoxy | H | H | methyl | OH |
| 52 | CI | isopropoxy | H | H | H | methyl | OH |
| 53 | Cl | H | H | H | H | CH₂COOH | OH |
| 54 | Cl | H | isopropoxy | H | H | CH₂COOH | OH |
| 55 | naphth-2-yloxy | H | H | H | H | H | OH |
| 56 | pyridin-3-yloxy | H | H | H | H | H | OH |
| 57 | 4-methoxy phenoxy | H | H | H | H | H | OH |
| 58 | 3-methoxy phenoxy | H | H | H | H | H | OH |
| 59 | 3-fluorophenoxy | H | H | H | H | H | OH |
| 60 | 4-fluorophenoxy | H | H | H | H | H | OH |
| 61 | 2-fluorophenoxy | H | H | H | H | H | OH |
| 62 | 2-methoxy phenoxy | H | H | H | H | H | OH |
| 63 | 4-(methyl carbonyl | H | H | H | H | H | OH |
| | amino) phenoxy | | | | | | |
| 64 | 4-(methyl | H | H | H | H | H | OH |
| | sulfonamido) | | | | | | |
| | phenoxy | | | | | | |
| 65 | phenyl amino | H | H | H | H | H | OH |
| 66 | H | H | pyridin-3-yloxy H | H | H | OH | |
| 67 | H | pyridin-3-yloxy | H | H | H | H | OH |
| 68 | Cl | H | H | H | H | H | methoxy |
| 69 | Cl | H | H | H | H | H | ethoxy |
| 70 | methoxy | H | H | H | H | H | OH |
| 71 | ethoxy | H | H | H | H | H | OH |
| 72 | phenyl | H | H | H | H | H | methyl- |
| | | | | | | | carbonyloxy |
| 73 | phenyl | H | H | H | H | H | OH |
| 74 | ethoxy | H | H | H | H | H | phenyl |
| 75 | Cl | H | H | H | H | H | phenyl |
| 76 | H | H | H | H | H | H | phenyl |
| 77 | methyl | H | H | H | H | H | OH |
| 78 | methoxy methyl | H | H | H | H | H | OH |
| 79 | N,N-dimethyl | H | H | H | H | H | OH |
| | amino carbonyl | | | | | | |
| 80 | methyl | H | phenoxy | H | H | H | OH |
| 81 | methyl | phenoxy | H | H | H | H | OH |
| 82 | methyl | phenoxy | H | H | H | H | benzyloxy |
| 83 | methyl | phenoxy | H | H | H | H | ethoxy |
| 84 | N,N-dimethyl | phenoxy | H | H | H | H | OH |
| | amino carbonyl | | | | | | |
| 85 | methoxy methyl | phenoxy | H | H | H | H | OH |
| 86 | 4-methyl phenyl | H | H | H | H | H | OH |
| 87 | methyl | 4-fluoro phenoxy | H | H | H | H | OH |
| 88 | Cl | 4-methoxy | H | H | H | H | OH |
| | | phenoxy | | | | | |
| 89 | H | 4-methoxy | H | H | H | H | OH |
| | | phenoxy | | | | | |
| 90 | Cl | H | 4-methoxy- | H | H | H | OH |
| | | | phenoxy | | | | |
| 91 | H | H | 4-methoxy- | H | H | H | OH |
| | | | phenoxy | | | | |
| 92 | Cl | 4-CF₃-phenoxy | H | H | H | H | OH |
| 93 | H | 4-CF₃-phenoxy | H | H | H | H | OH |
| 94 | Cl | H | 4-CF₃-phenoxy | H | H | H | OH |
| 95 | H | H | 4-CF₃-phenoxy | H | H | H | OH |
| 96 | Cl | 4-fluorophenoxy | H | H | H | H | OH |
| 97 | H | 4-fluorophenoxy | H | H | H | H | OH |
| 98 | Cl | H | 4-fluoro- | H | H | H | OH |
| | | | phenoxy | | | | |
| 99 | H | H | 4-fluoro- | H | H | H | OH |
| | | | phenoxy | | | | |
| 100 | H | pyridin-4-yl | H | H | H | H | OH |
| | | sulfanyl | | | | | |
| 101 | H | H | pyridin-4-yl | H | H | H | OH |
| | | | sulfanyl | | | | |
| 102 | H | phenylsulfinyl | H | H | H | H | OH |
| 103 | H | phenylsulfonyl | H | H | H | H | OH |
| 104 | H | H | phenyl sulfinyl H | H | H | OH | |
| 105 | H | H | phenyl sulfonyl H | H | H | OH | |
| 106 | H | H | amino | H | H | H | OH |
| 107 | H | (4-methoxy) | H | H | H | H | OH |
| | | phenylsulfonyl | | | | | |
| | | amino | | | | | |
| 108 | H | phenylurea | H | H | H | H | OH |
| 109 | H | H | phenylurea | H | H | H | OH |
| 110 | phenyl sulfanyl | H | H | H | H | H | OH |
| 111 | (4-chloro phenyl) | H | H | H | H | H | OH |
| | sulfanyl | | | | | | |
| 112 | (4-methyl phenyl) | H | H | H | H | H | OH |
| | sulfanyl | | | | | | |
| 113 | pyridin-2-ylsulfanyl | H | H | H | H | H | OH |
| 114 | (3-methoxy phenyl) | H | H | H | H | H | OH |
| | sulfanyl | | | | | | |
| 115 | 2-methoxy phenyl | H | H | H | H | H | OH |
| | sulfanyl | | | | | | |
| 116 | naphthyl sulfanyl | H | H | H | H | H | OH |
| 117 | phenylsulfinyl | H | H | H | H | H | OH |
| 118 | phenylsulfonyl | H | H | H | H | H | OH |
| 119 | H | pyridin-2-yl | H | H | H | H | OH |
| | | sulfanyl | | | | | |
| 120 | H | H | pyridin-2-yl | H | H | H | OH |
| | | | sulfanyl | | | | |
| 121 | Cl | phenoxy | phenoxy | H | H | H | OH |
| 122 | H | phenoxy | phenoxy | H | H | H | OH |
| 123 | H | H | (4- | H | H | H | OH |
| | | methyl)phenyl | | | | | |
| | | SO₂—NH- | | | | | |
| | | phenoxy | | | | | |
| 124 | H | 4-nitrophenoxy | H | H | H | H | OH |
| 125 | H | phenoxy | H | H | H | H | thiol |
| 126 | H | CF₃ | H | H | H | H | thiol |
| 127 | H | 4-(phenyl- | H | H | H | H | OH |
| | | sulfonamido) | | | | | |
| | | phenoxy | | | | | |
| 128 | H | 4-(methyl- | H | H | H | H | OH |
| | | sulfonamido) phenoxy | | | | | |
| 129 | H | 4-chlorophenoxy | H | H | H | H | OH |
| 130 | H | H | 4-chloro- | H | H | H | OH |
| | | | phenoxy | | | | |
| 131 | H | H | 3-fluoro-5- | H | H | H | OH |
| | | | methoxy- | | | | |
| | | | phenoxy | | | | |
| 132 | H | 3-methoxy-5- | H | H | H | H | OH |
| | | fluorophenoxy | | | | | |
| 133 | H | 3,4- | H | H | H | H | OH |
| | | difluorophenoxy | | | | | |
| 134 | H | H | 3,4-difluoro- | H | H | H | OH |
| | | | phenoxy | | | | |
| 135 | H | 4-CF₃—O-phenoxy | H | H | H | H | OH |
| 136 | H | H | 4-CF₃—O- | H | H | H | OH |
| | | | phenoxy | | | | |
| 137 | H | 3,5- | H | H | H | H | OH |
| | | difluorophenoxy | | | | | |
| 138 | H | H | 3,5- | H | H | H | OH |
| | | | difluorophenoxy | | | | |
| 139 | H | 4-(4- | H | H | H | H | OH |
| | | fluorophenoxy) | | | | | |
| | | phenoxy | | | | | |
| 140 | H | H | 4-(4- | H | H | H | OH |
| | | | fluorophenoxy) | | | | |
| | | | phenoxy | | | | |
| 141 | H | 3-chloro-4- | H | H | H | H | OH |
| | | fluorophenoxy | | | | | |
| 142 | H | H | 3-chloro-4- | H | H | H | OH |
| | | | fluorophenoxy | | | | |
| 143 | methyl | 4-chlorophenoxy | H | H | H | H | OH |
| 144 | methyl | H | 4- | H | H | H | OH |
| | | | chlorophenoxy | | | | |
| 145 | methyl | 3,5- | H | H | H | H | OH |
| | | difluorophenoxy | | | | | |
| 146 | methyl | 4-methoxy | H | H | H | H | OH |
| | | phenoxy | | | | | |
| 147 | methyl | H | 4- | H | H | H | OH |
| | | | methoxyphenoxy | | | | |
| 148 | H | H | cyclohexyloxy | H | H | H | OH |
| 149 | H | cyclohexyloxy | H | H | H | H | OH |
| 150 | methyl | cyclohexyloxy | H | H | H | H | OH |
| 151 | H | cyclohexyl | H | H | H | H | OH |
| | | sulfanyl | | | | | |
| 152 | H | cyclohexyl | H | H | H | H | OH |
| | | sulfonyl | | | | | |
| 153 | isopropyl | H | H | H | H | H | OH |
| 154 | pyridin-2-yl | H | H | H | H | H | OH |
| 155 | ethyl | phenoxy | H | H | H | H | OH |
| 156 | dimethyl amino | phenylsulfanyl | H | H | H | H | OH |
| | methyl | | | | | | |
| 157 | methyl | phenylsulfanyl | H | H | H | H | OH |
| 158 | methyl | 4-trifluoromethyl | H | H | H | H | OH |
| | | phenoxy | | | | | |

Compounds included within the scope of this invention include, for example, those set forth below: {[4-Hydroxy-1-(naphthalen-2-yloxy)-isoquinoline-3-carbonyl]-amino}-acetic acid; {[4-Hydroxy-1-(pyridin-3-yloxy)-isoquinoline-3-carbonyl]-amino}-acetic acid; {[4-Hydroxy-1-(4-methoxy-phenoxy)-isoquinoline-3-carbonyl]-amino}-acetic acid; {[4-Hydroxy-1-(3-methoxy-phenoxy)-isoquinoline-3-carbonyl]-amino}-acetic acid; {[1-(3-Fluoro-phenoxy)-4-hydroxy-isoquinoline-3-carbonyl]-amino}-acetic acid; {[1-(4-Fluoro-phenoxy)-4-hydroxy-isoquinoline-3-carbonyl]-amino}-acetic acid; {[1-(2-Fluoro-phenoxy)-4-hydroxy-isoquinoline-3-carbonyl]-amino}-acetic acid; {[4-Hydroxy-1-(2-methoxy-phenoxy)-isoquinoline-3-carbonyl]-amino}-acetic acid; {[1-(4-Acetylamino-phenoxy)-4-hydroxy-isoquinoline-3-carbonyl]-amino}-acetic acid; {[4-Hydroxy-1-(4-methanesulfonylamino-phenoxy)-isoquinoline-3-carbonyl]-amino}-acetic acid; [(4-Hydroxy-1-phenylamino-isoquinoline-3-carbonyl)-amino]-acetic acid; {[4-Hydroxy-6-(pyridin-3-yloxy)-isoquinoline-3-carbonyl]-amino}-acetic acid; {[4-Hydroxy-7-(pyridin-3-yloxy)-isoquinoline-3-carbonyl]-amino}-acetic acid; [(1-Chloro-4-methoxy-isoquinoline-3-carbonyl)-amino]-acetic acid; [(1-Chloro-4-ethoxy-isoquinoline-3-carbonyl)-amino]-acetic acid; [(4-Hydroxy-1-methoxy-isoquinoline-3-carbonyl)-amino]-acetic acid; [(1-Ethoxy-4-hydroxy-isoquinoline-3-carbonyl)-amino]-acetic acid; [(4-Acetoxy-1-phenyl-isoquinoline-3-carbonyl)-amino]-acetic acid; [(4-Hydroxy-1-phenyl-isoquinoline-3-carbonyl)-amino]-acetic acid; [(1-Ethoxy-4-phenyl-isoquinoline-3-carbonyl)-amino]-acetic acid; [(1-Chloro-4-phenyl-isoquinoline-3-carbonyl)-amino]-acetic acid; [(4-Phenyl-isoquinoline-3-carbonyl)-amino]-acetic acid; [(4-Hydroxy-1-methyl-isoquinoline-3-carbonyl)-amino]-acetic acid; [(4-Hydroxy-1-methoxymethyl-isoquinoline-3-carbonyl)-amino]-acetic acid; [(1-Dimethylcarbamoyl-4-hydroxy-isoquinoline-3-carbonyl)-amino]-acetic acid; [(4-Hydroxy-1-methyl-6-phenoxy-isoquinoline-3-carbonyl)-amino]-acetic acid; [(4-Hydroxy-1-methyl-7-phenoxy-isoquinoline-3-carbonyl)-amino]-acetic acid; [(4-Benzyloxy-1-methyl-7-phenoxy-isoquinoline-3-carbonyl)-amino]-acetic acid; [(4-Ethoxy-1-methyl-7-phenoxy-isoquinoline-3-carbonyl)-amino]-acetic acid; [(1-Dimethylcarbamoyl-4-hydroxy-7-phenoxy-isoquinoline-3-carbonyl)-amino]-acetic acid; [(4-Hydroxy-1-methoxymethyl-7-phenoxy-isoquinoline-3-carbonyl)-amino]-acetic acid; [(4-Hydroxy-1-p-tolyl-isoquinoline-3-carbonyl)-amino]-acetic acid; {[7-(4-Fluoro-phenoxy)-4-hydroxy-1-methyl-isoquinoline-3-carbonyl]-amino}-acetic acid; {[1-Chloro-4-hydroxy-7-(4-methoxy-phenoxy)-isoquinoline-3-carbonyl]-amino}-acetic acid; {[4-Hydroxy-7-(4-methoxy-phenoxy)-isoquinoline-3-carbonyl]-amino}-acetic acid; {[1-Chloro-4-hydroxy-6-(4-methoxy-phenoxy)-isoquinoline-3-carbonyl]-amino}-acetic acid; {[4-Hydroxy-6-(4-methoxy-phenoxy)-isoquinoline-3-carbonyl]-amino}-acetic acid; {[1-Chloro-4-hydroxy-7-(4-trifluoromethyl-phenoxy)-isoquinoline-3-carbonyl]-amino}-acetic acid; {[4-Hydroxy-7-(4-trifluoromethyl-phenoxy)-isoquinoline-3-carbonyl]-amino}-acetic acid; {[1-Chloro-4-hydroxy-6-(4-trifluoromethyl-phenoxy)-isoquinoline-3-carbonyl]-amino) -acetic acid; {[4-Hydroxy-6-(4-trifluoromethyl-phenoxy)-isoquinoline-3-carbonyl]-amino}-acetic acid; {[1-Chloro-7-(4-fluoro-phenoxy)-4-hydroxy-isoquinoline-3-carbonyl]-amino) -acetic acid; {[7-(4-Fluoro-phenoxy)-4-hydroxy-isoquinoline-3-carbonyl]-amino}-acetic acid; {[1-Chloro-6-(4-fluoro-phenoxy)-4-hydroxy-isoquinoline-3-carbonyl]-amino}-acetic acid; {[6-(4-Fluoro-phenoxy)-4-hydroxy-isoquinoline-3-carbonyl]-amino}-acetic acid; {[4-Hydroxy-7-(pyridin4-ylsulfanyl)-isoquinoline-3-carbonyl]-amino) -acetic acid; {[4-Hydroxy-6-(pyridin-4-ylsulfanyl)-isoquinoline-3-carbonyl]-amino}-acetic acid; [(7-Benzenesulfinyl-4-hydroxy-isoquinoline-3-carbonyl)-amino]-acetic acid; [(7-Benzenesulfonyl-4-hydroxy-isoquinoline-3-carbonyl)-amino]-acetic acid; [(6-Benzenesulfinyl-4-hydroxy-isoquinoline-3-carbonyl)-amino]-acetic acid; [(6-Benzenesulfonyl-4-hydroxy-isoquinoline-3-carbonyl)-amino]-acetic acid; [(6-Amino-4-hydroxy-isoquinoline-3-carbonyl)-amino]-acetic acid; {[4-Hydroxy-7-(4-methoxy-benzenesulfonylamino)-isoquinoline-3-carbonyl]-amino}-acetic acid; {[4-Hydroxy-7-(3-phenyl-ureido)-isoquinoline-3-carbonyl]-amino) -acetic acid; {[4-Hydroxy-6-(3-phenyl-ureido)-isoquinoline-3-carbonyl]-amino}-acetic acid; [(4-Hydroxy-1-phenylsulfanyl-isoquinoline-3-carbonyl)-amino]-acetic acid; {[1-(4-Chloro-phenylsulfanyl)-4-Hydroxy-isoquinoline-3-carbonyl]-amino}-acetic acid; [(4-Hydroxy-1-p-tolylsulfanyl-isoquinoline-3-carbonyl)-amino]-acetic acid; {[4-Hydroxy-1-(pyridin-2-ylsulfanyl)-isoquinoline-3-carbonyl]-amino}-acetic acid; {[4-Hydroxy-1-(3-methoxy-phenylsulfanyl)-isoquinoline-3-carbonyl]-amino}-acetic acid; {[4-Hydroxy-1-(2-methoxy-phenylsulfanyl)-isoquinoline-3-carbonyl]-amino}-acetic acid; {[4-Hydroxy-1-(naphthalen-2-ylsulfanyl)-isoquinoline-3-carbonyl]-amino) -acetic acid; [(1-Benzenesulfinyl-4-hydroxy-isoquinoline-3-carbonyl)-amino]-acetic acid; [(1-Benzenesulfonyl-4-hydroxy-isoquinoline-3-carbonyl)-amino]-acetic acid; {[4-Hydroxy-7-(pyridin-2-ylsulfanyl)-isoquinoline-3-carbonyl]-amino) -acetic acid; {[4-Hydroxy-6-(pyridin-2-ylsulfanyl)-isoquinoline-3-carbonyl]-amino}-acetic acid; [(1-Chloro-4-hydroxy-6,7-diphenoxy-isoquinoline-3-carbonyl)-amino]-acetic acid; [(4-Hydroxy-6,7-diphenoxy-isoquinoline-3-carbonyl)-amino]-acetic acid; ({4-Hydroxy-7-[4-(toluene4-sulfonylamino)-phenoxy]-isoquinoline-3-carbonyl}-amino)-acetic acid; {[4-Hydroxy-7-(4-nitro-phenoxy)-isoquinoline-3-carbonyl]-amino}-acetic acid; [(4-Mercapto-7-phenoxy-isoquinoline-3-carbonyl)-amino]-acetic acid; [(4-Mercapto-7-trifluoromethyl-isoquinoline-3-carbonyl)-amino]-acetic acid; {[7-(4-Benzenesulfonylamino-phenoxy)-4-hydroxy-isoquinoline-3-carbonyl]-amino) -acetic acid; {[4-Hydroxy-7-(4-methanesulfonylamino-phenoxy)-isoquinoline-3-carbonyl]-amino}-acetic acid; {[7-(4-Chloro-phenoxy)-4-hydroxy-isoquinoline-3-carbonyl]-amino}-acetic acid; {[6-(4-Chloro-phenoxy)-4-hydroxy-isoquinoline-3-carbonyl]-amino}-acetic acid; {[6-(3-Fluoro-5-methoxy-phenoxy)-4-hydroxy-isoquinoline-3-carbonyl]-amino}-acetic acid; {[7-(3-Fluoro-5-methoxy-phenoxy)-4-hydroxy-isoquinoline-3-carbonyl]-amino -acetic acid; [7-(3,4-Difluoro-phenoxy)4-hydroxy-isoquinoline-3-carbonyl]-amino}-acetic acid; {[6-(3,4-Difluoro-phenoxy)-4-hydroxy-isoquinoline-3-carbonyl]-amino) -acetic acid; {[4-Hydroxy-7-(4-trifluoromethoxy-phenoxy)-isoquinoline-3-carbonyl]-amino}-acetic acid; {[4-Hydroxy-6-(4-trifluoromethoxy-phenoxy)-isoquinoline-3-carbonyl]-amino}-acetic acid; 2-(S)-{[7-(4-Chloro-phenoxy)-4-hydroxy-isoquinoline-3-carbonyl]- amino}-propionic acid; 2-(S)-{[6-(4-Chloro-phenoxy)-4-hydroxy-isoquinoline-3-carbonyl]-amino}-propionic acid; 2-{[7-(3,4-Difluoro-phenoxy)-4-hydroxy-isoquinoline-3-carbonyl]-amino}-propionic acid; 2-(S)-[(4-Hydroxy-7-phenylsulfanyl-isoquinoline-3-carbonyl)-amino]-propionic acid.; 2-(R)-[(4-Hydroxy-7-phenylsulfanyl-isoquinoline-3-carbonyl)-amino]-propionic acid; 2-(R)-[(4-Hydroxy-7-phenoxy-isoquinoline-3-carbonyl)-amino]-propionic acid; 2-(S)-{[4-Hydroxy-7-(4-methoxy-phenoxy)-isoquinoline-3-carbonyl]-amino}-propionic acid; 2-(S)-[(7-Benzenesulfonyl-4-hydroxy-isoquinoline-3-carbonyl)-amino]-propionic acid; (R)-2-[(4-Hydroxy-1-methoxymethyl-7-phenoxy-isoquinoline-3-carbonyl)-amino]-propionic acid; (S)-2-[(4-Hydroxy-1-methoxymethyl-7-phenoxy-isoquinoline-3-carbonyl)-amino]-propionic acid; (S)-2-[(4-Mercapto-7-phenoxy-isoquinoline-3-carbonyl)-amino]-propionic acid; (S)-2-({[1-(4-Chloro-phenylsulfanyl)-4-hydroxy-isoquinoline-3-carbonyl]-amino}-propionic acid; (R)-2-{[1-(4-Chloro-phenylsulfanyl)-4-hydroxy-isoquinoline-3-carbonyl]-amino}-propionic acid; [(4-Hydroxy-7-phenylsulfanyl-isoquinoline-3-carbonyl)-amino]-acetic acid; [(4-Hydroxy-6-phenylsulfanyl-isoquinoline-3-carbonyl)-amino]-acetic acid; [(1-Chloro-4-hydroxy-7-phenylsulfanyl-isoquinoline-3-carbonyl)-amino]-acetic acid; [(1-Chloro-4-hydroxy-6-phenylsulfanyl-isoquinoline-3-carbonyl)-amino]-acetic acid; [(1-Bromo-4-hydroxy-7-phenylsulfanyl-isoquinoline-3-carbonyl)-amino]-acetic acid; [(1-Bromo-4-hydroxy-6-phenylsulfanyl-isoquinoline-3-carbonyl)-amino]-acetic acid; [(4-Hydroxy-7-phenoxy-isoquinoline-3-carbonyl)-amino]-acetic acid; [(4-Hydroxy-6-phenoxy-isoquinoline-3-carbonyl)-amino]-acetic acid; [(1-Chloro-4-hydroxy-7-phenoxy-isoquinoline-3-carbonyl)-amino]-acetic acid; [(1-Chloro-4-hydroxy-6-phenoxy-isoquinoline-3-carbonyl)-amino]-acetic acid; [(1-Bromo-4-hydroxy-7-phenoxy-isoquinoline-3-carbonyl)-amino]-acetic acid; [(1-bromo-4-hydroxy-6-phenoxy-isoquinoline-3-carbonyl)-amino]-acetic acid; {[7-(2,6-Dimethyl-phenoxy)-4-hydroxy-isoquinoline-3-carbonyl]-amino}-acetic acid; {[1-Chloro-7-(2,6-dimethyl-phenoxy)-4-hydroxy-isoquinoline-3-carbonyl]-amino}-acetic acid; {[1-bromo-7-(2,6-dimethyl-phenoxy)-4-hydroxy-isoquinoline-3-carbonyl]-amino}-acetic acid; [(1-bromo-7-chloro-4-hydroxy-isoquinoline-3-carbonyl)-amino]-acetic acid; [(1-bromo-6-Chloro-4-hydroxy-isoquinoline-3-carbonyl)-amino]-acetic acid; [(1-Bromo-4-hydroxy-7-trifluoromethyl-isoquinoline-3-carbonyl)-amino]-acetic acid; [(1-Bromo-4-hydroxy-6-trifluoromethyl-isoquinoline-3-carbonyl)-amino]-acetic acid; [(4-Hydroxy-1-phenoxy-isoquinoline-3-carbonyl)-amino]-acetic acid; [(1,7-dibromo-4-hydroxy-isoquinoline-3-carbonyl)-amino]-acetic acid; [(7-bromo-1-chloro-4-hydroxy-isoquinoline-3-carbonyl)-amino]-acetic acid; [(6-Bromo-4-hydroxy-isoquinoline-3-carbonyl)-amino]-acetic acid; [(1-bromo-7-fluoro-4-hydroxy-isoquinoline-3-carbonyl)-amino]-acetic acid; [(7-Fluoro-4-hydroxy-isoquinoline-3-carbonyl)-amino]-acetic acid; [(1-Chloro-7-fluoro-4-hydroxy-isoquinoline-3-carbonyl)-amino]-acetic acid; [(1-Chloro-4-hydroxy-benzo[g]isoquinoline-3-carbonyl)-amino]-acetic acid; [(1-Bromo-4-hydroxy-isoquinoline-3-carbonyl)-amino]-acetic acid; [(4-Hydroxy-6-phenyl-isoquinoline-3-carbonyl)-amino]-acetic acid; [(4-Hydroxy-7-phenyl-isoquinoline-3-carbonyl)-amino]-acetic acid; [(1-Chloro-4-hydroxy-6-phenyl-isoquinoline-3-carbonyl)-amino]-acetic acid; [(1-Chloro-4- hydroxy-7-phenyl-isoquinoline-3-carbonyl)-amino]-acetic acid; [(1-Bromo-4-hydroxy-6-phenyl-isoquinoline-3-carbonyl)-amino]-acetic acid; [(1-Bromo-4-hydroxy-7-phenyl-isoquinoline-3-carbonyl)-amino]-acetic acid; [(4-Hydroxy-5-phenyl-isoquinoline-3-carbonyl)-amino]-acetic acid; [(4-Hydroxy-8-phenyl-isoquinoline-3-carbonyl)-amino]-acetic acid; [(1-Chloro-4-hydroxy-5-phenyl-isoquinoline-3-carbonyl)-amino]-acetic acid; [(1-Chloro-4-hydroxy-8-phenyl-isoquinoline-3-carbonyl)-amino]-acetic acid; [(1-bromo-4-hydroxy-5-phenyl-isoquinoline-3-carbonyl)-amino]-acetic acid; [(1-Bromo-4-hydroxy-8-phenyl-isoquinoline-3-carbonyl)-amino]-acetic acid; [(1-Ethylsulfanyl-4-hydroxy-isoquinoline-3-carbonyl)-amino]-acetic acid; {[4-Hydroxy-1-(4-methoxy-phenylsulfanyl)-isoquinoline-3-carbonyl]-amino}-acetic acid; [(1-Chloro-4-hydroxy-7-iodo-isoquinoline-3-carbonyl)-amino]-acetic acid; [(1-Chloro-4-hydroxy-6-iodo-isoquinoline-3-carbonyl)-amino]-acetic acid; [(4-Hydroxy-7-iodo-isoquinoline-3-carbonyl)-amino]-acetic acid; [(1-Bromo-4-hydroxy-7-methyl-isoquinoline-3-carbonyl)-amino]-acetic acid; [(1-bromo-7-butoxy-4-hydroxy-isoquinoline-3-carbonyl)-amino]-acetic acid; [(1-bromo-6-butoxy-4-hydroxy-isoquinoline-3-carbonyl)-amino]-acetic acid; [(6-Benzyloxy-1-chloro-4-hydroxy-isoquinoline-3-carbonyl)-methyl-amino]-acetic acid; [(1-Chloro-4-hydroxy-isoquinoline-3-carbonyl)-methyl-amino]-acetic acid; [(1-Chloro-4-hydroxy-6-isopropoxy-isoquinoline-3-carbonyl)-methyl-amino]-acetic acid; [(1-Chloro-4-hydroxy-7-isopropoxy-isoquinoline-3.carbonyl)-methyl-amino]-acetic acid; [Carboxymethyl-(1-chloro-4-hydroxy-isoquinoline-3-carbonyl)-amino]-acetic acid; [Carboxymethyl-( 1-chloro-4-hydroxy-6-isopropoxy-isoquinoline-3-carbonyl)-amino]-acetic acid; 1-Chloro-4-hydroxy-isoquinoline-3-carboxylic acid (2-amino-ethyl)-amide (trifluoro-acetic acid salt); 1-Chloro-4-hydroxy-isoquinoline-3-carboxylic acid (2-methoxy-ethyl)-amide; 1-Chloro-4-hydroxy-isoquinoline-3-carboxylic acid (2-hydroxy-ethyl)-amide; 1-Chloro-4-hydroxy-isoquinoline-3-carboxylic acid (2-dimethylamino-ethyl)-amide; 1-Chloro-4-hydroxy-isoquinoline-3-carboxylic acid (2-acetylamino-ethyl)-amide; 1-Chloro-4-hydroxy-6-isopropoxy-isoquinoline-3-carboxylic acid (2-hydroxy-ethyl)-amide; 1-Chloro-4-hydroxy-6-isopropoxy-isoquinoline-3-carboxylic acid (2-methoxy-ethyl)-amide; 1-Chloro-4-hydroxy-6-isopropoxy-isoquinoline-3-carboxylic acid (2-amino-ethyl)-amide (trifluoro-acetic acid salt); 1-Chloro-4-hydroxy-6-isopropoxy-isoquinoline-3-carboxylic acid (2-dimethylamino-ethyl)-amide; 1-Chloro-4-hydroxy-7-isopropoxy-isoquinoline-3-carboxylic acid (2-amino-ethyl)-amide (trifluoro-acetic acid salt); 1-Chloro-4-hydroxy-7-isopropoxy-isoquinoline-3-carboxylic acid (2-methoxy-ethyl)-amide; 1-Chloro-4-hydroxy-7-isopropoxy-isoquinoline-3-carboxylic acid (2-dimethylamino-ethyl)-amide; 1-Chloro-4-hydroxy-7-isopropoxy-isoquinoline-3-carboxylic acid (2-hydroxy-ethyl)-amide; (S)-2-[(6-Benzyloxy-1-chloro-4-hydroxy-isoquinoline-3-carbonyl)-amino]-propionic acid; (R)-2-[(1-Chloro-4-hydroxy-isoquinoline-3-carbonyl)-amino]-3-hydroxy-propionic acid; (S)-2-[(1-Chloro-4-hydroxy-isoquinoline-3-carbonyl)-amino]-3-hydroxy-propionic acid; (R)-2-[(1-Chloro-4-hydroxy-6-isopropoxy-isoquinoline-3-carbonyl)-amino]-3-hydroxy-propionic acid; (S)-2-[(1-Chloro-4-hydroxy-6-isopropoxy-isoquinoline-3-carbonyl)-amino]-3-hydroxy-propionic acid; (R)-2-[(1-Chloro-4-hydroxy-7-isopropoxy-isoquinoline-3-carbonyl)-amino]-3-hydroxy-propionic acid; (S)-2-[(1-Chloro-4-hydroxy-7-isopropoxy-isoquinoline-3-carbonyl)-amino]-3-hydroxy-propionic acid; 2-[(1-Chloro-4-hydroxy-isoquinoline-3-carbonyl)-amino]-2-methyl-propionic acid; 2-[(1-Chloro-4-hydroxy-6-isopropoxy-isoquinoline-3-carbonyl)-amino]-2-methyl-propionic acid; (R)-2-[(1-Chloro-4-hydroxy-isoquinoline-3-carbonyl)-amino]-3-(1H-imidazol-4-yl)-propionic acid (trifluoro-acetic acid salt); (S)-2-[( 1-Chloro-4-hydroxy-isoquinoline-3-carbonyl)-amino]-3-(1 H-imidazol-4-yl)-propionic acid (trifluoro-acetic acid salt); (R)-2-[(1-Chloro-4-hydroxy-isoquinoline-3-carbonyl)-amino]-3-methyl-butyric acid; (S)-2-[(1-Chloro-4-hydroxy-isoquinoline-3-carbonyl)-amino]-3-methyl-butyric acid; (R)-2-[(1-Chloro-4-hydroxy-6-isopropoxy-isoquinoline-3-carbonyl)-amino]-3-methyl-butyric acid; (S)-2-[(1-Chloro-4-hydroxy-6-isopropoxy-isoquinoline-3-carbonyl)-amino]-3-methyl-butyric acid; (R)-2-[(1-Chloro-4-hydroxy-7-isopropoxy-isoquinoline-3-carbonyl)-amino]-3-methyl-butyric acid; (S)-2-[(1-Chloro-4-hydroxy-7-isopropoxy-isoquinoline-3-carbonyl)-amino]-3-methyl-butyric acid; (S)-2-[(6-Benzyloxy-1-chloro-4-hydroxy-isoquinoline-3-carbonyl)-amino]-3-methyl-butyric acid; (R)-2-[(1-Chloro-4-hydroxy-isoquinoline-3-carbonyl)-amino]-3-phenyl-propionic acid; (S)-2-[(1-Chloro-4-hydroxy-isoquinoline-3-carbonyl)-amino]-3-phenyl-propionic acid; (R)-2-[(1-Chloro-4-hydroxy-6-isopropoxy-isoquinoline-3-carbonyl)-amino]-3-phenyl-propionic acid; (S)-2-[(1-Chloro-4-hydroxy-6-isopropoxy-isoquinoline-3-carbonyl)-amino]-3-phenyl-propionic acid; (R)-2-[(1-Chloro-4-hydroxy-7-isopropoxy-isoquinoline-3-carbonyl)-amino]-3-phenyl-propionic acid; (S)-2-[(1-Chloro-4-hydroxy-7-isopropoxy-isoquinoline-3-carbonyl)-amino]-3-phenyl-propionic acid; (R)-2-[(1-Chloro-4-hydroxy-isoquinoline-3-carbonyl)-amino]-3-(4-hydroxy-phenyl)-propionic acid; (S)-2-[(1-Chloro-4-hydroxy-isoquinoline-3-carbonyl)-amino]-3-(4-hydroxy-phenyl)-propionic acid; (R)-2-[(1-Chloro-4-hydroxy-6-isopropoxy-isoquinoline-3-carbonyl)-amino]-3-(4-hydroxy-phenyl)-propionic acid; (S)-2-[( 1-Chloro-4-hydroxy-6-isopropoxy-isoquinoline-3-carbonyl)-amino]-3-(4-hydroxy-phenyl)-propionic acid; (R)-2-[(1-Chloro-4-hydroxy-7-isopropoxy-isoquinoline-3-carbonyl)-amino]-3-4-hydroxy-phenyl)-propionic acid; (S)-2-[(1-Chloro-4-hydroxy-7-isopropoxy-isoquinoline-3-carbonyl)-amino]-3-(4-hydroxy-phenyl)-propionic acid; (R)-2-[(1-Chloro-4-hydroxy-6-isopropoxy-isoquinoline-3-carbonyl)-amino]-pentanoic acid; (S)-2-[(1-Chloro-4-hydroxy-6-isopropoxy-isoquinoline-3-carbonyl)-amino]-pentanoic acid; (R)-1-(1-Chloro-4-hydroxy-isoquinoline-3-carbonyl)-pyrrolidine-2-carboxylic acid; (S)-1-(1-Chloro-4-hydroxy-isoquinoline-3-carbonyl)-pyrrolidine-2-carboxylic acid; (R)-1-(1-Chloro-4-hydroxy-6-isopropoxy-isoquinoline-3-carbonyl)-pyrrolidine-2-carboxylic acid; (S)-1-(1-Chloro-4-hydroxy-6-isopropoxy-isoquinoline-3-carbonyl)-pyrrolidine-2-carboxylic acid; (R)-6-Amino-2-[(1-Chloro-4-hydroxy-isoquinoline-3-carbonyl)-amino]-hexanoic acid (trifluoro-acetic acid salt); (S)-6-Amino-2-[(1-Chloro-4-hydroxy-isoquinoline-3-carbonyl)-amino]-hexanoic acid (trifluoro-acetic acid salt); (R)-6-Amino-2-[(1-chloro-4-hydroxy-6-isopropoxy-isoquinoline-3-carbonyl)-amino]-hexanoic acid; trifluoroacetic acid salt; (S)-6-Amino-2-[(1-chloro-4-hydroxy-6-isopropoxy-isoquinoline-3-carbonyl)-amino]-hexanoic acid (trifluoro-acetic acid salt); (R)-6-Amino-2-[(1-chloro-4-hydroxy-7-isopropoxy-isoquinoline-3-carbonyl)-amino]-hexanoic acid; trifluoroacetic acid salt; (S)-6-Amino-2-[(1-chloro-4-hydroxy-7-isopropoxy-isoquinoline-3-carbonyl)-amino]-hexanoic acid (trifluoro-acetic acid salt); (R)-2-[(1-Chloro-4-hydroxy-isoquinoline-3-carbonyl)-amino]-succinic acid; (S)-2-[(1-Chloro-4-hydroxy-isoquinoline-3-carbonyl)-amino]-succinic acid; (R)-2-[(1-Chloro-4-hydroxy-6-isopropoxy-isoquinoline-3-carbonyl)-amino]-succinic acid; (S)-2-[(1-Chloro-4-hydroxy-6-isopropoxy-isoquinoline-3-carbonyl)-amino]-succinic acid; (R)-2-[(1-Chloro-4-hydroxy-7-isopropoxy-isoquinoline-3-carbonyl)-amino]-succinic acid; 1-[(1-Chloro-4-hydroxy-isoquinoline-3-carbonyl)-amino]-cyclopropanecarboxylic acid; l-[(1-Chloro-4-hydroxy-6-isopropoxy-isoquinoline-3-carbonyl)-amino]-cyclopropanecarboxylic acid; Dideutero-[(1-chloro-4-hydroxy-isoquinoline-3-carbonyl)-amino]-acetic acid; (R)-2-[(6-Benzyloxy-1-chloro-4-hydroxy-isoquinoline-3-carbonyl)-amino]-propionic acid; (S)-2-[(7-Benzyloxy-1-chloro-4-hydroxy-isoquinoline-3-carbonyl)-amino]-propionic acid; (R)-2-[(7-Benzyloxy-1-chloro-4-hydroxy-isoquinoline-3-carbonyl)-amino]-propionic acid; (S)-2-[(1-Chloro-4-hydroxy-isoquinoline-3-carbonyl)-amino]-propionic acid; (R)-2-[(1-Chloro-4-hydroxy-isoquinoline-3-carbonyl)-amino]-propionic acid; (S)-2-[(6-Isopropoxy-1-chloro-4-hydroxy-isoquinoline-3-carbonyl)-amino]-propionic acid; (R)-2-[6-Isopropoxy-1-chloro-4-hydroxy-isoquinoline-3-carbonyl)-amino]-propionic acid; (S)-2-[(7-Isopropoxy-1-chloro-4-hydroxy-isoquinoline-3-carbonyl)-amino-propionic acid; (R)-2-[(7-Isopropoxy-1-chloro-4-hydroxy-isoquinoline-3-carbonyl)-amino]propionic acid; l-Chloro-4-hydroxy-6-isopropoxy-isoquinoline-3-carboxylic acid (2-hydroxy-1-hydroxymethyl-ethyl)-amide; I-Chloro-4-hydroxy-7-isopropoxy-isoquinoline-3-carboxylic acid (2-hydroxy-1-hydroxymethyl-ethyl)-amide; 1-Chloro-4-hydroxy-isoquinoline-3-carboxylic acid (2-hydroxy-1-hydroxymethyl-ethyl)-amide; {[7-(3,5-Difluoro-phenoxy)-4-hydroxy-isoquinoline-3-carbonyl]-amino}-acetic acid; {[6-(3,5-Difluoro-phenoxy)-4-hydroxy-isoquinoline-3-carbonyl]-amino}-acetic acid; ({7-[4-(4-Fluoro-phenoxy)-phenoxy]4-hydroxy-isoquinoline-3-carbonyl}-amino)-acetic acid; ({6-[4-(4-Fluoro-phenoxy)-phenoxy]-4-hydroxy-isoquinoline-3-carbonyl}-amino)-acetic acid; {[7-(3-Chloro-4-fluoro-phenoxy)4-hydroxy-isoquinoline-3-carbonyl]-amino}-acetic acid; {[6-(3-Chloro-4-fluoro-phenoxy)-4-hydroxy-isoquinoline-3-carbonyl]-amino}-acetic acid; (S)-2-{[7-(3-Fluoro-5-methoxy-phenoxy)-4-hydroxy-isoquinoline-3-carbonyl]-amino}-propionic acid; 2-(S)-[(7-Cyclohexyloxy-4-hydroxy-isoquinoline-3-carbonyl)-amino]-propionic acid; 2-(S)-{[7-(4-Fluoro-phenoxy)-4-hydroxy-1-methyl-isoquinoline-3-carbonyl]-amino}-propionic acid; 2-(S)-{[7-(4-Fluoro-phenoxy)-4-hydroxy-isoquinoline-3-carbonyl]-amino}-propionic acid; 2-(S)-[(4-Hydroxy-1-methyl-7-phenoxy-isoquinoline-3-carbonyl)-amino]-propionic acid; 2-(S)-[(4-Hydroxy-1-methyl-7-phenylsulfanyl-isoquinoline-3-carbonyl)-amino]-propionic acid; 2-(S)-{[4-Hydroxy-7-(4-trifluoromethyl-phenoxy)-isoquinoline-3-carbonyl]-amino}-propionic acid; {[7-(4-Chloro-phenoxy)-4-hydroxy-1-methyl-isoquinoline-3c-carbonyl]-amino-acetic acid; {[6-(4-Chloro-phenoxy)-4-hydroxy-1-methyl-isoquinoline-3-carbonyl]-amino}-acetic acid; {[7-(3,5-Difluoro-phenoxy)4-hydroxy-1-methyl-isoquinoline-3-carbonyl]-amino}-acetic acid; {[4-Hydroxy-7-(4-methoxy-phenoxy)-1-methyl-isoquinoline-3-carbonyl]-amino}-acetic acid; {[4-Hydroxy-6-(4-methoxy-phenoxy)-1-methyl-isoquinoline-3-carbonyl]-amino}-acetic acid; [(6-Cyclohexyloxy-4-hydroxy-isoquinoline-3-carbonyl)-amino]-acetic acid; [(7-Cyclohexyloxy-4-hydroxy-isoquinoline-3-carbonyl)-amino]-acetic acid; [(7-Cyclohexyloxy-4-hydroxy-1-methyl-isoquinoline-3-carbonyl)-amino]-acetic acid; [(7-Cyclohexylsulfanyl-4-hydroxy-isoquinoline-3-carbonyl)-amino]-acetic acid; [(7-Cyclohexanesulfonyl-4-hydroxy-isoquinoline-3-carbonyl)-amino]-acetic acid; [(4-Hydroxy-1-isobutyl-isoquinoline-3-carbonyl)-amino]-acetic acid; [(4-Hydroxy-1-pyridin-2-yl-isoquinoline-3-carbonyl)-amino]-acetic acid; [(1-Ethyl-4-hydroxy-7-phenoxy-isoquinoline-3-carbonyl)-amino]-acetic acid; [(1-Dimethylaminomethyl-4-hydroxy-7-phenylsulfanyl-isoquinoline-3-carbonyl)-amino]-acetic acid; [(4-Hydroxy-1-methyl-7-phenylsulfanyl-isoquinoline-3-carbonyl)-amino]-acetic acid; {[4-Hydroxy-1-methyl-7-(4-trifluoromethyl-phenoxy)-isoquinoline-3-carbonyl]-amino}-acetic acid; and {[4-Hydroxy-7-phenoxy-1-(3-phenoxy-propyl)-isoquinoline-3-carbonyl]-amino}-acetic acid, {[4-Hydroxy-7-(4-hydroxy-phenoxy)-isoquinoline-3-carbonyl]-amino}-acetic acid, [(1-Benzoyl-4-hydroxy-7-phenoxy-isoquinoline-3-carbonyl)-amino]-acetic acid, {[4-Hydroxy-7-(4-hydroxy-phenoxy)-1-methyl-isoquinoline-3-carbonyl]-amino}-acetic acid, {[4-Hydroxy-7-(4-propoxy-phenoxy)-isoquinoline-3-carbonyl]-amino}-acetic acid, {[7-(2-Dimethylamino-benzooxazol-5-yloxy)-4-hydroxy-isoquinoline-3-carbonyl]-amino }-acetic acid, {[4-Hydroxy-7-(2-methyl-benzooxazol-6-yloxy)-isoquinoline-3-carbonyl]-amino}-acetic acid, ([4-Hydroxy-1-methyl-7-(2-methyl-benzooxazol-6-yloxy)-isoquinoline-3-carbonyl]-amino -acetic acid, ([7-(Benzo[1,3]dioxol-5-yloxy)-4-hydroxy-isoquinoline-3-carbonyl]-amino}-acetic acid, {[7-(2,3-Dihydro-benzofuran-5-yloxy)-4-hydroxy-isoquinoline-3-carbonyl]-amino}-acetic acid, {[4-Hydroxy-7-(4-methoxy-3,5-dimethyl-phenoxy)-isoquinoline-3-carbonyl]-amino}-acetic acid, {[7-(3-Chloro-4-methoxy-phenoxy)4-hydroxy-isoquinoline-3-carbonyl]-amino}-acetic acid, {[4-Hydroxy-7-(4-methoxy-3-methyl-phenoxy)-isoquinoline-3-carbonyl]-amino}-acetic acid, {[4-Hydroxy-7-(2-morpholin-4-yl-benzothiazol-6-yloxy)-isoquinoline-3-carbonyl]-amino}-acetic acid, {[1-(4-Fluoro-phenyl)-4-hydroxy-7-phenoxy-isoquinoline-3-carbonyl]-amino}-acetic acid, [(4-Hydroxy-8-phenoxy-isoquinoline-3-carbonyl)-amino]-acetic acid (Compound D), [(4-Hydroxy-1-methyl-8-phenoxy-isoquinoline-3-carbonyl)-amino]-acetic acid (Compound E), 4-Hydroxy-7-(2-methyl-benzothiazol-6-yloxy)-isoquinoline-3-carboxylic acid (2-oxo-propyl)-amide, {[4-Hydroxy-1-methyl-7-(2-methyl-benzothiazol-6-yloxy)-isoquinoline-3-carbonyl]-amino}-acetic acid, [(4-Hydroxy-7-phenoxy-1-thiophen-3-yl-isoquinoline-3-carbonyl)-amino]-acetic acid, {[4-Hydroxy-1-methyl-6-(2-methyl-benzothiazol-6-yloxy)-isoquinoline-3-carbonyl]-amino -acetic acid [(7-Chloro-4-hydroxy-1-methyl-isoquinoline-3-carbonyl)-amino]-acetic acid (Compound F), [(7-Cyclopentylsulfanyl-4-hydroxy-isoquinoline-3-carbonyl)-amino]-acetic acid, {[7-(2-Dimethylamino-benzothiazol-6-yloxy)-4-hydroxy-isoquinoline-3-carbonyl]-amino}-acetic acid, [(4-Hydroxy-7-phenoxy-1-trifluoromethyl-isoquinoline-3-carbonyl)-amino]-acetic acid, [(4-Hydroxy-7-phenoxy-1-phenyl-isoquinoline-3-carbonyl)-amino]-acetic acid, [(8-Chloro-4-hydroxy-1-phenyl-isoquinoline-3-carbonyl)-amino]-acetic acid, [(8-Chloro-4-hydroxy-1-methyl-isoquinoline-3-carbonyl)-amino]-acetic acid, {[7-(4-Benzyloxy-phenoxy)4-hydroxy-1-methyl-isoquinoline-3-carbonyl]-amino}-acetic acid, [(1-Butyl-4-Hydroxy-7-phenoxy-isoquinoline-3-carbonyl)-amino]-acetic acid, [(7-Benzyl-4-hydroxy-isoquinoline-3-carbonyl)-amino]-acetic acid, 2-[(4-Hydroxy-8-phenoxy-isoquinoline-3-carbonyl)-amino]-acrylic acid, {[8-(4-Fluoro-phenoxy)-4-hydroxy-isoquinoline-3-carbonyl]-amino}-acetic acid {[8-(4-Fluoro-phenoxy)-4-hydroxy-1-methyl-isoquinoline-3-carbonyl]-amino}-acetic acid (Compound G), {[1-Ethyl-8-(4-fluoro-phenoxy)-4-hydroxy-isoquinoline-3-carbonyl]-amino}-acetic acid, and pharmaceutically acceptable salts, esters and prodrugs thereof.

In certain embodiments, compounds used in the methods of the invention are selected from a compound of the formula II 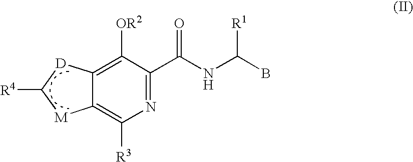
wherein
R¹ is selected from the group consisting of hydrogen, (C₁-C₆)-alkyl, (C₃-C₇)-cycloalkyl, aryl, or a substituent of the α-carbon atom of an α-amino acid, wherein the amino acid is a natural L-amino acid or its D-isomer;
B is —CO₂H or a CO₂-G carboxyl radical, where G is a radical of an alcohol G-OH in which G is selected from the group consisting of (C₁-C₂₀)-alkyl radical, (C₃-C₈) cycloalkyl radical, (C₂-C₂₀)-alkenyl radical, (C₃-C₈)-cycloalkenyl radical, retinyl radical, (C₂-C₂₀)-alkynyl radical, (C₄-C₂₀)-alkenynyl radical;
R² is selected from the group consisting of hydrogen, (C₁-C₁₀)-alkyl, (C₂-C₁₀)-alkenyl, (C₂-C₁₀)-alkynyl, wherein alkenyl or alkynyl contains one or two C—C multiple bonds; unsubstituted fluoroalkyl radical of the formula —[CH₂]ₓ—C_{f}H_{(2f+1-g)}—F_{g}, aryl, heteroaryl, and (C₇-C₁₁)-aralkyl;
one of D or M is —S—, and the other is ═C(R⁵)—;
R³, R⁴, and R⁵ are identical or different and are selected from the group consisting of hydrogen, hydroxyl, halogen, cyano, trifluoromethyl, nitro, carboxyl; (C₁-C₂₀)-alkyl, (C₃-C₈)-cycloalkyl, (C₃-C₈)-cycloalkoxy, (C₆-C₁₂)-aryl, (C₇-C₁₆)-aralkyl, (C₇-C₁₆)-aralkenyl, (C₇-C₁₆)-aralkynyl, (C₂-C₂₀)-alkenyl, (C₂-C₂₀)-alkynyl, (C₁-C₂₀)-alkoxy, (C₂-C₂₀)-alkenyloxy, (C₂-C₂₀)-alkynyloxy, retinyloxy, (C₆-C₁₂)-aryloxy, (C₇-C₁₆)-aralkyloxy, (C₁-C₁₆)-hydroxyalkyl, —O—[CH₂]ₓC_{f}H_{(2f+1-g)}F_{g}, —OCF₂Cl, —OCF₂—CHFCl, (C₁-C₂₀)-alkylcarbonyl, (C₃-C₈)-cycloalkylcarbonyl, (C₆-C₁₂)-arylcarbonyl, (C₇-C₁₆)-aralkylcarbonyl, cinnamoyl, (C₂-C₂₀)-alkenylcarbonyl, (C₂-C₂₀)-alkynylcarbonyl, (C₁-C₂₀)-alkoxycarbonyl, (C₆-C₁₂)-aryloxycarbonyl, (C₇-C₁₆)-aralkyloxycarbonyl, (C₃-C₈)-cycloalkoxycarbonyl, (C₂-C₂₀)-alkenyloxycarbonyl, retinyloxycarbonyl, (C₂-C₂₀)-alkynyloxycarbonyl, (C₁-C₁₂)-alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N—(C₁-C₁₂)-alkylcarbamoyl, N,N-di-(C₁-C₁₂)-alkylcarbamoyl, N—(C₃-C₈)-cycloalkylcarbamoyl, N,N-dicyclo-(C₃-C₈)-alkylcarbamoyl, N—(C₁-C₁₀)-alkyl-N—(C₃-C₈)-cycloalkylcarbamoyl, N—((C₃-C₈)-cycloalkyl-(C₁-C₆)-alkyl)-carbamoyl, N-(+)-dehydroabietylcarbamoyl, N—(C₁-C₆)-alkyl-N-(+)-dehydroabietylcarbamoyl, N—(C₆-C₁₂)-arylcarbamoyl, N—(C₇-C₁₆)-aralkylcarbamoyl, N—(C₁-C₁₀)-alkyl-N—(C₆-C₁₆)-arylcarbamoyl, N—(C₁-C₁₀)-alkyl-N—(C₇-C₁₆)-aralkylcarbamoyl, carbamoyloxy, N—(C₁-C₁₂)-alkylcarbamoyloxy, N,N-di-(C₁-C₁₂)-alkylcarbamoyloxy, N—(C₃-C₈)-cycloalkylcarbamoyloxy, N—(C₆-C₁₂)-arylcarbamoyloxy, N—(C₇-C₁₆)-aralkylcarbamoyloxy, N—(C₁-C₁₀)-alkyl-N—(C₆-C₁₂)-arylcarbamoyloxy, N—(C₁-C₁₀)-alkyl-N—(C₇-C₁₆)-aralkylcarbamoyloxy, N—((C₁-C₁₀)-alkyl)-carbamoyloxy, N—(C₁-C₁₀)-alkyl-N—((C₇-C₁₆)-aralkyloxy-(C₁-C₁₀)-alkyl)-carbamoyloxyamino, (C₁-C₁₂)-alkylamino, di-(C₁-C₁₂)-alkylamino, (C₃-C₈)-cycloalkylamino, (C₃-C₁₂)-alkenylamino, (C₃-C₁₂)-alkynylamino, N—(C₆-C₁₂)-arylamino, N—(C₇-C₁₁)-aralkylamino, N-alkyl-aralkylamino, N-alkyl-arylamino, (C₁-C₁₂)-alkoxyamino, (C₁-C₁₂)-alkoxy-N—(C₁-C₁₀)-alkylamino, (C₁-C₁₂)-alkanoylamino, (C₃-C₈)-cycloalkanoylamino, (C₆-C₁₂)-aroylamino, (C₇-C₁₆)-aralkanoylamino, (C₁-C₁₂)-alkanoyl-N—(C₁-C₁₀)-alkylamino, (C₃-C₈)-cycloalkanoyl-N—(C₁-C₁₀)-alkylamino, (C₆-C₁₂)-aroyl-N—(C₁-C₁₀)-alkylamino, (C₇-C₁₁)-aralkanoyl-N—(C₁-C₁₀)-alkylamino, amino-(C₁-C₁₀)-alkyl, (C₁-C₂₀)-alkylmercapto, (C₁-C₂₀)-alkylsulfinyl, (C₁-C₂₀)-alkylsulfonyl, (C₆-C₁₂)-arylmercapto, (C₆-C₁₂)-arylsulfinyl, (C₆-C₁₂)-arylsulfonyl, (C₇-C₁₆)-aralkylmercapto, (C₇-C₁₆)-aralkylsulfinyl, (C₇-C₁₆)-aralkylsulfonyl, sulfamoyl, N—(C₁-C₁₀)-alkylsulfamoyl, N,N-di-(C₁-C₁₀)-alkylsulfamoyl, (C₃-C₈)-cycloalkylsulfamoyl, N—(C₆-C₁₂)-arylsulfamoyl, N—(C₇-C₁₆)-aralkylsulfamoyl, N—(C₁-C₁₀)-alkyl-N—(C₆-C₁₂)-arylsulfamoyl, N—(C₁-C₁₀)-alkyl-N—(C₇-C₁₆)-aralkylsulfamoyl, (C₁-C₁₀)-alkylsulfonamido, (C₇-C₁₆)-aralkylsulfonamido, and N—((C₁-C₁₀)-alkyl-(C₇-C₁₆)-aralkylsulfonamido; where an aryl radical may be substituted by 1 to 5 substituents selected from hydroxyl, halogen, cyano, trifluoromethyl, nitro, carboxyl, (C₂-C₁₆)-alkyl, (C₃-C₈)-cycloalkyl, (C₃-C₈)-cycloalkoxy, (C₆-C₁₂)-aryl, (C₇-C₁₆)-aralkyl, (C₂-C₁₆)-alkenyl, (C₂-C₁₂)-alkynyl, (C₁-C₁₆)-alkoxy, (C₁-C₁₆)-alkenyloxy, (C₆-C₁₂)-aryloxy, (C₇-C₁₆)-aralkyloxy, (C₁-C₈)-hydroxyalkyl, —O—[CH₂]ₓC_{f}H_{(2f+1-g)}F_{g}, —OCF₂Cl, and —OCF₂—CHFCl;
x is 0 to 3;
f is 1 to 8; and
g is o or 1 to (2f+1);
including the physiologically active salts and prodrugs derived therefrom.

Compounds of Formula (II) include, but are not limited to, [(2-bromo-4-hydroxy-thieno[2,3-c]pyridine-5-carbonyl)-amino]-acetic acid, [(2-bromo-7-hydroxy-thieno[3,2-c]pyridine-6-carbonyl)-amino]-acetic acid, {[4-hydroxy-2-(4-methoxy-phenyl)-thieno[2,3-c]pyridine-5-carbonyl]-amino}-acetic acid, {[7-hydroxy-2-(4-methoxy-phenyl)-thieno[3,2-c]pyridine-6-carbonyl]-amino }-acetic acid, [(4-hydroxy-2,7-dimethyl-thieno[2,3-c]pyridine-5-carbonyl)-amino]-acetic acid, [(7-hydroxy-2,4-dimethyl-thieno[3,2-c]pyridine-6-carbonyl)-amino]-acetic acid, {[7-hydroxy-4-methyl-2-(4-phenoxy-phenyl)-thieno[3,2-c]pyridine-6-carbonyl]-amino}-acetic acid, {[4-hydroxy-2-(4-phenoxy-phenyl)-7-methyl-thieno[2,3-c]pyridine-5-carbonyl]-amino}-acetic acid, {[4-hydroxy-2-(4-phenoxy-phenyl)-thieno[2,3-c]pyridine-5-carbonyl]-amino}-acetic acid, {[7-hydroxy-2-(4-phenoxy-phenyl)-thieno[3,2-c]pyridine-6-carbonyl]-amino-acetic acid, [(2,7-dibromo-4-hydroxy-thieno[2,3-c]pyridine-5-carbonyl)-amino]-acetic acid, [(2-bromo-7-chloro-4-hydroxy-thieno[2,3-c]pyridine-5-carbonyl)-amino]-acetic acid, [(7-hydroxy-thieno[3,2-c]pyridine-6-carbonyl)-amino]-acetic acid, [(4-hydroxy-thieno[2,3-c]pyridine-5-carbonyl)-amino]-acetic acid, [(2-bromo-4-Chloro-7-hydroxy-thieno[3,2-c]pyridine-6-carbonyl)-amino]-acetic acid, [(2,4-dibromo-7-hydroxy-thieno[3,2-c]pyridine-6-carbonyl)-amino]-acetic acid, [(7-hydroxy-2-phenylsulfanyl-thieno[3,2-c]pyridine-6-carbonyl)-amino]-acetic acid, [(4-hydroxy-2-phenylsulfanyl-thieno[2,3-c]pyridine-5-carbonyl)-amino]-acetic acid, [(4-hydroxy-2,7-diphenyl-thieno[2,3-c]pyridine-5-carbonyl)-amino]-acetic acid, [(7-hydroxy-2,4-diphenyl-thieno[3,2-c]pyridine-6-carbonyl)-amino]-acetic acid, [(7-hydroxy-2-styryl-thieno[3,2-c]pyridine-6-carbonyl)-amino]-acetic acid, [(7-hydroxy-2-phenoxy-thieno[3,2-c]pyridine-6-carbonyl)-amino]-acetic acid, [(7-hydroxy-2-phenethyl-thieno[3,2-c]pyridine-6-carbonyl)-amino]-acetic acid, {[7-hydroxy-2-(3-trifluoromethyl-phenyl)-thieno[3,2-c]pyridine-6-carbonyl]-amino}-acetic acid, {[4-bromo-7-hydroxy-2-(3-trifluoromethyl-phenyl)-thieno[3,2-c]pyridine-6-carbonyl]-amino}-acetic acid, {[4-cyano-7-hydroxy-2-(3-trifluoromethyl-phenyl)-thieno[3,2-c]pyridine-6-carbonyl]-amino}-acetic acid, [(2-cyano-7-hydroxy-thieno[3,2-c]pyridine-6-carbonyl)-amino]-acetic acid, {[7-hydroxy-2-(4-trifluoromethyl-phenyl)-thieno[3,2-c]pyridine-6-carbonyl)-amino}-acetic acid, {[7-hydroxy-2-(2-trifluoromethyl-phenyl)-thieno[3,2-c]pyridine-6-carbonyl]-amino) -acetic acid, {[4-bromo-3-(4-fluoro-phenyl)-7-hydroxy-thieno[3,2-c]pyridine-6-carbonyl]-amino}-acetic acid, {[3-(4-fluoro-phenyl)-7-hydroxy-thieno[3,2-c]pyridine-6-carbonyl]-amino}-acetic acid, {[3-(4-fluoro-phenyl)-7-hydroxy-4-methyl-thieno[3,2-c]pyridine-6-carbonyl]-amino}-acetic acid, {[4-cyano-3-(4-fluoro-phenyl)-7-hydroxy-thieno[3,2-c]pyridine-6-carbonyl]-amino}-acetic acid, {[2-(4-fluoro-phenyl)-7-hydroxy-thieno[3,2-c]pyridine-6-carbonyl]-amino}-acetic acid, {[2-(4-fluoro-phenyl)-7-hydroxy-4-methyl-thieno[3,2-c]pyridine-6-carbonyl]-amino}-acetic acid, {[2,3-bis-(4-fluoro-phenyl)-7-hydroxy-thieno[3,2-c]pyridine-6-carbonyl]-amino}-acetic acid, {[7-bromo-3-(4-fluoro-phenyl)-4-hydroxy-thieno[2,3-c]pyridine-5-carbonyl]-amino}-acetic acid, {[3-(4-fluoro-phenyl)-4-hydroxy-thieno[2,3-c]pyridine-5-carbonyl]-amino}-acetic acid, {[2-(4-fluoro-phenyl)-4-hydroxy-thieno[2,3-c]pyridine-5-carbonyl]-amino}-acetic acid, {[2-(4-fluoro-phenyl)-4-hydroxy-7-methyl-thieno[2,3-c]pyridine-5-carbonyl]-amino}-acetic acid, [(7-chloro-4-hydroxy-thieno[2,3-c]pyridine-S-carbonyl)-amino]-acetic acid, [(4-Chloro-7-hydroxy-thieno[3,2-c]pyridine-6-carbonyl)-amino]-acetic acid, [(7-bromo-4-hydroxy-thieno[2,3-c]pyridine-5-carbonyl)-amino]-acetic acid, [(4-bromo-7-hydroxy-thieno[3,2-c]pyridine-6-carbonyl)-amino]-acetic acid, [(4-hydroxy-7-phenyl-thieno[2,3-c]pyridine-5-carbonyl)-amino]-acetic acid, [(7-hydroxy-4-phenyl-thieno[3,2-c]pyridine-6-carbonyl)-amino]-acetic acid, [(4-cyano-7-hydroxy-thieno[3,2-c]pyridine-6-carbonyl)-amino]-acetic acid (Compound H), {[7-(4-fluoro-phenyl)-4-hydroxy-thieno[2,3-c]pyridine-5-carbonyl]-amino}-acetic acid, {[4-(4-fluoro-phenyl)-7-hydroxy-thieno[3,2-c]pyridine-6-carbonyl]-amino}-acetic acid, 2-(7-(furan-2-yl)-4-hydroxythieno[2,3-c]pyridine-5-carboxamido)acetic acid, [(4-furan-2-yl-7-hydroxy-thieno[3,2-c]pyridine-6-carbonyl)-amino]-acetic acid, [(7-furan-3-yl-4-hydroxy-thieno[2,3-c]pyridine-5-carbonyl)-amino]-acetic acid, [(4-furan-3-yl-7-hydroxy-thieno[3,2-c]pyridine-6-carbonyl)-amino]-acetic acid, 2-(4-hydroxy-7-(thiophen-2-yl)thieno[2,3-c]pyridine-5-carboxamido)acetic acid, [(7-hydroxy-4-thiophen-2-yl-thieno[3,2-c]pyridine-6-carbonyl)-amino]-acetic acid, [(4-hydroxy-7-thiophen-3-yl-thieno[2,3-c]pyridine-5-carbonyl)-amino]-acetic acid, [(7-hydroxy-4-thiophen-3-yl-thieno[3,2-c]pyridine-6-carbonyl)-amino]-acetic acid, [(4-hydroxy-7-methyl-thieno[2,3-c]pyridine-5-carbonyl)-amino]-acetic acid, [(7-hydroxy-4-methyl-thieno[3,2-c]pyridine-6-carbonyl)-amino]-acetic acid, [(7-ethynyl-4-hydroxy-thieno[2,3-c]pyridine-5-carbonyl)-amino]-acetic acid, [(4-ethynyl-7-hydroxy-thieno[3,2-c]pyridine-6-carbonyl)-amino]-acetic acid, [(7-cyano-4-hydroxy-thieno[2,3-c]pyridine-5-carbonyl)-amino]-acetic acid, and pharmaceutically acceptable salts, esters and prodrugs thereof.

Particularly preferred compounds for use in the present invention include [(1-Chloro-4-hydroxy-isoquinoline-3-carbonyl)-amino]-acetic acid (Compound A), [(4-Hydroxy-1-methyl-7-phenoxy-isoquinoline-3-carbonyl)-amino]-acetic acid (Compound B), {[4-Hydroxy-7-(4-methoxy-phenoxy)-isoquinoline-3-carbonyl]-amino}-acetic acid (Compound C). [(4-Hydroxy-8-phenoxy-isoquinoline-3-carbonyl)-amino]-acetic acid (Compound D), [(4-Hydroxy-1-methyl-8-phenoxy-isoquinoline-3-carbonyl)-amino]-acetic acid (Compound E), [(7-Chloro-4-hydroxy-1-methyl-isoquinoline-3-carbonyl)-amino]-acetic acid (Compound F), {[8-(4-Fluoro-phenoxy)-4-hydroxy-1-methyl-isoquinoline-3-carbonyl]-amino}-acetic acid (Compound G), and [(4-cyano-7-hydroxy-thieno[3,2-c]pyridine-6-carbonyl)-amino]-acetic acid (Compound H).

As used herein, “alkyl” refers to monovalent alkyl groups having from 1 to 10 carbon atoms, preferably from 1 to 5 carbon atoms and more preferably 1 to 3 carbon atoms. This term is exemplified by groups such as methyl, ethyl, n-propyl, iso-propyl, n-butyl, t-butyl, n-pentyl and the like.

“Substituted alkyl” refers to an alkyl group, of from 1 to 10 carbon atoms, preferably, 1 to 5 carbon atoms, having from 1 to 5 substituents, preferably 1 to 3 substituents, independently selected from the group consisting of alkoxy, substituted alkoxy, acyl, acylamino, acyloxy, amino, substituted amino, aminoacyl, aminocarbonylamino, aminothiocarbonylamino, aminocarbonyloxy, aryl, substituted aryl, aryloxy, substituted aryloxy, aryloxyaryl, substituted aryloxyaryl, cyano, halogen, hydroxyl, nitro, oxo, thioxo, carboxyl, carboxyl esters, cycloalkyl, substituted cycloalkyl, thiol, alkylthio, substituted alkylthio, arylthio, substituted arylthio, cycloalkylthio, substituted cycloalkylthio, heteroarylthio, substituted heteroarylthio, heterocyclicthio, substituted heterocyclicthio, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —OS(O)₂-alkyl, —OS(O)₂-substituted alkyl, —OS(O)₂-aryl, —OS(O)₂-substituted aryl, OS(O)₂-heteroaryl, —OS(O)₂-substituted heteroaryl, —OS(O)₂-heterocyclic, —OS(O)₂-substituted heterocyclic, —OSO₂—NR⁴⁰R⁴⁰ where each R⁴⁰ is hydrogen or alkyl, —NR⁴⁰S(O)₂-alkyl, —NR⁴⁰S(O)₂-substituted alkyl,-NR⁴⁰S(O)₂-aryl, —NR⁴⁰S(O)₂-substituted aryl, —NR⁴⁰S(O)₂-heteroaryl, —NR⁴⁰S(O)₂-substituted heteroaryl, —NR⁴⁰S(O)₂—heterocyclic, —NR⁴⁰S(O)₂-substituted heterocyclic, —NR⁴⁰S(O)₂—NR⁴⁰-alkyl, —NR⁴⁰S(O)₂—NR⁴⁰-substituted alkyl, —NR⁴⁰S(O)₂—NR⁴⁰-aryl, —NR⁴⁰S(O)₂—NR⁴⁰-substituted aryl, —NR⁴⁰S(O)₂—NR⁴⁰-heteroaryl, —NR⁴⁰S(O)₂—NR⁴⁰-substituted heteroaryl, —NR⁴⁰S(O)₂—NR⁴⁰-heterocyclic, and —NR⁴⁰S(O)₂—NR⁴⁰-substituted heterocyclic where each R is hydrogen or alkyl.

“Alkoxy” refers to the group “alkyl-O—” which includes, by way of example, methoxy, ethoxy, n-propoxy, iso-propoxy, n-butoxy, t-butoxy, sec-butoxy, n-pentoxy and the like.

“Substituted alkoxy” refers to the group “substituted alkyl-O—”.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, aryl-C(O)—, substituted aryl-C(O)—, heteroaryl-C(O)—, substituted heteroaryl-C(O), heterocyclic-C(O)—, and substituted heterocyclic-C(O)— provided that a nitrogen atom of the heterocyclic or substituted heterocyclic is not bound to the —C(O)— group wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

The term “aminoacyl” or as a prefix “carbamoyl” or “carboxamide” or “substituted carbamoyl” or “substituted carboxamide” refers to the group —C(O)NR⁴²R⁴² where each R⁴² is independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic and where each R⁴² is joined to form together with the nitrogen atom a heterocyclic or substituted heterocyclic wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein. “Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein. “Alkenyl” refers to alkenyl group preferably having from 2 to 6 carbon atoms and more preferably 2 to 4 carbon atoms and having at least 1 and preferably from 1 to 2 sites of alkenyl unsaturation. “Substituted alkenyl” refers to alkenyl groups having from 1 to 3 substituents, and preferably 1 to 2 substituents, selected from the group consisting of alkoxy, substituted alkoxy, acyl, acylamino, acyloxy, amino, substituted amino, aminoacyl, aryl, substituted aryl, aryloxy, substituted aryloxy, cyano, halogen, hydroxyl, nitro, carboxyl, carboxyl esters, cycloalkyl, substituted cycloalkyl, heteroaryl, substituted heteroaryl, heterocyclic, and substituted heterocyclic.

“Alkynyl” refers to alkynyl group preferably having from 2 to 6 carbon atoms and more preferably 2 to 3 carbon atoms and having at least 1 and preferably from 1-2 sites of alkynyl unsaturation.

“Substituted alkynyl” refers to alkynyl groups having from 1 to 3 substituents, and preferably 1 to 2 substituents, selected from the group consisting of alkoxy, substituted alkoxy, acyl, acylamino, acyloxy, amino, substituted amino, aminoacyl, aryl, substituted aryl, aryloxy, substituted aryloxy, cyano, halogen, hydroxyl, nitro, carboxyl, carboxyl esters, cycloalkyl, substituted cycloalkyl, heteroaryl, substituted heteroaryl, heterocyclic, and substituted heterocyclic.

“Amino” refers to the group —NH₂.

“Substituted amino” refers to the group —NR⁴¹R⁴¹, where each R⁴¹ group is independently selected from the group consisting of hydrogen, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO2-heteroaryl, —SO₂-substituted heteroaryl, —SO₂-heterocyclic, —SO₂-substituted heterocyclic, provided that both R⁴¹ groups are not hydrogen; or the R⁴¹ groups can be joined together with the nitrogen atom to form a heterocyclic or substituted heterocyclic ring.

“Acylamino” refers to the groups —NR⁴⁵C(O)alkyl, —NR⁴⁵C(O)substituted alkyl, —NR⁴⁵C(O)cycloalkyl, —NR⁴⁵C(O)substituted cycloalkyl, —NR⁴⁵C(O)alkenyl, —NR⁴⁵C(O)substituted alkenyl, —NR⁴⁵C(O)alkynyl, —NR⁴⁵C(O)substituted alkynyl, —NR⁴⁵C(O)aryl, —NR⁴⁵C(O)substituted aryl, —NR⁴⁵C(O)heteroaryl, —NR⁴⁵C(O)substituted heteroaryl, —NR⁴⁵C(O)heterocyclic, and —NR⁴⁵C(O)substituted heterocyclic where R⁴⁵ is hydrogen or alkyl and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are defined herein.

Carbonyloxyamino” refers to the groups —NR⁴⁶C(O)O-alkyl, —NR⁴⁶C(O)O-substituted alkyl, —NR⁴⁶C(O)O-alkenyl, —NR⁴⁶C(O)O-substituted alkenyl, —NR⁴⁶C(O)O-alkynyl, —NR⁴⁶C(O)O-substituted alkynyl, —NR⁴⁶C(O)O-cycloalkyl, —NR⁴⁶C(O)O-substituted cycloalkyl, —NR⁴⁶C(O)O-aryl, —NR⁴⁶C(O)O-substituted aryl, —NR⁴⁶C(O)O-heteroaryl, —NR⁴⁶C(O)O-substituted heteroaryl, —NR⁴⁶C(O)O-heterocyclic, and —NR⁴⁶C(O)O-substituted heterocyclic where R⁴⁶ is hydrogen or alkyl and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminocarbonyloxy” or as a prefix “carbamoyloxy” or “substituted carbamoyloxy” refers to the groups —OC(O)NR⁴⁷R⁴⁷ where each R⁴⁷ is independently hydrogen, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic or where each R⁴⁷ is joined to form, together with the nitrogen atom a heterocyclic or substituted heterocyclic and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminocarbonylamino” refers to the group —NR⁴⁹C(O)NR⁴⁹— R⁴⁹ is selected from the group consisting of hydrogen and alkyl.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is the aryl group. Preferred aryls include phenyl and naphthyl.

“Substituted aryl” refers to aryl groups, as defined herein, which are substituted with from 1 to 4, preferably 1-3, substituents selected from the group consisting of hydroxy, acyl, acylamino, carbonylaminothio, acyloxy, alkyl, substituted alkyl, alkoxy, substituted alkoxy, alkenyl, substituted alkenyl, alkynyl, substituted alkynyl, amidino, amino, substituted amino, aminoacyl, aminocarbonyloxy, aminocarbonylamino, aminothiocarbonylamino, aryl, substituted aryl, aryloxy, substituted aryloxy, cycloalkoxy, substituted cycloalkoxy, heteroaryloxy, substituted heteroaryloxy, heterocyclyloxy, substituted heterocyclyloxy, carboxyl, carboxyl esters cyano, thiol, alkylthio, substituted alkylthio, arylthio, substituted arylthio, heteroarylthio, substituted heteroarylthio, cycloalkylthio, substituted cycloalkylthio, heterocyclicthio, substituted heterocyclicthio, cycloalkyl, substituted cycloalkyl, guanidino, halo, nitro, heteroaryl, substituted heteroaryl, heterocyclic, substituted heterocyclic, oxycarbonylamino, oxythiocarbonylamino, —S(O)₂-alkyl, —S(O)₂-substituted alkyl, —S(O)₂-cycloalkyl, —S(O)₂-substituted cycloalkyl, —S(O)₂-alkenyl, —S(O)₂-substituted alkenyl, —S(O)₂-aryl, —S(O)₂-substituted aryl, —S(O)₂-heteroaryl, —S(O)₂-substituted heteroaryl, —S(O)₂-heterocyclic, —S(O)₂-substituted heterocyclic, —OS(O)₂-alkyl, —OS(O)₂-substituted alkyl, —OS(O)₂-aryl, —OS(O)₂-substituted aryl, —OS(O)₂-substituted heteroaryl, —OS(O)₂-heterocyclic, —OS(O)₂-substituted heterocyclic, —OSO₂—NR⁵¹R⁵¹ where each R⁵¹ is hydrogen or alkyl, —NR⁵¹S(O)₂-alkyl, —NR⁵¹S(O)₂NR⁵¹S(O)₂-aryl, —NR⁵¹S(O)₂-substituted aryl, —NR⁵¹S(O)₂-heteroaryl, —NR⁵¹S(O)₂-substituted heteroaryl, —NR⁵¹S(O)₂-heterocyclic, —NR⁵¹S(O)₂-substituted heterocyclic, —NR⁵¹S(O)₂—NR⁵¹-alkyl, —NR⁵¹S(O)₂—NR⁵¹-substituted alkyl, —NR⁵¹S(O)₂—NR⁵¹-aryl, —NR⁵¹S(O)₂—NR⁵¹-substituted aryl, —NR⁵¹S(O)₂—NR⁵¹-heteroaryl, —NR⁵¹S(O)₂—NR⁵¹-substituted heteroaryl, —NR⁵¹S(O)₂—NR⁵¹-heterocyclic, —NR⁵¹S(O)₂—NR⁵¹-substituted heterocyclic where each R⁵¹ is hydrogen or alkyl, wherein each of the terms is as defined herein.

“Aryloxy” refers to the group aryl-O— that includes, by way of example, phenoxy, naphthoxy, and the like.

“Substituted aryloxy” refers to substituted aryl-O— groups.

“Aryloxyaryl” refers to the group -aryl-O-aryl.

“Substituted aryloxyaryl” refers to aryloxyaryl groups substituted with from 1 to 3 substituents on either or both aryl rings as defined above for substituted aryl.

“Carboxyl” refers to —COOH or salts thereof.

“Carboxyl esters” refers to the groups —C(O)O-alkyl, —(O)O-substituted alkyl, —C(O)O-aryl, and —C(O)O-substituted aryl wherein alkyl, substituted alkyl, aryl and substituted aryl are as defined herein.

“Cycloalkyl” refers to cyclic alkyl groups of from 3 to 10 carbon atoms having single or multiple cyclic rings including, by way of example, adamantyl, cyclopropyl, cyclobutyl, cyclopentyl, cyclooctyl and the like.

“Substituted cycloalkyl” refers to a cycloalkyl group, having from 1 to 5 substituents selected from the group consisting of oxo (═O), thioxo (═S), alkoxy, substituted alkoxy, acyl, acylamino, acyloxy, amino, substituted amino, aminoacyl, aryl, substituted aryl, aryloxy, substituted aryloxy, cyano, halogen, hydroxyl, nitro, carboxyl, carboxyl esters, cycloalkyl, substituted cycloalkyl, heteroaryl, substituted heteroaryl, heterocyclic, and substituted heterocyclic.

“Cycloalkoxy” refers to —O-cycloalkyl groups.

“Substituted cycloalkoxy” refers to —O-substituted cycloalkyl groups.

“Halo” or “halogen” refers to fluoro, chloro, bromo and iodo and preferably is fluoro or chloro.

“Heteroaryl” refers to an aromatic group of from 1 to 15 carbon atoms, preferably from 1 to 10 carbon atoms, and 1 to 4 heteroatoms selected from the group consisting of oxygen, nitrogen and sulfur within the ring. Such heteroaryl groups can have a single ring (e.g., pyridinyl or furyl) or multiple condensed rings (e.g., indolizinyl or benzothienyl). Preferred heteroaryls include pyridinyl, pyrrolyl, indolyl, thiophenyl, and furyl.

“Substituted heteroaryl” refers to heteroaryl groups that are substituted with from 1 to 3 substituents selected from the same group of substituents defined for substituted aryl.

“Heteroaryloxy” refers to the group -0-heteroaryl and “substituted heteroaryloxy” refers to the group —O-substituted heteroaryl.

“Heterocycle” or “heterocyclic” refers to a saturated or unsaturated group having a single ring or multiple condensed rings, from 1 to 10 carbon atoms and from 1 to 4 hetero atoms selected from the group consisting of nitrogen, sulfur or oxygen within the ring wherein, in fused ring systems, one or more the rings can be aryl or heteroaryl provided that the point of attachment is at the heterocycle.

“Substituted heterocyclic” refers to heterocycle groups that are substituted with from 1 to 3 of the same substituents as defined for substituted cycloalkyl.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydro-isoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), piperidinyl, pyrrolidine, tetrahydrofuranyl, and the like.

“Heterocyclyloxy” refers to the group —O-heterocyclic and “substituted heterocyclyloxy” refers to the group -0-substituted heterocyclic.

“Thiol” or “mercapto” refers to the group —SH.

“Alkylsulfanyl” and “alkylthio” refer to the groups —S-alkyl where alkyl is as defined above.

“Substituted alkylthio” and “substituted alkylsulfanyl” refer to the group —S-substituted alkyl is as defined above.

“Cycloalkylthio” or “cycloalkylsulfanyl” refers to the groups —S-cycloalkyl where cycloalkyl is as defined above.

“Substituted cycloalkylthio” refers to the group —S-substituted cycloalkyl where substituted cycloalkyl is as defined above.

“Arylthio” refers to the group —S-aryl and “substituted arylthio” refers to the group —S-substituted aryl where aryl and substituted aryl are as defined above.

“Heteroarylthio” refers to the group —S-heteroaryl and “substituted heteroarylthio” refers to the group —S-substituted beteroaryl where heteroaryl and substituted heteroaryl are as defined above.

“Heterocyclicthio” refers to the group —S-heterocyclic and “substituted heterocyclicthio” refers to the group —S-substituted heterocyclic where heterocyclic and substituted heterocyclic are as defined above.

The term “amino acid” refers to any of the naturally occurring amino acids, as well as synthetic analogs (e.g., D-stereoisomers of the naturally occurring amino acids, such as D-threonine) and derivatives thereof. α-Amino acids comprise a carbon atom to which is bonded an amino group, a carboxyl group, a hydrogen atom, and a distinctive group referred to as a “side chain”. The side chains of naturally occurring amino acids are well known in the art and include, for example, hydrogen (e.g., as in glycine), alkyl (e.g., as in alanine, valine, leucine, isoleucine, proline), substituted alkyl (e.g., as in threonine, serine, methionine, cysteine, aspartic acid, asparagine, glutamic acid, glutamine, arginine, and lysine), arylalkyl (e.g., as in phenylalanine and tryptophan), substituted arylalkyl (e.g., as in tyrosine), and heteroarylalkyl (e.g., as in histidine). Unnatural amino acids are also known in the art, as set forth in, for example, Williams (ed.), Synthesis of Optically Active .alpha.-Amino Acids, Pergamon Press (1989); Evans et al., J. Amer. Chem. Soc., 112:40114030 (1990); Pu et al., J. Amer. Chem. Soc., 56:1280-1283 (1991); Williams et al., J. Amer. Chem. Soc., 113:9276-9286 (1991); and all references cited therein. The present invention includes the side chains of unnatural amino acids as well.

“Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “prodrug” refers to compounds of this invention which have been modified to include a physiologically and biocompatible removable group which group is removed in vivo to provide for the active drug, a pharmaceutically acceptable salt thereof or a biologically active metabolite thereof. Suitable removable groups are well known in the art and particularly preferred removable groups include esters of the carboxylic acid moiety on the glycine substituent. Preferably such esters include those derived from alkyl alcohols, substituted alkyl alcohols, hydroxy substituted aryls and heteroaryls and the like. Another preferred removable group are the amides formed from the carboxylic acid moiety on the glycine substituent. Suitable amides are derived from amines of the formula HNR²⁰R²¹ where R²⁰ and R²¹ are independently hydrogen, alkyl, substituted alkyl, aryl, substituted aryl, and the like.

It is understood that in all substituted groups defined above, polymers arrived at by defining substituents with further substituents to themselves (e.g., substituted aryl having a substituted aryl group as a substituent which is itself substituted with a substituted aryl group, etc) are not intended for inclusion herein. In such cases, the maximum number of such substituents is three. That is to say that each of the above definitions is constrained by a limitation that, for example, substituted aryl groups are limited to—substituted aryl-(substituted aryl)-substituted aryl.

Similarly, it is understood that the above definitions are not intended to include impermissible substitution patterns (e.g., methyl substituted with 5 fluoro groups or a hydroxyl group alpha to ethenylic or acetylenic unsaturation). Such impermissible substitution patterns are well known to the skilled artisan.

Diseases p The present invention provides an improved method of treating anemia.

The term “anemia” as used herein refers to any abnormality in hemoglobin or erythrocytes that leads to reduced oxygen levels in the blood. Anemia can be associated with abnormal production, processing, or performance of erythrocytes and/or hemoglobin. The term anemia refers to any reduction in the number of red blood cells and/or level of hemoglobin in blood relative to normal blood levels.

Anemia can arise due to conditions such as acute or chronic kidney disease, infections, inflammation, cancer, irradiation, toxins, diabetes, and surgery. Infections may be due to, e.g. virus, bacteria, and/or parasites, etc. Inflammation may be due to infection, autoimmune disorders, such as rheumatoid arthritis, etc. Anemia can also be associated with blood loss due to, e.g. stomach ulcer, duodenal ulcer, hemorrhoids, cancer of the stomach or large intestine, trauma, injury, surgical procedures, etc. Anemia is further associated with radiation therapy, chemotherapy, and kidney dialysis, e.g., chemotherapy-induced anemia, anemia associated with chronic kidney disease (CKD), etc. Anemia is also associated with HIV-infected patients undergoing treatment with azidothymidine (zidovudine) or other reverse transcriptase inhibitors, and can develop in cancer patients undergoing chemotherapy, e.g. with cyclic cisplatin-or non-cisplatin-containing chemotherapeutics. Aplastic anemia and myelodysplastic syndromes are diseases associated with bone marrow failure that result in decreased production of erythrocytes.

Further, anemia can result from defective or abnormal hemoglobin or erythrocytes, such as in disorders including microcytic anemia, hypochromic anemia, etc. Anemia can result from iron deficiency, either nutritionally based or related to disorders in iron uptake, mobilization, transport, processing, and utilization, see, e.g. sideroblastic anemia, etc.

The terms “disorders”, “diseases”, and “conditions” are used inclusively and refer to any condition deviating from normal.

The terms “anemic conditions” and “anemic disorders” refer to any condition, disease, or disorder associated with anemia. Such disorders include, but are not limited to, those disorders listed above. Anemic disorders further include, but are not limited to, aplastic anemia, autoimmune hemolytic anemia, bone marrow transplantation, Churg-Strauss syndrome, Diamond Blackfan anemia, Fanconi's anemia, Felty syndrome, graft versus host disease, hematopoietic stem cell transplantation, hemolytic uremic syndrome, myelodysplastic syndrome, nocturnal paroxysmal hemoglobinuria, osteomyelofibrosis, pancytopenia, pure red-cell aplasia, purpura Schoenlein-Henoch, sideroblastic anemia, refractory anemia with excess of blasts, rheumatoid arthritis, Shwachman syndrome, sickle cell disease, thalassemia major, thalassemia minor, thrombocytopenic purpura, etc.

Subjects

The present invention relates to the administration of an effective amount of a compound of the invention to a subject having anemia.

The invention is applicable to a variety of different organisms, including for example, vertebrates, large animals and primates. In a preferred embodiment, the subject is a mammalian subject, and in a most preferred embodiment, the subject is a human subject. However, although medical applications with humans are clearly foreseen, veterinary applications are also envisaged here.

The methods of the present invention are particularly suitable for subjects who are resistant or hyporesponsive to rhEPO treatment. Such subjects are often administered higher doses of rhEPO and are therefore also more likely to suffer from the associated complications and risks associated with rhEPO treatment. Human subjects that are hyporesponsive to rhEPO treatment, including subjects that may show an increased risk of morbidity and mortality, may be identified using the definition provided in Zhang et al., (2004) Am J Kidney Disease 44:866-876. Here, hyporesponsiveness is defined as a consistent difficulty in increasing hematocrit levels to greater than 33% or the requirement for high rhEPO doses. Another suitable definition is given in Raffaele et al. (2001) Dialysis and Transplantation 30(6): 368-372, wherein a need for a patient to receive greater than 300 IU/kg/wk rhEPO to obtain a desired response was used to define that patient as resistant.

Accordingly, in preferred embodiments of the present invention, the subject has previously been treated with rhEPO therapy. For example, the subject may have been treated with rhEPO therapy within the last 10 years, 5 years, 4 years, 3 years, 2 years, or 1 year. In particular, the subject may have been treated with rhEPO therapy within the last 6 months, 5 months, 4 months, 3 months, 2 months, or 1 month. In some embodiments, the rhEPO therapy has ceased before the subject is treated with the methods of the present invention. For example, the rhEPO therapy may have ceased 10 years, 5 years, 4 years, 3 years, 2 years, or 1 year before treatment with the methods of the present invention. In particular, the rhEPO therapy may have ceased 6 months, 5 months, 4 months, 3 months, 2 months, or 1 month before treatment with the methods of the present invention. However, the interval between rhEPO therapy and the methods of the present invention may be shorter than this, for example 30 days, 21 days, 14 days, 10 days, 7 days, 4 days, 3 days, 2 days, or 1 day. In preferred embodiments, the rhEPO therapy will have been stopped because of increased risk of associated complications, e.g. thrombotic complications, in the subject.

It is also possible for the subject of the present invention to continue to undergo rhEPO therapy in combination with the methods of the present invention. Thus, the subject may be undergoing treatment with rhEPO therapy (this treatment not having been ceased). The methods of the present invention may therefore be used in conjunction with rhEPO therapy and other ESP therapy. For example, in some embodiments of the present invention, the subject is administered a compound of the present invention in simultaneous, separate, or sequential administration with rhEPO. In such embodiments, the subject may be administered with a lower dose of rhEPO than when rhEPO is administered as a single therapy.

The side effects of rhEPO therapy are particularly seen in its use in the treatment of chemotherapy-induced anemia, i.e. in the treatment of anemia in cancer patients who are undergoing chemotherapy. Accordingly, the methods of the present invention are particularly envisaged for the treatment of subjects with chemotherapy-induced anemia. In such embodiments, the compounds of the present invention may be used in combination with the relevant agent used in the chemotherapy. Thus, the compounds of the present invention may be used in simultaneous, separate, or sequential administration with the chemotherapy agent. Relevant chemotherapy agents for use in this embodiment of the invention are well known to those of skill in the art and include, but are not limited to, the main classes of chemotherapy agents, i.e. alkylating agents (e.g. busulfan, cisplatin, carboplatin, chlorambucil, cyclophosphamide, ifosfamide, dacarbazine, mechlorethamine, melphalan and temozolomide); nitrosoureas (e.g. carmustine and lomustine); antimetabolites (5-fluorouracil, capecitabine, 6-mercaptopurine, methotrexate, gemcitabine, cytarabine, fludarabine and pemetrexed); anthracyclines and related drugs (e.g. daunorubicin, doxorubicin, epirubicin, idarubicin and mitoxantrone); topoisomerase II inhibitors (e.g. topotecan, irinotecan, etoposide and teniposide); mitotic inhibitors (e.g. taxanes (paclitaxel, docetaxel) and the vinca alkaloids (vinblastine, vincristine and vinorelbine)); and corticosteroid hormones (e.g. prednisone and dexamethasone).

The likelihood of complications arising from rhEPO therapy is also greater in subjects who have a history of thrombosis. Accordingly, the methods of the present invention are particularly suited to subjects who have a history of thrombosis or thrombotic complications. Within this context, subjects with a history of thrombosis include, but are not limited to, those who have a family history of thrombotic events or who have experienced thrombotic events in the last 20 years, 10 years, 5 years, 4 years, 3 years, 2 years, or 1 year. Thrombotic events are well known to those in the art and include, but are not limited to, venous thrombosis (e.g. deep vein thrombosis, retinal vein thrombosis etc.); arterial thrombosis (e.g. myocardial infarction, cerebrovascular accident etc.) and embolism (e.g. pulmonary embolism etc.). Subjects with a history of such events are particularly envisaged in the present invention.

Similarly, in other preferred embodiments, the subjects of the present invention are those with risk factors for developing thrombosis. One such risk factor is a history of thrombosis, as discussed above. However, numerous other risk factors will be known to those of skill in the art and include, but are not limited to, increasing age, male gender, exposure to tobacco smoke, high blood cholesterol levels, high blood pressure, obesity, diabetes mellitus, physical inactivity, and stress. Subjects demonstrating one or more or these risk factors are particularly envisaged in the present invention.

Many therapeutic strategies are available for reducing thrombosis, and the methods of the present invention can be envisaged in combination with such therapies. This is particularly the case when the methods of the invention are combined with rhEPO therapy as described supra. Suitable agents for reducing thrombosis for use in this embodiment of the invention are well known to those of skill in the art and include, but are not limited to, the administration of aspirin, warfarin (particularly in combination with aspirin), beta-blockers, calcium-channel blockers, ACE inhibitors, nitrates, and statins. Accordingly, in some embodiments of the invention, the compound of the invention is for administration simultaneous, separate, or sequential administration with such an agent.

Subjects suitable for treatment using the methods of the present invention include subjects having hemoglobin levels below normal levels, e.g., human adult male subjects having hemoglobin levels below 14 gm/dL, human adult female subjects having hemoglobin levels below 13.7 gm/dL, etc. In particular embodiments, the subjects suitable for treatment with the methods of the present invention are subjects having hemoglobin levels below normal levels, such as human adults having hemoglobin levels below 13 gm/dL, below 12 gm/dL, below 11 gm/dL, and below 10 gm/dL.

Additional subjects suitable for treatment using the methods of the present invention include subjects having hematocrit below normal levels; for example, human adult male subjects having hematocrit below 42%. In particular embodiments, the subject suitable for treatment with the methods of the present invention are subjects having hematocrit below normal levels, such as human adults having hematocrit below 39%, below 36%, below 33%, and below 30%.

Preferably, administration of an agent of the present invention to a subject results in an increase in baseline hemoglobin level in that subject by a level in the range of 0.1-5.0 g/dL. In some embodiments, the level is increased by a level in the range of 0.2-5.0 g/dL, 0.5-5.0 g/dL, 1.0-5.0 g/dL, 1.5-5.0 g/dL, 2.0-5.0 g/dL, 3.0-5.0 g/dL, or 4.0-5.0 g/dL. More preferably, it is raised to a level in the range 0.2-2.5 g/dL, 0.4-2.5 g/dL, 0.6-2.5 g/dL, 0.8-2.5 g/dL, 1.0-2.5 g/dL, 1.2-2.5 g/dL, 1.4-2.5 g/dL, 1.6-2.5 g/dL, 1.8-2.5 g/dL, or 2-2.5 g/dL. More preferably still, it is raised to a level in the range of 1.0-2.0 g/dL, 1.1-2.0 g/dL, 1.2-2.0 g/dL, 1.3-2.0 g/dL, 1.4-2.0 g/dL, 1.5-2.0 g/dL, 1.6-2.0 g/dL, 1.7-2.0 g/dL, 1.8-2.0 g/dL, or 1.9-2.0 g/dL.

Preferably, administration of an agent of the present invention to a subject results in an increase in the circulating level of EPO in that subject to a level in the range of 10-1000 mIU/ml (assuming a basal endogenous level of 10 mIU/ml). In some embodiments, the level is raised to a level in the range of 10-500 mIU/ml, 10400 mIU/ml, 10-300 mIU/ml, 10-200 mIU/ml, 10-150 mIU/ml, 10-100 mIU/ml, 10-90 mIU/ml, 10-80 mIU/ml, 10-70 mIU/ml, 10-60 mIU/ml, 10-50 mIU/ml, 1040 mIU/ml, 10-30 mIU/ml, 10-20 mIU/ml, or 10-15 mIU/ml. More preferably, it is raised to a level in the range of 10-100 mIU/ml, 10-75 mIU/ml, 10-50 mIU/ml, 10-25 mIU/ml, or 10-15 mIU/ml. More preferably still, it is raised to a level in the range of only 10-50 mIU/ml, 1045 mIU/ml, 1040 mIU/ml, 10-35 mIU/ml, 10-30 mIU/ml, 10-25 mIU/ml, 10-20 mIU/ml, or 10-15 mIU/ml.

Subjects that are particularly suitable for treatment according to the methods of the invention are those that are refractory to rhEPO treatment. Such a subject may generally be characterised by requiring high levels of rhEPO administration to achieve hemoglobin levels that have a positive effect on their disease condition. For example, it has been found that administration of equivalent doses of rhEPO to achieve a similar increase in hemoglobin in the subject to that achieved using a method according to the present invention results in a greater increase in the circulating level of EPO, for example to a level in the range of 100 to 20 000 mIU/ml. These levels of EPO are disadvantageous, as described in more detail throughout the present specification.

Expressed more simply, the methods of the invention achieve physiologically beneficial levels of hemoglobin whilst simultaneously raising EPO levels by only a fraction of the levels necessary to achieve the same levels using rhEPO. This fraction may be less than 50%, more preferably less than 40%, more preferably less than 30%, more preferably less than 20%, more preferably less than 15%, more preferably less than 10%, even more preferably less than 5%, more preferably even less than 1%.

Modes of Administration

The compositions of the present invention can be delivered directly or in pharmaceutical compositions contaming excipients, as is well known in the art. The present methods of treatment involve administration of an effective amount of a compound of the present invention to a subject having anemia.

An effective amount, e.g., dose, of compound or drug can readily be determined by routine experimentation, as can an effective and convenient route of administration and an appropriate formulation. Various formulations and drug delivery systems are available in the art. (See, e.g., Gennaro, ed. (2000) Remington's Pharmaceutical Sciences, supra; and Hardman, Limbird, and Gilman, eds. (2001) The Pharmacological Basis of Therapeutics, supra.)

Suitable routes of administration may, for example, include oral, rectal, topical, nasal, pulmonary, ocular, intestinal, and parenteral administration. Primary routes for parenteral administration include intravenous, intramuscular, and subcutaneous administration. Secondary routes of administration include intraperitoneal, intra-arterial, intra-articular, intracardiac, intracisternal, intradermal, intralesional,.intraocular, intrapleural, intrathecal, intrauterine, and intraventricular administration. The indication to be treated, along with the physical, chemical, and biological properties of the drug, dictate the type of formulation and the route of administration to be used, as well as whether local or systemic delivery would be preferred.

In preferred embodiments, the compounds of the present invention are administered orally. Oral administration is particularly preferred for the preferred compounds of the invention (e.g. [(1-Chloro-4-hydroxy-isoquinoline-3-carbonyl)-amino]-acetic acid (Compound A), [(4-Hydroxy-1-methyl-7-phenoxy-isoquinoline-3-carbonyl)-amino]-acetic acid (Compound B), {[4-Hydroxy-7-(4-methoxy-phenoxy)-isoquinoline-3-carbonyl]-amino}-acetic acid (Compound C). [(4-Hydroxy-8-phenoxy-isoquinoline-3-carbonyl)-amino]-acetic acid (Compound D), [(4-Hydroxy-1-methyl-8-phenoxy-isoquinoline-3-carbonyl)-amino]-acetic acid (Compound E), [(7-Chloro-4-hydroxy-1-methyl-isoquinoline-3-carbonyl)-amino]-acetic acid (Compound F), {[8-(4-Fluoro-phenoxy)-4-hydroxy-1-methyl-isoquinoline-3-carbonyl]-amino}-acetic acid (Compound G), and [(4-cyano-7-hydroxy-thieno[3,2-c]pyridine-6-carbonyl)-amino]-acetic acid (Compound H )).

Pharmaceutical dosage forms of a compound of the invention may be provided in an instant release, controlled release, sustained release, or target drug-delivery system. Commonly used dosage forms include, for example, solutions and suspensions, (micro-) emulsions, ointments, gels and patches, liposomes, tablets, dragees, soft or hard shell capsules, suppositories, ovules, implants, amorphous or crystalline powders, aerosols, and lyophilized formulations. Depending on route of administration used, special devices may be required for application or administration of the drug, such as, for example, syringes and needles, inhalers, pumps, injection pens, applicators, or special flasks. Pharmaceutical dosage forms are often composed of the drug, an excipient(s), and a container/closure system. One or multiple excipients, also referred to as inactive ingredients, can be added to a compound of the invention to improve or facilitate manufacturing, stability, administration, and safety of the drug, and can provide a means to achieve a desired drug release profile. Therefore, the type of excipient(s) to be added to the drug can depend on various factors, such as, for example, the physical and chemical properties of the drug, the route of administration, and the manufacturing procedure. Pharmaceutically acceptable excipients are available in the art, and include those listed in various pharmacopoeias. (See, e.g., USP, JP, EP, and BP, FDA web page (www.fda.gov), Inactive Ingredient Guide 1996, and Handbook of Pharmaceutical Additives, ed. Ash; Synapse Information Resources, Inc. 2002.)

Pharmaceutical dosage forms of a compound of the present invention may be manufactured by any of the methods well-known in the art, such as, for example, by conventional mixing, sieving, dissolving, melting, granulating, dragee-making, tabletting, suspending, extruding, spray-drying, levigating, emulsifying, (nano/micro-) encapsulating, entrapping, or lyophilization processes. As noted above, the compositions of the present invention can include one or more physiologically acceptable inactive ingredients that facilitate processing of active molecules into preparations for pharmaceutical use.

Proper formulation is dependent upon the desired route of administration. For intravenous injection, for example, the composition may be formulated in aqueous solution, if necessary using physiologically compatible buffers, including, for example, phosphate, histidine, or citrate for adjustment of the formulation pH, and a tonicity agent, such as, for example, sodium chloride or dextrose. For transmucosal or nasal administration, semisolid, liquid formulations, or patches may be preferred, possibly containing penetration enhancers. Such penetrants are generally known in the art. For oral administration, the compounds can be formulated in liquid or solid dosage forms and as instant or controlled/sustained release formulations. Suitable dosage forms for oral ingestion by a subject include tablets, pills, dragees, hard and soft shell capsules, liquids, gels, syrups, slurries, suspensions, and emulsions. The compounds may also be formulated in rectal compositions, such as suppositories or retention enemas, e.g., containing conventional suppository bases such as cocoa butter or other glycerides.

Solid oral dosage forms can be obtained using excipients, which may include, fillers, disintegrants, binders (dry and wet), dissolution retardants, lubricants, glidants, antiadherants, cationic exchange resins, wetting agents, antioxidants, preservatives, coloring, and flavoring agents. These excipients can be of synthetic or natural source. Examples of such excipients include cellulose derivatives, citric acid, dicalcium phosphate, gelatine, magnesium carbonate, magnesium/sodium lauryl sulfate, mannitol, polyethylene glycol, polyvinyl pyrrolidone, silicates, silicium dioxide, sodium benzoate, sorbitol, starches, stearic acid or a salt thereof, sugars (i.e. dextrose, sucrose, lactose, etc.), talc, tragacanth mucilage, vegetable oils (hydrogenated), and waxes. Ethanol and water may serve as granulation aides. In certain instances, coating of tablets with, for example, a taste-masking film, a stomach acid resistant film, or a release-retarding film is desirable. Natural and synthetic polymers, in combination with colorants, sugars, and organic solvents or water, are often used to coat tablets, resulting in dragees. When a capsule is preferred over a tablet, the drug powder, suspension, or solution thereof can be delivered in a compatible hard or soft shell capsule.

In one embodiment, the compounds of the present invention can be administered topically, such as through a skin patch, a semi-solid or a liquid formulation, for example a gel, a (micro)-emulsion, an ointment, a solution, a (nano/micro)-suspension, or a foam. The penetration of the drug into the skin and underlying tissues can be regulated, for example, using penetration enhancers; the appropriate choice and combination of lipophilic, hydrophilic, and amphiphilic excipients, including water, organic solvents, waxes, oils, synthetic and natural polymers, surfactants, emulsifiers; by pH adjustment; and use of complexing agents. Other techniques, such as iontophoresis, may be used to regulate skin penetration of a compound of the invention. Transdermal or topical administration would be preferred, for example, in situations in which local delivery with minimal systemic exposure is desired.

For administration by inhalation, or administration to the nose, the compounds for use according to the present invention are conveniently delivered in the form of a solution, suspension, emulsion, or semisolid aerosol from pressurized packs, or a nebuliser, usually with the use of a propellant, e.g., halogenated carbons dervided from methan and ethan, carbon dioxide, or any other suitable gas. For topical aerosols, hydrocarbons like butane, isobutene, and pentane are useful. In the case of a pressurized aerosol, the appropriate dosage unit may be determined by providing a valve to deliver a metered amount. Capsules and cartridges of, for example, gelatin, for use in an inhaler or insufflator, may be formulated. These typically contain a powder mix of the compound and a suitable powder base such as lactose or starch.

Compositions formulated for parenteral administration by injection are usually sterile and, can be presented in unit dosage forms, e.g., in ampoules, syringes, injection pens, or in multi-dose containers, the latter usually containing a preservative. The compositions may take such forms as suspensions, solutions, or emulsions in oily or aqueous vehicles, and may contain formulatory agents, such as buffers, tonicity agents, viscosity enhancing agents, surfactants, suspending and dispersing agents, antioxidants, biocompatible polymers, chelating agents, and preservatives. Depending on the injection site, the vehicle may contain water, a synthetic or vegetable oil, and/or organic co-solvents. In certain instances, such as with a lyophilized product or a concentrate, the parenteral formulation would be reconstituted or diluted prior to administration. Depot formulations, providing controlled or sustained release of a compound of the invention, may include injectable suspensions of nano/micro particles or nano/micro or non-micronized crystals. Polymers such as poly(lactic acid), poly(glycolic acid), or copolymers thereof, can serve as controlled/sustained release matrices, in addition to others well known in the art. Other depot delivery systems may be presented in form of implants and pumps requiring incision.

Suitable carriers for intravenous injection for the molecules of the invention are well-known in the art and include water-based solutions containing a base, such as, for example, sodium hydroxide, to form an ionized compound, sucrose or sodium chloride as a tonicity agent, for example, the buffer contains phosphate or histidine. Co-solvents, such as, for example, polyethylene glycols, may be added. These water-based systems are effective at dissolving compounds of the invention and produce low toxicity upon systemic administration. The proportions of the components of a solution system may be varied considerably, without destroying solubility and toxicity characteristics. Furthermore, the identity of the components may be varied. For example, low-toxicity surfactants, such as polysorbates or poloxamers, may be used, as can polyethylene glycol or other co-solvents, biocompatible polymers such as polyvinyl pyrrolidone may be added, and other sugars and polyols may substitute for dextrose.

For composition useful for the present methods of treatment, a therapeutically effective dose can be estimated initially using a variety of techniques well-known in the art. Initial doses used in animal studies may be based on effective concentrations established in cell culture assays. Dosage ranges appropriate for human subjects can be determined, for example, using data obtained from animal studies and cell culture assays.

A therapeutically effective dose or amount of a compound, agent, or drug of the present invention refers to an amount or dose of the compound, agent, or drug that results in amelioration of symptoms or a prolongation of survival in a subject. Toxicity and therapeutic efficacy of such molecules can be determined by standard pharmaceutical procedures in cell cultures or experimental animals, e.g., by determining the LD50 (the dose lethal to 50% of the population) and the ED50 (the dose therapeutically effective in 50% of the population). The dose ratio of toxic to therapeutic effects is the therapeutic index, which can be expressed as the ratio LD50/ ED50. Agents that exhibit high therapeutic indices are preferred.

The effective amount or therapeutically effective amount is the amount of the compound or pharmaceutical composition that will elicit the biological or medical response of a tissue, system, animal, or human that is being sought by the researcher, veterinarian, medical doctor, or other clinician, e.g., an increase in hemoglobin levels, an increase in hematocrit, treatment of anemia, an increase in quality of life, etc.

Dosages preferably fall within a range of circulating concentrations that includes the ED50 with little or no toxicity. Dosages may vary within this range depending upon the dosage form employed and/or the route of administration utilized. The exact formulation, route of administration, dosage, and dosage interval should be chosen according to methods known in the art, in view of the specifics of a subject's condition.

Dosage amount and interval may be adjusted individually to provide plasma levels of the active moiety that are sufficient to achieve the desired effects, i.e., minimal effective concentration (MEC). The MEC will vary for each compound but can be estimated from, for example, in vitro data and animal experiments. Dosages necessary to achieve the MEC will depend on individual characteristics and route of administration. In cases of local administration or selective uptake, the effective local concentration of the drug may not be related to plasma concentration.

In some embodiment of the present invention, effective doses for preferred compounds of the invention (e.g. [(1-Chloro-4-hydroxy-isoquinoline-3-carbonyl)-amino]-acetic acid (Compound A), [(4-Hydroxy-1-methyl-7-phenoxy-isoquinoline-3-carbonyl)-amino]-acetic acid (Compound B), {[4-Hydroxy-7-(4-methoxy-phenoxy)-isoquinoline-3-carbonyl]-amino}-acetic acid (Compound C). [(4-Hydroxy-8-phenoxy-isoquinoline-3-carbonyl)-amino]-acetic acid (Compound D), [(4-Hydroxy-1-methyl-8-phenoxy-isoquinoline-3-carbonyl)-amino]-acetic acid (Compound E), [(7-Chloro-4-hydroxy-1-methyl-isoquinoline-3-carbonyl)-amino]-acetic acid (Compound F), {[8-(4-Fluoro-phenoxy)-4-hydroxy-1-methyl-isoquinoline-3-carbonyl]-amino}-acetic acid (Compound G), and [(4-cyano-7-hydroxy-thieno[3,2-c]pyridine-6-carbonyl)-amino]-acetic acid (Compound H ) include 3 mg/kg, 6 mg/kg, 10 mg/kg, 15 mg/kg, 20 mg/kg and 30 mg/kg. These doses are therefore particularly preferred for use in the present invention.

In additional embodiments, effective treatment regimes for preferred compounds of the invention (e.g. [(1-Chloro-4-hydroxy-isoquinoline-3-carbonyl)-amino]-acetic acid (Compound A), [(4-Hydroxy-1-methyl-7-phenoxy-isoquinoline-3-carbonyl)-amino]-acetic acid (Compound B), {[4-Hydroxy-7-(4-methoxy-phenoxy)-isoquinoline-3-carbonyl]-amino}-acetic acid (Compound C). [(4-Hydroxy-8-phenoxy-isoquinoline-3-carbonyl)-amino]-acetic acid (Compound D), [(4-Hydroxy-1-methyl-8-phenoxy-isoquinoline-3-carbonyl)-amino]-acetic acid (Compound E), [(7-Chloro-4-hydroxy-1-methyl-isoquinoline-3-carbonyl)-amino]-acetic acid (Compound F), {[8-(4-Fluoro-phenoxy)-4-hydroxy-1-methyl-isoquinoline-3-carbonyl]-amino}-acetic acid (Compound G), and [(4-cyano-7-hydroxy-thieno[3,2-c]pyridine-6-carbonyl)-amino]-acetic acid (Compound H )) include administration two or three times weekly. These regimes are therefore particularly preferred for use in the present invention.

The invention contemplates in various aspects that the present methods of treatment can be administered in conjunction with additional therapies, including, for example, ESP therapies, such as rhEPO therapy. In certain aspects, this involves administration of rhEPO or other ESPs at levels sufficiently low to minimize or remove the risk of thrombosis or thrombotic complications, the inconvenience to the subject, and the other risks and costs associated with standard rhEPO and ESP therapy. In other aspects, the present methods are applied in conjunction with other methods of therapy, such as rhEPO or ESP therapy, wherein the rhEPO or other ESPs are administered at levels sufficiently low to minimize or remove the risk of iron overload and to minimize the increased cost and inconvenience to subject that is associated with standard rhEPO and ESP therapy. Finally, in further aspects, the present methods are applied in conjunction with other methods of therapy, such as anti-tumor necrosis factor (TNF) therapy, wherein the anti-TNF agents are administered at levels sufficiently low to minimize or remove associated risks and costs.

The amount of agent or composition administered may be dependent on a variety of factors, including the sex, age, and weight of the subject being treated, the severity of the affliction, the manner of administration, and the judgment of the prescribing physician.

The present compositions may, if desired, be presented in a pack or dispenser device containing one or more unit dosage forms containing the active ingredient. Such a pack or device may, for example, comprise metal or plastic foil, such as a blister pack, or glass and rubber stoppers such as in vials. The pack or dispenser device may be accompanied by instructions for administration. Compositions comprising a compound of the invention formulated in a compatible pharmaceutical carrier may also be prepared, placed in an appropriate container, and labeled for treatment of an indicated condition.

These and other embodiments of the present invention will readily occur to those of ordinary skill in the art in view of the disclosure herein.

### EXAMPLES

The invention will be further understood by reference to the following examples, which are intended to be purely exemplary of the invention. These examples are provided solely to illustrate the claimed invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description and accompanying figures. Such modifications are intended to fall within the scope of the appended claims.

### Example 1

### The Present Methods and Compounds Increase Circulating EPO Levels in Mice In Vivo

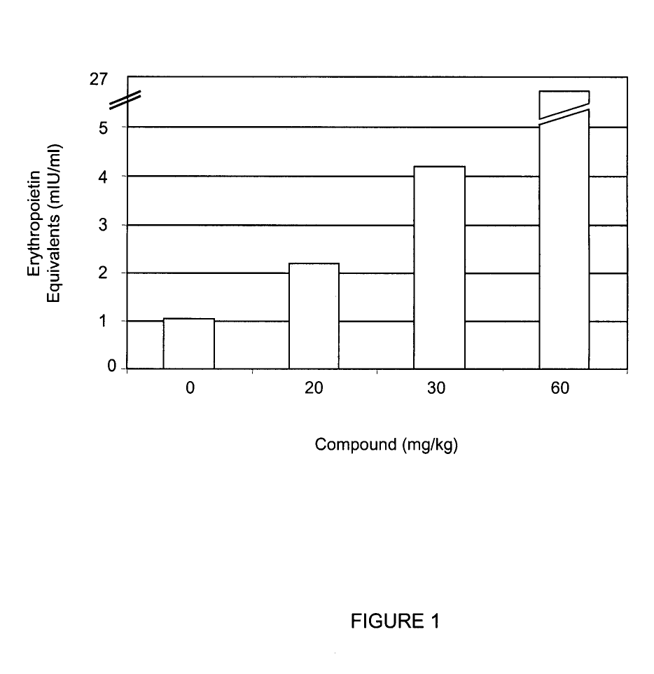

Mice were administered various doses (0, 20, 30, 60 mg/kg) of compound A by oral gavage. Circulating levels of EPO were determined 6 hours after compound administration. As shown in FIG. 1, administration of compound A increased EPO levels in mice in a dose-dependent manner. Administration of 20 mg/kg of compound A increased circulating levels of EPO approximately two-fold.

### Example 2

### The Present Methods and Compounds Increase Circulating EPO Levels in Rats In Vivo

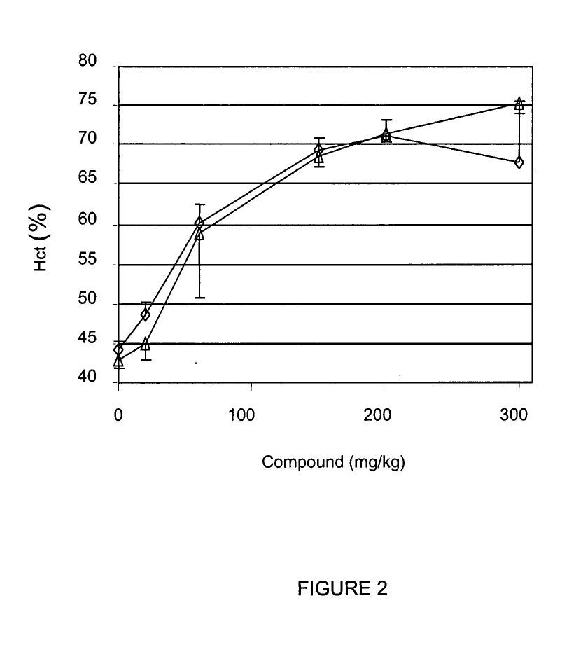

Male (diamonds in FIG. 2) and female (triangles in FIG. 2) rats were administered various doses (20, 60, 150, 200, 300 mg/kg) of compound A two times per week (e.g., intermittent dosing) for 4 weeks. Hematocrit was determined on day 32. As shown in FIG. 2, administration of compound A increased hematocrit in rats in a dose-dependent manner. These results showed that a clinically-significant increase in erythropoiesis, as determined by hematocrit, occurred at 20 mg/kg dose administration.

### Example 3

### The Present Methods and Compounds Increase Circulating EPO Levels in Healthy Human Subjects

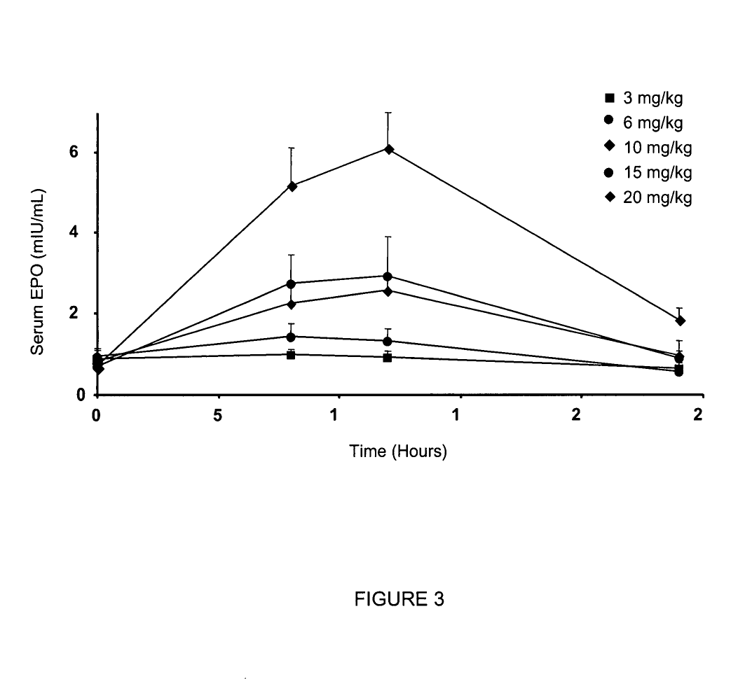

Healthy human subject volunteers were administered various concentrations (3, 6, 10, 15, 20 mg/kg) of compound A by oral gavage. At the indicated times (hours) after compound administration, serum EPO levels were determined. As shown in FIG. 3, administration of compound A increased serum EPO levels in a dose-dependent manner in healthy human subjects. These results showed that methods and compounds of the present invention are useful for inducing endogenous EPO levels.

Increases in circulating EPO levels following administration of compound A or of rhEPO were compared. Table 3 and Table 2 below show Cmax EPO and EPO Area Under the Curve (AUC) values following intravenous (i.v.) or subcutaneous (s.c.) administration of various doses of rhEPO to human subjects, respectively. As shown in Table 3 and Table 2, administration of rhEPO resulted in high concentrations of circulating EPO levels (Cmax EPO) and high circulating EPO levels over a period of time (EPO AUC) following rhEPO administration. By contrast, administration of various therapeutically effective (e.g., effective at increaseing Hb or Hct) doses of compound A resulted in substantially lower circulating EPO levels than those observed following rhEPO administration. (See Table 1 below.)

TABLE 1

| PHI Dose | Cmax EPO¹ (12 hours) | EPO AUC (0-24 hours) |
|---|---|---|
| 6 mg/kg | 14.2 | 203.5 |
| 10 mg/kg | 26.3 | 331 |
| 20 mg/kg | 51.2 | 365.5 |
| 30 mg/kg | 73 | 508 |
| 90 minutes hypoxia² | 14 | ND |

TABLE 2

| rhEPO U/kg (i.v.) | Cmax EPO¹ | EPO AUC (0-48 hours) |
|---|---|---|
| 10 | 181 | 731 |
| 50 | 1,430 | 9,306 |
| 150 | 4,438 | 30,990 |
| 500 | 16,257 | 142,480 |

TABLE 3

| RhEPO U/kg (s.c.) | Cmax EPO¹ | EPO AUC (0-672 hours) |
|---|---|---|
| 300 | 429 | 20,056 |
| 450 | 1,263 | 45,498 |
| 600 | 1,263 | 55,475 |

¹mIU/ml; assumes basal endogenous EPO of ˜10 mIU/ml
²Simulated altitude of 5,000 meters

Taken together, the data shown in Tables 1, 2, and 3 indicated that compound A is approximately 50- to 100-fold more potent (based on comparison of Cmax values) for obtaining a therapeutically effective amount of circulating EPO. Additionally, these data also showed that compound A is approximately 20- to 50-fold more potent (based on comparison of EPO AUC) for a therapeutically effective amount of circulating EPO. Therefore, in some embodiments, the therapeutic agents used in the present methods can be administered and can be therapeutically effective in amounts one-tenth to one-twentieth, for example, of the levels at which rhEPO and other ESPs would be administered to achieve similar therapeutic effect.

### Example 4

### The Present Methods and Compounds Increase Circulating EPO Levels in Monkeys In Vivo

Table 4 shows peak circulating (i.e., serum) EPO levels in normal monkeys administered (single dose/monkey) various doses (3, 13, 30, 40, 50, 60 mg/kg) of compound A or of compound B. Eight to twelve hours after compound administration, circulating EPO levels were determined.

TABLE 4

| Dose | Compound A | Compound A | Compound B | Compound B |
|---|---|---|---|---|
| (mg/kg) | Pre-dose | Peak EPO | Pre-dose | Peak EPO |
| 3 | ND | ND | 4.2 | 13.1 |
| 13 | 2.1 | 3.6 | ND | ND |
| 30 | 0.6 | 2.9 | 0 | 1534.0 |
| 40 | 0.8 | 32.3 | ND | ND |
| 50 | 0 | 21.4 | ND | ND |
| 60 | 0 | 1194.2 | 1.2 | 2739.8 |

### Example 5

### The Present Methods and Compounds Increase Circulating EPO Levels in Bilateral Nephrectomized-Mice In Vivo

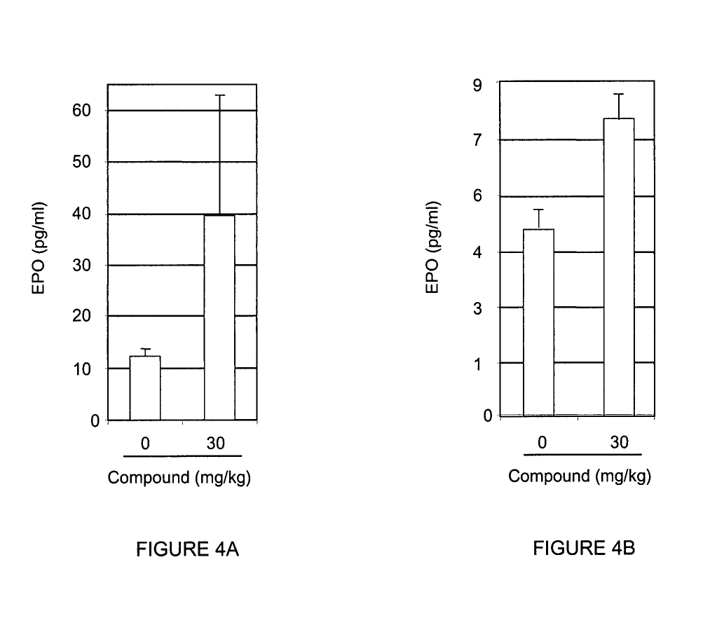

Mice underwent bilateral nephrectomy (BN) surgery (or were sham-operated). Two hours after BN surgery, mice were administered a single oral dose (30 mg/kg) of compound A. Circulating EPO levels were measured 6 hours after compound administration. As shown in FIG. 4A (sham) and FIG. 4B (BN), significant increase in EPO levels was observed in BN mice treated with compound A. Sham-operated mice treated with compound A also showed an increase in circulating levels of EPO. The kidneys produce the majority of endogenous EPO. These results indicated that compounds of the present invention are able to increase EPO levels from non-renal sources. These results suggested that methods and compounds of the present invention are efficacious in treating anemia in patients with reduced renal mass or reduced renal function, such as, for example, patients with chronic kidney disease or end-stage renal disease.

### Example 6

### The Present Methods and Compounds were Therapeutically Effective in Treating Anemia in Human Subjects with CKD

The effects of compound of the present invention on erythropoiesis in anemic pre-dialysis subjects with advanced stage chronic kidney disease were determined. Study subjects had chronic kidney disease and anemia, having GFR<30 ml/min and Hb<10g/dL. Two anemic pre-dialysis subject populations with chronic kidney disease (CKD) were studied: (1) subjects with no previous exposure to rhEPO (i.e., rhEPO-naive) and (2) subjects who had been receiving continuous rhEPO therapy for at least 8 weeks. In rhEPO-treated subjects, rhEPO administration was discontinued 5-14 days prior to initiation of treatment with compound of the present invention Subjects were orally administered compound A three-times per week for 4 weeks. Erythropoiesis was measured by changes in hemoglobin levels and serum EPO concentrations.

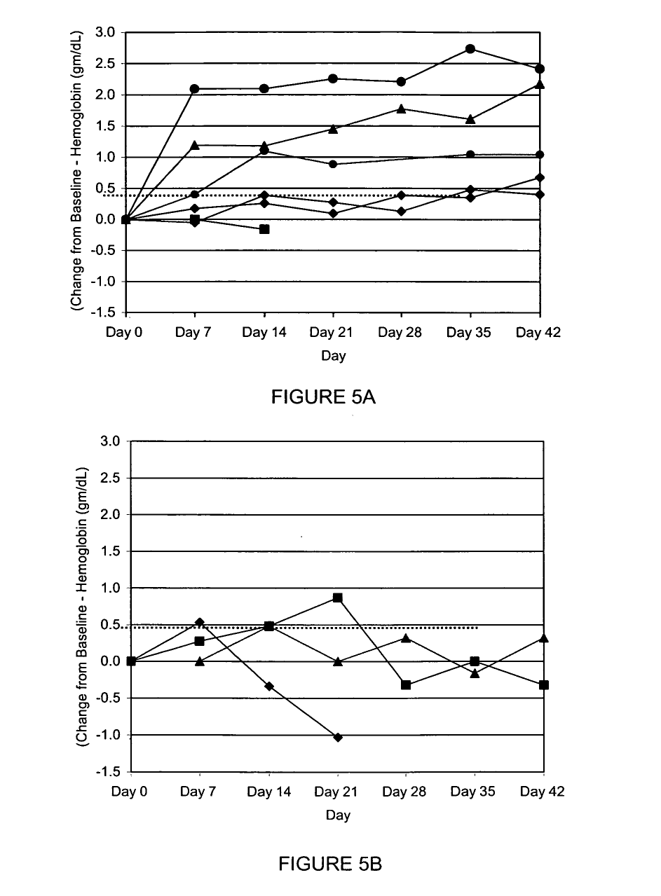

As shown in FIG. 5, hemoglobin levels were higher in rhEPO-naive subjects treated with compound A (FIG. 5A) than in rhEPO-naive placebo-treated subjects (FIG. 5B). As shown in Table 5 below, subjects administered compound A showed a mean increase in Hb of 1.9 g/dL from baseline levels; subjects administered placebo showed a mean decrease in Hb of 0.35 g/dL from baseline levels.

TABLE 5

| | | Mean change from Baseline |
|---|---|---|
| | Mean Baseline | Hb (g/dL) Day 42* |
| Treatment Group | Hb (g/dL) | (or last value carried forward) |
| Compound A (n = 5) | 9.6 | 1.9 |
| Placebo (n = 3) | 9.8 | −0.35 |

*Difference between treatment and placebo group is statistically significant (Mann-Whitney rank sum test), p = 0.036.

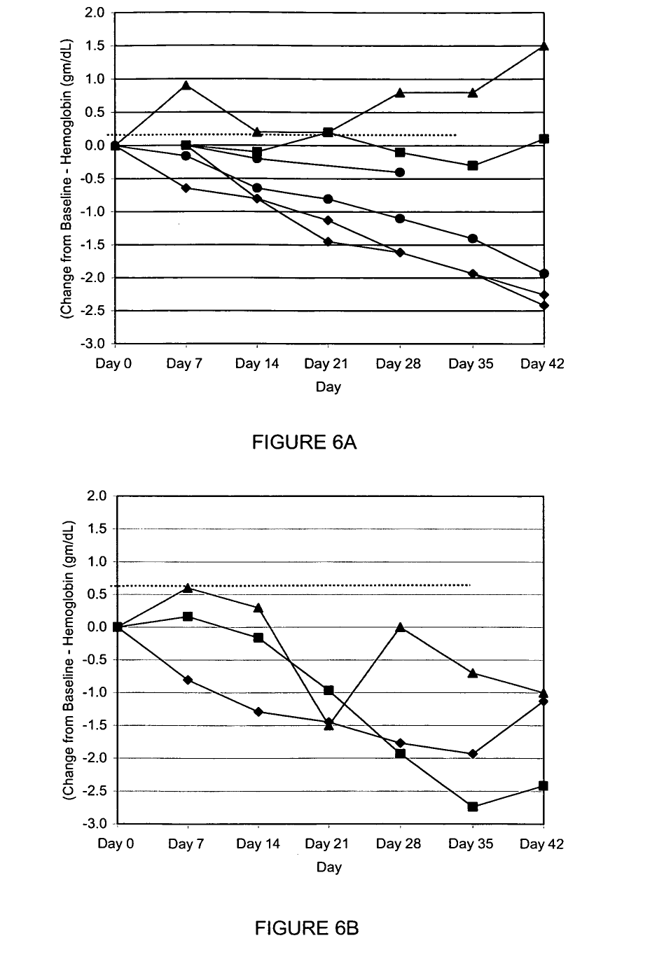

FIG. 6 shows the changes in Hb levels from baseline in rhEPO-treated subjects administered compound A (FIG. 6A) compared to the changes from baseline in placebo-treated subjects (FIG. 6B). As shown in Table 6 below, subjects administered compound A following cessation of rhEPO therapy showed a smaller change in mean baseline Hb levels (a 0.9 g/dL decrease from mean Hb baseline levels) than the change observed in subjects administered placebo (a 1.5 g/dL decrease from mean Hb baseline levels). This data indicated that methods of the present invention are useful for treating anemia in pre-dialysis subjects with chronic kidney disease.

TABLE 6

| | | Mean change from Baseline |
|---|---|---|
| | Mean Baseline | Hb (g/dL) Day 42 |
| Treatment Group | Hb (g/dL) | (or last value carried forward) |
| Compound (n = 6) | 11.7 | −0.9 |
| Placebo (n = 3) | 11.5 | −1.5 |

Taken together, these results also indicated that the methods of the present invention are useful for replacing rhEPO therapy or for use in conjunction with rhEPO treatment. Additionally, the desirable changes observed in hemoglobin (Hb) levels following administration of compound of the present invention were associated with circulating EPO levels well-below that observed following rhEPO treatment, indicating that therapeutic efficacy for treating anemia (e.g., increased Hb, increased hematocrit (Hct), etc.) are obtained with only minimal increases in circulating EPO levels. (Data not shown.)

### Example 7

### The Present Methods and Compounds Increase Circulating EPO Levels and Hemoglobin Levels in Mice

Mice were administered various doses (2 mg/kg, 6 mg/kg, 20 mg/kg, 60 mg/kg) of compounds of the present invention by oral gavage or by intravenous injection. Circulating levels of EPO were determined 15 6 hours after single-dose administration of compound. Hemoglobin levels were measured in mice on day 8 following administration of compound by oral gavage on day 1, day 3, and day 5. As shown in Table 7 below, both intravenous and oral gavage administration of compounds of the present invention increased circulating EPO levels in mice. As shown in Table 8 below, administration of compounds of the present invention three-times per week for one week increased hemoglobin levels in mice.

TABLE 7

| | EPO (mIU/ml) | EPO (mIU/ml) | EPO (mIU/ml) |
|---|---|---|---|
| Compound | Control | Intravenous | Oral Gavage |
| Cmpd A | 275 | 1309 | ND |
| Cmpd C | 0 | 1663 | ND |
| Cmpd D | 106 | 5473 | 1201 |
| Cmpd E | 106 | 2976 | 744 |
| Cmpd F | 107 | 3967 | ND |
| Cmpd G | 161 | 10969 | 2546 |
| Cmpd H | 107 | 1242 | 608 |

ND (not determined)

TABLE 8

| | | | | Hemoglobin | Hemoglobin |
|---|---|---|---|---|---|
| | Hemoglobin | Hemoglobin | Hemoglobin | (g/dL) | (g/dL) |
| | (g/dL) | (g/dL) | (g/dL) | 20 mg/kg | 60 mg/kg |
| Compound | Control | 2 mg/kg cmpd | 6 mg/kg cmpd | cmpd | cmpd |
| Cmpd A | 12.8 | ND | 12.86 | 12.92 | 13.47 |
| Cmpd C | 13 | DN | 13.68 | 14.29 | 14.94 |
| Cmpd D | 13.56 | 14.1 | 13.98 | 14.55q | ND |
| Cmpd E | 13.14 | 12.23 | 13.29 | 13.41 | 15.9 |
| Cmpd F | 12.93 | ND | 13.66 | 14.15 | 17.74 |
| Cmpd G | 12.83 | 14.39 | 13.13 | 14.85 | 17.7 |
| Cmpd H | 13.55 | 12.67 | 13.65 | 13.53 | 14.2 |

ND (not determined)

These results indicated that methods and compounds of the present invention are useful for increasing EPO and hemoglobin to therapeutically effective levels.

Various modifications of the invention, in addition to those shown and described herein, will become apparent to those skilled in the art from the foregoing description. Such modifications are intended to fall within the scope of the appended claims.

All references cited herein are hereby incorporated herein by reference in their entirety.

What is claimed is:

1. A method for treating anemia in a subject, the method comprising administering to the subject an effective amount of an agent that inhibits hypoxia inducible factor (HIF) hydroxylase activity, wherein the anemia treatment or the increased hemoglobin levels are associated with a lower risk of thrombosis or hypertension compared to that observed with recombinant human EPO therapy.

2. A method for increasing hemoglobin levels in a subject, the method comprising administering to the subject an effective amount of an agent that inhibits hypoxia inducible factor (HIF) hydroxylase activity, wherein the anemia treatment or the increased hemoglobin levels are associated with a lower risk of thrombosis or hypertension compared to that observed with recombinant human EPO therapy.

3. A method for increasing hematocrit in a subject, the method comprising administering to the subject an effective amount of an agent that inhibits hypoxia inducible factor (HIF) hydroxylase activity, wherein the anemia treatment or the increased hemoglobin levels are associated with a lower risk of thrombosis or hypertension compared to that observed with recombinant human EPO therapy.

4. The method of claim 1, wherein the agent is a compound that inhibits HIF prolyl hydroxylase activity.

5. The method of claim 1, wherein administration of the agent increases the circulating level of EPO in the subject to a level in the range of 10-100 mIU/ml.

6. The method of claim 1, wherein administration of the agent increases hemoglobin levels in the subject by a level in the range of 0.1-5.0 g/dL.

7. The method of claim 1, wherein administration of the agent increases hemoglobin levels in the subject to a level greater than a level selected from the group consisting of: 10 gm/dL, 11 gm/dL, 12 gm/dL, 13 gm/dL, and 14 gm/dL.

8. The method of claim 1, wherein administration of the agent increases hematocrit in the subject to a hematocrit selected from the group consisting of: 30%, 33%, 36%, 39%, and 42%.

9. The method of claim 1, wherein the agent is selected from a compound of Formula (I): 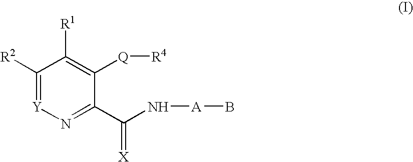
wherein
A is 1,2-arylidene, 1,3-arylidene, 1,4-arylidene; or (C₁-C₄)-alkylene, optionally substituted by one or two halogen, cyano, nitro, trifluoromethyl, (C₁-C₆)-alkyl, (C₁-C₆)-hydroxyalkyl, (C₁-C₆)-alkoxy, —O—[CH₂]ₓ—C_{f}H_{(2f+1-g)}Hal_{g}, (C₁-C₆)-fluoroalkoxy, (C₁-C₈)-fluoroalkenyloxy, (C₁-C₈)-fluoroalkynyloxy, —OCF₂Cl, —O—CF₂—CHFCl; (C₁-C₆)-alkylmercapto, (C₁-C₆)-alkylsulfinyl, (C₁-C₆)-alkylsulfonyl, (C₁-C₆)-alkylcarbonyl, (C₁-C₆)-alkoxycarbonyl, carbamoyl, N—(C₁-C₄)-alkylcarbamoyl, N,N-di-(C₁-C₄)-alkylcarbamoyl, (C₁-C₆)-alkylcarbonyloxy, (C₃-C₈)-cycloalkyl, phenyl, benzyl, phenoxy, benzyloxy, anilino, N-methylanilino, phenylmercapto, phenylsulfonyl, phenylsulfinyl, sulfamoyl, N—(C₁-C₄)-alkylsulfamoyl, N,N-di-(C₁-C₄)-alkylsulfamoyl; or by a substituted (C₆-C₁₂)-aryloxy, (C₇-C₁₁)-aralkyloxy, (C₆-C₁₂)-aryl, (C₇-C₁₁)-aralkyl radical, which carries in the aryl moiety one to five identical or different substituents selected from halogen, cyano, nitro, trifluoromethyl, (C₁-C₆)-alkyl, (C₁-C₆)-alkoxy, —O—[CH₂]ₓ—C_{f}H_{(2f+1-g)}Hal_{g}, —OCF₂Cl, —O—CF₂—CHFCl, (C₁-C₆)-alkylmercapto, (C₁-C₆)-alkylsulfinyl, (C₁-C₆)-alkylsulfonyl, (C₁-C₆)-alkylcarbonyl, (C₁-C₆)-alkoxycarbonyl, carbamoyl, N—(C₁-C₄)-alkylcarbamoyl, N,N-di-(C₁-C₄)-alkylcarbamoyl, (C₁-C₆)-alkylcarbonyloxy, (C₃-C₈)-cycloalkyl, sulfamoyl, N—(C₁-C₄)-alkylsulfamoyl, N,N-di-(C₁-C₄)-alkylsulfamoyl; or wherein A is —CR⁵R⁶ and R⁵ and R⁶ are each independently selected from hydrogen, (C₁-C₆)-alkyl, (C₃-C₇)-cycloalkyl, aryl, or a substituent of the α-carbon atom of an α-amino acid, wherein the amino acid is a natural L-amino acid or its D-isomer;
B is —CO₂H, —NH₂, —NHSO₂CF₃, tetrazolyl, imidazolyl, 3-hydroxyisoxazolyl, —CONHCOR′″, —CONHSOR′″, CONHSO₂R′″, where R′″ is aryl, heteroaryl, (C₃-C₇)-cycloalkyl, or (C₁-C₄)-alkyl, optionally monosubstituted by (C₆-C₁₂)-aryl, heteroaryl, OH, SH, (C₁-C₄)-alkyl, (C₁-C₄)-alkoxy, (C₁-C₄)-thioalkyl, (C₁-C₄)-sulfinyl, (C₁-C₄)-sulfonyl, CF₃, Cl, Br, F, I, N₂, —COOH, (C₂- C₅)-alkoxycarbonyl, NH₂, mono-(C₁-C₄-alkyl)-amino, di-(C₁-C₄-alkyl)-amino, or (C₁-C₄)-perfluoroalkyl; or wherein B is a CO₂-G carboxyl radical, where G is a radical of an alcohol G-OH in which G is selected from (C₁-C₂₀)-alkyl radical, (C₃-C₈) cycloalkyl radical, (C₂-C₂₀)-alkenyl radical, (C₃-C₈)-cycloalkenyl radical, retinyl radical, (C₂-C₂₀)-allcyyl radical, (C₄-C₂₀)-alkenynyl radical, where the alkenyl, cycloalkenyl, alkynyl, and alkenynyl radicals contain one or more multiple bonds; (C₆-C₁₆)-carbocyclic aryl radical, (C₇-C₁₆)-carbocyclic aralkyl radical, heteroaryl radical, or heteroaralkyl radical, wherein a heteroaryl radical or heteroaryl moiety of a heteroaralkyl radical contains 5 or 6 ring atoms; and wherein radicals defined for G are substituted by one or more hydroxyl, halogen, cyano, trifluoromethyl, nitro, carboxyl, (C₁-C₁₂)-alkyl, (C₃-C₈)-cycloalkyl, (C₅-C₈)-cycloalkenyl, (C₆-C₁₂)-aryl, (C₇-C₁₆)-aralkyl, (C₂-C₁₂)-alkenyl, (C₂-C₁₂)-alkynyl, (C₁-C₁₂)-alkoxy, (C₁-C₁₂)-alkoxy-(C₁-C₁₂)-alkyl, (C₁-C₁₂)-alkoxy-(C₁-C₁₂)-alkoxy, (C₆-C₁₂)-aryloxy, (C₇-C₁₆)-aralkyloxy, (C₁-C₈)-hydroxyalkyl, —O—[CH₂]ₓ—C_{f}H_{(2f+1-g)}—F_{g}, —OCF₂Cl, —OCF₂—CHFCl, (C₁-C₁₂)-alkylcarbonyl, (C₃-C₈)-cycloalkylcarbonyl, (C₆-C₁ 2)-arylcarbonyl, (C₇-C₁₆)-aralkylcarbonyl, cinnamoyl, (C₂-C₁₂)-alkenylcarbonyl, (C₂-C₁₂)-alkynylcarbonyl, (C₁-C₁₂)-alkoxycarbonyl, (C₁-C₁₂)-alkoxy-(C₁-C₁₂)-alkoxycarbonyl, (C₆-C₁₂)-aryloxycarbonyl, (C₇-C₁₆)-aralkyloxycarbonyl, (C₃-C₈)-cycloalkoxycarbonyl, (C₂-C₁₂)-alkenyloxycarbonyl, (C₂-C₁₂)-alkynyloxycarbonyl, acyloxy, (C₁-C₁₂)-alkoxycarbonyloxy, (C₁-C₁₂)-alkoxy-(C₁-C₁₂)-alkoxycarbonyloxy, (C₆-C₁₂)-aryloxycarbonyloxy, (C₇-C₁₆) aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N—(C₁-C₁₂)-alkylcarbamoyl, N.N-di(C₁-C₁₂)-alkylcarbamoyl, N—(C₃-C₈)-cycloalkyl-carbamoyl, N—(C₆-C₁₆)-arylcarbamoyl, N—(C₇-C₁₆)-aralkylcarbamoyl, N—(C₁-C₁₀)-alkyl-N—(C₆-C₁₆)-arylcarbamoyl, N—(C₁-C₁₀)-alkyl-N—(C₇-C₁₆)-aralkylcarbamoyl, N—((C₁-C₁₀)-alkoxy-(C₁-C₁₀)-alkyl)-carbamoyl, N—((C₆-C₁₂)-aryloxy-(C₁-C₁₀)alkyl)-carbamoyl, N—((C₇-C₁₆)-aralkyloxy-(C₁-C₁₀)-alkyl)-carbamoyl, N{C₁-C₁₀)-alkyl-N—((C₁-C₁₀)-alkoxy-(C₁-C₁₀)-alkyl)-carbamoyl,N—(C₁-C₁₀)-alkyl-N—((C₆-C₁₆)-aryloxy-(C₁-C₁₀)-alkyl)-carbamoyl, N—(C₁-C₁₀)-alkyl-N—((C₇-C₁₆)-aralkyloxy-(C₁-C₁₀)-alkyl)-carbamoyl, carbamoyloxy, N—(C₁-C₁₂)-alkylcarbamoyloxy, N.N-di-(C₁-C₁₂)-alkylcarbamoyloxy, N—(C₃-C₈)-cycloalkylcarbamoyloxy, N—(C₆-C₁₂)-arylcarbamoyloxy, N—(C₇-C₁₆)-aralkylcarbamoyloxy, N—(C₁-C₁₀)-alkyl-N—(C₆-C₁₂)-arylcarbamoyloxy, N(C₁-C₁₀)-alkyl-N—(C₇-C₁₆)-aralkylcarbamoyloxy, N—(C₁-C₁₀)-alkyl)-carbamoyloxy, N—((C₆-C₁₂)-aryloxy-(C₁-C₁₀)-alkyl)-carbamoyloxy, N—((C₇-C₁₆)-aralkyloxy-(C₁-C₁₀)-alkyl)-carbamoyloxy, N—(C₁-C₁₀)-alkyl-N—((C₁-C₁₀)-alkoxy-(C₁-C₁₀)-alkyl)-carbamoyloxy,N—(C₁-C₂₁)-alkyl-N—((C₆-C₁₂)-aryloxy-(C₁-C₁₀)-alkyl)-carbamoyloxy, N—(C₁-C₁₀)-alkyl-N—((C₇-C₁₆)-aralkyloxy-(C₁-C₁₀)-alkyl)-carbamoyloxy, amino, (C₁-C₁₂)-alkylamino, di-(C₁-C₁₂)-alkylamino, (C₃-C₈)-cycloalkylamino, (C₂-C₁₂)-alkenylamino, (C₂-C₁₂)-alkynylamino, N—(C₆-C₁₂)-arylamino, N—(C-C₁₁)-aralkylamino, N-alkyl-aralkylamino, N-alkyl-arylamino, (C₁-C₁₂)-alkoxyamino, (C₁-C₁₂)-alkoxy-N—(C₁-C₁₀)-alkylamino, (C₁-C₁₂)-alkylcarbonylamino, (C₃-C₈)-cycloalkylcarbonylamino, (C₆-C₁₂) arylcarbonylamino, (C₇-C₁₆)-aralkylcarbonylamino, (C₁-C₁₂)-alkylcarbonyl-N—(C₁-C₁₀)-alkylamino, (C₃-C₈)-cycloalkylcarbonyl-N—(C₁-C₁₀)-alkylamino, (C₆-C₁₂)-arylcarbonyl-N—(C₁-C₁₀)alkylamino, (C₇-C₁₁)-aralkylcarbonyl-N—(C₁-C₁₀)-alkylamino, (C₁-C₁₂)-alkylcarbonylamino-(C₁-C₈)-alkyl, (C₃-C₈)-cycloalkylcarbonylamino-(C₁-C₈)alkyl, (C₆-C₁₂)-arylcarbonylamino-(C₁-C₈)-alkyl, (C₇-C₁₂)-aralkylcarbonylamino(C₁-C₈)-alkyl, amino-(C₁-C₁₀)-alkyl, N—(C₁-C₁₀) alkylamino-(C₁-C₁₀)-alkyl, N.N-di (C₁-C₁₀)-alkylamino-(C₁-C₁₀)-alkyl, (C₃-C₈)cycloalkylamino-(C₁-C₁₀)-alkyl, (C₁-C₁₂)-alkylmercapto, (C₁-C₁₂)-alkylsulfinyl, (C₁-C₁₂)-alkylsulfonyl, (C₆-C₁₆)-arylmercapto, (C₆-C₁₆)-arylsulfinyl, (C₆-C₁₂)-arylsulfonyl, (C₇-C₁₆)-aralkylmercapto, (C₇-C₁₆)-aralkylsulfinyl, (C₇-C₁₆)-aralkylsulfonyl, sulfamoyl, N—(C₁-C₁₀)-alkylsulfamoyl, N.N-di(C₁-C₁₀)-alkylsulfamoyl, (C₃-C₈)-cycloalkylsulfamoyl, N—(C₆-C₁₂)-alkylsulfamoyl, N—(C₇-C₁₆)-aralkylsulfamoyl, N—(C₁-C₁₀)-alkyl-N—(C₆-C₁₂)-arylsulfamoyl, N—(C₁-C₁₀)-alkyl-N—(C₇-C₁₆)-aralkylsulfamoyl, (C₁-C₁₀)-alkylsulfonamido, N—((C₁-C₁₀)-alkyl)-(C₁-C₁₀)-alkylsulfonamido, (C₇-C₁₆)-aralkylsulfonamido, or N—((C₁-C₁₀)-alkyl-(C₇-C₁₆)-aralkylsulfonamido; wherein radicals which are aryl or contain an aryl moiety, may be substituted on the aryl by one to five identical or different hydroxyl, halogen, cyano, trifluoromethyl, nitro, carboxyl, (C₁-C₁₂)-alkyl, (C₃-C₈)-cycloalkyl, (C₆-C₁₂)-aryl, (C₇-C₁₆)-aralkyl, (C₁-C₁₂)-alkoxy, (C₁-C₁₂)-alkoxy-(C₁-C₁₂)alkyl, (C₁-C₁₂)-alkoxy-(C₁-C₁₂)alkoxy, (C₆-C₁₂)-aryloxy, (C₇-C₁₆)-aralkyloxy, (C₁-C₈)-hydroxyalkyl, (C₁-C₁₂)-alkylcarbonyl, (C₃-C₈)-cycloalkyl-carbonyl, (C₆-C₁₂)-arylcarbonyl, (C₇-C₁₆) aralkylcarbonyl, (C₁-C₁₂)-alkoxycarbonyl, (C₁-C₁₂)-alkoxy-(C₁-C₁₂)-alkoxycarbonyl, (C₆-C₁₂)-aryloxycarbonyl, (C₇-C₁₆)-aralkyloxycarbonyl, (C₃-C₈)-cycloalkoxycarbonyl, (C₂-C₁₂)-alkenyloxycarbonyl, (C₂-C₁₂)-alkynyloxycarbonyl, (C₁-C₁₂)-alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₁-C₁₂)-alkoxy-(C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N—(C₁-C₁₂)-alkylcarbamoyl, N.N-di-(C₁-C₁₂)-alkylcarbamoyl, N—(C₃-C₈)-cycloalkylcarbamoyl, N—(C₆-C₁₂)-arylcarbamoyl, N—(C₇-C₁₆)-aralkylcarbamoyl, N—(C₁-C₁₀)-alkyl-N—(C₆-C₁₂)-arylcarbamoyl, N—(C₁-C₁₀)-alkyl-N—(C₇-C₁₆)-aralkylcarbamoyl, N—((C₁-C₁₀)-alkoxy-(C₁-C₁₀)-alkyl)-carbamoyl, N—((C₆-C₁₂)-aryloxy-(C₁-C₁₀)-alkyl)-carbamoyl, N—((C₇-C₁₆)-aralkyloxy-(C₁-C₁₀)-alkyl)-carbamoyl,N—(C₁-C₁₀)-alkyl-N—((C₁-C₁₀)-alkoxy-(C₁-C₁₀)-alkyl)-carbamoyl, N—(C₁-C₁₀)-alkyl-N—((C₆-C₁₂)-aryloxy-(C₁-C₁₀)-alkyl)-carbamoyl, N—(C₁-C₁₀)-alkyl-N—((C₇-C₁₆)-aralkyloxy-(C₁-C₁₀)-alkyl)-carbamoyl, carbamoyloxy, N—(C₁-C₁₂)-alkylcarbamoyloxy, N.N-di-(C₁-C₁₂)-alkylcarbamoyloxy, N—(C₃-C₈)-cycloalkylcarbamoyloxy, N—(C₆-C₁₂)-arylcarbamoyloxy, N—(C₇-C₁₆)-aralkylcarbamoyloxy, N—(C₁-C₁₀)-alkyl-N—(C₆-C₁₂)-arylcarbamoyloxy, N(C₁-C₁₀)-alkyl-N—(C₇-C₁₆)-aralkylcarbamoyloxy, N—((C₁-C₁₀)-alkyl)-carbamoyloxy, N—((C₆-C₁₂)-aryloxy-(C₁-C₁₀)-alkyl)-carbamoyloxy, N—((C₇-C₁₆)-aralkyloxy-(C₁-C₁₀)-alkyl)-carbamoyloxy, N—(C₁-C₁₀)-alkyl-N—((C₁-C₁₀)-alkoxy-(C₁-C₁₀)-alkyl)-carbamoyloxy, N—(C₁-C₁₀)-alkyl-N—((C₆-C₁₂)-aryloxy-(C₁-C₁₀)-alkyl)-carbamoyloxy, N—(C₁-C₁₀)-alkyl-N—((C₇-C₁₆)-aralkyloxy-(C₁-C₁₀)-alkyl)-carbamoyloxy, amino, (C₁-C₁₂)-alkylamino, di-(C₁-C₁₂)-alkylamino, (C₃-C₈)-cycloalkylamino, (C₃-C₁₂)-alkenylamino, (C₃-C₁₂)-alkynylamino, N—(C₆-C₁₂)-arylamino, N—(C₇-C₁₁)-aralkylamino, N-alkylaralkylamino, N-alkyl-arylamino, (C₁-C₁₂)-alkoxyamino, (C₁-C₁₂)-alkoxy-N—(C₁-C₁₀)-alkylamino, (C₁-C₁₂)-alkylcarbonylamino, (C₃-C₈)-cycloalkylcarbonylamino, (C₆-C₁₂)-arylcarbonylamino, (C₇-C₁₆)-alkylcarbonylamino, (C₁-C₁₂)-alkylcarbonyl-N—(C₁-C₁₀)-alkylamino, (C₃-C₈)-cycloalkylcarbonyl-N—(C₁-C₁₀)-alkylamino, (C₆-C₁₂)-arylcarbonyl-N—(C₁-C₁₀)-alkylamino, (C₇-C₁₁)-aralkylcarbonyl-N—(C₁-C₁₀)-alkylamino, (C₁-C₁₂)-alkylcarbonylamino-(C₁-C₈)-alkyl, (C₃-C₈)-cycloalkylcarbonylamino-(C₁-C₈)-alkyl, (C₆-C₁₂)-arylcarbonylamino-(C₁-C₈)-alkyl, (C₇-C₁₆)-aralkylcarbonylamino-(C₁-C₈)-alkyl, amino-(C₁-C₁₀)-alkyl, N—(C₁-C₁₀)-alkylamino-(C₁-C₁₀)alkyl, N.N-di-(C₁-C₁₀)-alkylamino-(C₁-C₁₀)-alkyl, (C₃-C₈)-cycloalkylamino-(C₁-C₁₀)-alkyl, (C₁-C₁₂)-alkylmercapto, (C₁-C₁₂)-alkylsulfinyl, (C₁-C₁₂)-alkylsulfonyl, (C₆-C₁₂)-arylmercapto, (C₆-C₁₂)-arylsulfinyl, (C₆-C₁₂)-arylsulfonyl, (C₇-C₁₆)-aralkylmercapto, (C₇-C₁₆)-aralkylsulfinyl, or (C₇-C₁₆)-aralkylsulfonyl;
X is O or S;
Q is O, S, NR′, or a bond;
where, if Q is a bond, R⁴ is halogen, nitrile, or trifluoromethyl;
or where, if Q is O, S, or NR′, R⁴ is hydrogen, (C₁-C₁₀)-alkyl radical, (C₂-C₁₀)-alkenyl radical, (C₂-C₁₀)-alkynyl radical, wherein alkenyl or alkynyl radical contains one or two C—C multiple bonds; unsubstituted fluoroalkyl radical of the formula —[CH₂]ₓ—C_{f}H_{(2f+1-g)}—-F_{g}, (C₁-C₈)-alkoxy-(C₁-C₆)-alkyl radical, (C₁-C₆)-alkoxy-(C₁-C₄)-alkoxy-(C₁-C₄)-alkyl radical, aryl radical, heteroaryl radical, (C₇-C₁₁)-aralkyl radical, or a radical of the formula Z
—[CH₂]ᵥ—[O]_{w}—[CH₂]ₜ-E (Z)
where
E is a heteroaryl radical, a (C₃-C₈)-cycloalkyl radical, or a phenyl radical of the formula F 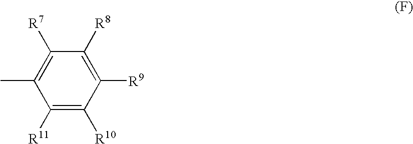
v is 0-6,
w is 0 or 1,
t is 0-3, and
R⁷, R⁸, R⁹, R¹⁰, and R¹¹ are identical or different and are hydrogen, halogen, cyano, nitro, trifluoromethyl, (C₁-C₆)-alkyl, (C₃-C₈)-cycloalkyl, (C₁-C₆)-alkoxy, —O—[CH₂]ₓ—C_{f}H_{(2f+1-g)}—F_{g}, -0CF₂—Cl, —O—CF₂—CHFCl, (C1-C6)-alkylmercapto, (C1-C6)-hydroxyalkyl, (C1-C6)-alkoxy-(C1-C₆)-alkoxy, (C₁-C₆)-alkoxy-(C₁-C₆)-alkyl, (C₁-C₆)-alkylsulfinyl, (C₁-C₆)-alkylsulfonyl, (C₁-C₆)-alkylcarbonyl, (C₁-C₈)-alkoxycarbonyl, carbamoyl, N—(C₁-C₈)-alkylcarbamoyl, N,N-di-(C₁-C₈)-alkylcarbamoyl, or (C₇-C₁₁)-aralkylcarbamoyl, optionally substituted by fluorine, chlorine, bromine, trifluoromethyl, (C₁-C₆)-alkoxy, N—(C₃-C₈)-cycloalkylcarbamoyl, N—(C₃-C₈)-cycloalkyl-(C₁-C₄)-alkylcarbamoyl, (C₁-C₆)-alkylcarbonyloxy, phenyl, benzyl, phenoxy, benzyloxy, NR^{Y}R^{Z} wherein R^{y} and R^{z} are independently selected from hydrogen, (C₁-C₁₂)-alkyl, (C₁-C₈)-alkoxy-(C₁-C₈)-alkyl, (C₇-C₁₂)-aralkoxy-(C₁-C₈)-alkyl, (C₆-C₁₂)-aryloxy-(C₁-C₈)-alkyl, (C₃-C₁₀)-cycloalkyl, (C₃-C₁₂)-alkenyl, (C₃-C₁₂)-alkynyl, (C₆-C₁₂)-aryl, (C₇-C₁₁)-aralkyl, (C₁-C₁₂)-alkoxy, (C₇-C₁₂)aralkoxy, (C₁-C₁₂)-alkylcarbonyl, (C₃-C₈)-cycloalkylcarbonyl, (C₆-C₁₂) arylcarbonyl, (C₇-₁₆)-aralkylcarbonyl; or further wherein R^{y} and R^{z} together are —[CH₂]ₕ, in which a CH₂ group can be replaced by O, S, N—(C₁-C₄)-alkylcarbonylimino, or N—(C₁-C₄)-alkoxycarbonylimino; phenylmercapto, phenylsulfonyl, phenylsulfinyl, sulfamoyl, N—(C₁-C₈)-alkylsulfamoyl, or N,N-di-(C₁-C₈)-alkylsulfamoyl; or alternatively R⁷ and R⁸, R⁸ and R⁹, R⁹ and R¹⁰, or R¹⁰ and R¹¹, together are a chain selected from —[CH₂]ₙ— or —CH═CH—CH═CH-, where a CH₂ group of the chain is optionally replaced by O, S, SO, SO₂, or NR^{Y}; and n is 3,4, or 5; and if E is a heteroaryl radical, said radical can carry 1-3 substituents selected from those defined for R⁷—R¹¹, or if E is a cycloalkyl radical, the radical can carry one substituent selected from those defined for R⁷—R¹¹;
or where, if Q is NR′, R⁴ is alternatively R″, where R′ and R″ are identical or different and are hydrogen, (C₆-C₁₂)-aryl, (C₇-C₁₁)-aralkyl, (C₁-C₈)-alkyl, (C₁-C₈)-alkoxy-(C₁-C₈)-alkyl, (C₇-C₁₂)-aralkoxy-(C₁-C₈)-alkyl, (C₆-C₁₂)-aryloxy-(C₁-C₈)-alkyl, (C₁-C₁₀)-alkylcarbonyl, optionally substituted (C₇-C₁₆)-aralkylcarbonyl, or optionally substituted C₆-C₁₂)-arylcarbonyl; or R′ and R″ together are —[CH₂]ₕ, in which a CH₂ group can be replaced by O, S, N-acylimino, or N—(C₁-C₁₀)-alkoxycarbonylimino, and h is 3 to 7.
Y is N or CR³;
R¹, R² and R³ are identical or different and are hydrogen, hydroxyl, halogen, cyano, trifluoromethyl, nitro, carboxyl, (C₁-C₂₀)-alkyl, (C₃-C₈)-cycloalkyl, (C₃-C₈)cycloalkyl-(C₁-C₁₂)-alkyl, (C₃-C₈)-cycloalkoxy, (C₃-C₈)-cycloalkyl-(C₁-C₁₂)-alkoxy, (C₃-C₈)-cycloalkyloxy-(C₁-C₁₂)-alkyl, (C₃-C₈)-cycloalkyloxy-(C₁-C₁₂)-alkoxy, (C₃-C₈)-cycloalkyl-(C₁-C₈)-alkyl-(C₁-C₆)-alkoxy, (C₃-C₈)-cycloalkyl-(C₁-C₈)-alkoxy-(C₁-C₆)-alkyl, (C₃-C₈)-cycloalkyloxy-(C₁-C₈)-alkoxy-(C₁-C₁₆)-alkyl, (C₃-C₈)-cycloalkoxy-(C₁-C₈)-alkoxy-(C₁-C₈)-alkoxy, (C₆-C₁₂)-aryl, (C₇-C₁₆)-aralkyl, (C₇-C₁₆)-aralkenyl, (C₇-C₁₆)-aralkynyl, (C₂-C₂₀)-alkenyl, (C₂-C₂₀)-alkynyl, (C₁-C₂₀)-alkoxy, (C₂-C₂₀)-alkenyloxy, (C₂-C₂₀)-alkynyloxy, retinyloxy, (C₁-C₂₀)-alkoxy-(C₁-C₁₂)-alkyl, (C₁-C₁₂)-alkoxy-(C₁-C₁₂)-alkoxy, (C₁-C₁₂)-alkoxy-(C₁-C₈)-alkoxy-(C₁-C₈)-alkyl, (C₆-C₁₂)-aryloxy, (C₇-C₁₆)-aralkyloxy, (C₆-C₁₂)-aryloxy-(C₁-C₆)-alkoxy, (C₇-C₁₆)-aralkoxy-(C₁-C₆)-alkoxy, (C₁-C₁₆)-hydroxyalkyl, (C₆-C₁₆)-aryloxy-(C₁-C₈)-alkyl, (C₇-C₁₆)-aralkoxy-(C₁-C₈)-alkyl, (C₆-C₁₂)-aryloxy-(C₁-C₈)-alkoxy-(C₁-C₆)-alkyl, (C₇-C₁₂)-aralkyloxy-(C₁-C₈)-alkoxy-(C₁-C₆)-alkyl, (C₂-C₂₀)-alkenyloxy-(C₁-C₆)-alkyl, (C₂-C₂₀)-alkynyloxy-(C₁-C₆)-alkyl, retinyloxy-(C₁-C₆)-alkyl, —O—[CH₂]ₓC_{f}H_{(2f+1-g)}F_{g}, —OCF₂Cl, —OCF₂—CHFCl, (C₁-C₂₀)-alkylcarbonyl, (C₃-C₈)-cycloalkylcarbonyl, (C₆-C₁₂)-arylcarbonyl, (C₇-C₁₆)-aralkylcarbonyl, cinnamoyl, (C₂-C₂₀)-alkenylcarbonyl, (C₂-C₂₀)-alkynylcarbonyl, (C₁-C₂₀)-alkoxycarbonyl, (C₁-C₁₂)-alkoxy-(C₁-C₁₂)-alkoxycarbonyl, (C₆-C₁₂)-aryloxycarbonyl, (C₇-C₁₆)-aralkyloxycarbonyl, (C₃-C₈)-cycloalkoxycarbonyl, (C₂-C₂₀)-alkenyloxycarbonyl, retinyloxycarbonyl, (C₂-C₂₀)-alkynyloxycarbonyl, (C₆-C₁₂)-aryloxy-(C₁-C₆)-alkoxycarbonyl, (C₇-C₁₆)-aralkoxy-(C₁-C₆)-alkoxycarbonyl, (C₃-C₈)-cycloalkyl-(C₁-C₆)-alkoxycarbonyl, (C₃-C₈)-cycloalkoxy-(C₁-C₆)-alkoxycarbonyl, (C₁-C₁₂)-alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₁-C₁₂)-alkoxy-(C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N—(C₁-C₁₂)-alkylcarbamoyl, N,N-di-(C₁-C₁₂)-alkylcarbamoyl, N—(C₃-C₈)-cycloalkylcarbamoyl, N,N-dicyclo-(C₃-C₈)-alkylcarbamoyl, N—(C₁-C₁₀)-alkyl-N—(C₃-C₈)-cycloalkylcarbamoyl, N—((C₃-C₈)-cycloalkyl-(C₁-C₆)-alkyl)-carbamoyl, N—(C₁-C₆)-alkyl-N—((C₃-C₈)-cycloalkyl-(C₁-C₆)-alkyl)-carbamoyl, N-(+)-dehydroabietylcarbamoyl, N—(C₁-C₆)-alkyl-N-(+)-dehydroabietylcarbamoyl, N—(C₆-C₁₂)-arylcarbamoyl, N—(C₇-C₁₆)-aralkylcarbamoyl, N—(C₁-C₁₀)-alkyl-N—(C₆-C₁₆)-arylcarbamoyl, N—(C₁-C₁₀)-alkyl-N—(C₇-C₁₆)-aralkylcarbamoyl,N—((C₁-C₁₈)-alkoxy-(C₁-C₁₀)-alkyl)-carbamoyl, N—((C₆-C₁₆)-aryloxy-(C₁-C₁₀)-alkyl)-carbamoyl, N—((C₇-C₁₆)-aralkyloxy-(C₁-C₁₀)-alkyl)-carbamoyl, N—(C₁-C₁₀)-alkyl-N—((C₁-C₁₀)-alkoxy-(C₁₀)-alkyl)-carbamoyl, N—(C₁-C₁₀)-alkyl-N—((C₆-C₁₂)-aryloxy-(C₁-C₁₀)-alkyl)-carbamoyl, N—(C₁-C₁₀)-alkyl-N—((C₇-C₁₆)-aralkyloxy-(C₁-C₁₀)-alkyl)-carbamoyl; CON(CH₂)ₕ, in which a CH₂ group can be replaced by O, S, N—(C₁-C₈)-alkylimino, N—(C₃-C₈)-cycloalkylimino, N—(C₃-C₈)-cycloalkyl-(C₁-C₄)-alkylimino, N—(C₆-C₁₂)-arylimino, N—(C₇-C₁₆)-aralkylimino, N—(C₁-C₄)-alkoxy-(C₁-C₆)-alkylimino, and h is from 3 to 7; a carbamoyl radical of the formula R 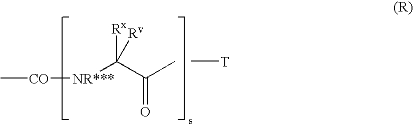
in which
R^{x} and R^{v} are each independently selected from hydrogen, (C₁-C₆)-alkyl, (C₃-C₇)-cycloalkyl, aryl, or the substituent of an α-carbon of an α-amino acid, to which the L- and D-amino acids belong,
s is 1-5,
T is OH, or NR*R**, and R*, R** and R*** are identical or different and are selected from hydrogen, (C₆-C₁₂)-aryl, (C₇-C₁₁)-aralkyl, (C₁-C₈)-alkyl, (C₃-C₈)-cycloalkyl, (+)-dehydroabietyl, (C₁-C₈)-alkoxy-(C₁-C₈)-alkyl, (C₇-C₁₂)-aralkoxy-(C₁-C₈)-alkyl, (C₆-C₁₂)-aryloxy-(C₁-C₈)-alkyl, (C₁-C₁₀)-alkanoyl, optionally substituted (C₇-C₁₆)-aralkanoyl, optionally substituted (C₆-C₁₂)-aroyl; or R* and R** together are -[CH₂]h, in which a CH₂ group can be replaced by O, S, SO, SO₂, N-acylamino, N—(C₁-C₁₀)-alkoxycarbonylimino, N—(C₁-C₈)-alkylimino, N—(C₃-C₈)-cycloalkylimino, N—(C₃-C₈)-cycloalkyl-(C₁-C₄)-alkylimino, N—(C₆-C₁₂)-arylimino, N—(C₇-C₁₆)-arakylimino, N—(C₁-C₄)-alkoxy-(C₁-C₆)-alkylimino, and h is from 3 to 7;
carbamoyloxy, N—(C₁-C₁₂)-alkylcarbamoyloxy, N,N-di-(C₁-C₁₂)-alkylcarbamoyloxy, N—(C₃-C₈)-cycloalkylcarbamoyloxy, N—(C₆-C₁₂)-arylcarbamoyloxy, N—(C₇-C₁₆)-aralkylcarbamoyloxy, N—(C₁-C₁₀)-alkyl-N—(C₆-C₁₂)-arylcarbamoyloxy,N—(C₁-C₁₀)-alkyl-N—(C₇-C₁₆)-aralkylcarbamoyloxy, N- ((C₁-C₁₀)-alkyl)-carbamoyloxy, N—((C₆-C₁₂)-aryloxy-(C₁-C₁₀)-alkyl)-carbamoyloxy, N—((C₇-C₁₆)-aralkyloxy-(C₁-C₁₀)-alkyl)-Carbamoyloxy, N—(C₁-C₁₀)-alkyl-N—((C₁-C₁₀)-alkoxy-(C₁-C₁₀)-alkyl)-carbamoyloxy, N—(C₁-C₁₀)-alkyl-N—((C₆-C₁₂)-aryloxy-(C₁-C₁₀)-alkyl)-carbamoyloxy, N—(C₁-C₁₀)-alkyl-N—((C₇-C₁₆)-aralkyloxy-(C₁-C₁₀)-alkyl)-carbamoyloxyamino, (C₁-C₁₂)-alkylamino, di-(C₁-C₁₂)-alkylamino, (C₃-C₈)-cycloalkylamino, (C₃-C₁₂)-alkenylamino, (C₃-C₁₂)-alkynylamino, N—(C₆-C₁₂)-arylamino, N—(C₇-C₁₁)-aralkylamino, N-alkyl-aralkylamino, N-alkyl-arylamino, (C₁- C₁₂)-alkoxyamino, (C₁-C₁₂)-alkoxy-N—(C₁-C₁₀)-alkylamino, (C₁-C₁₂)-alkanoylamino, (C₃-C₈)-cycloalkanoylamino, (C₆-C₁₂)-aroylamino, (C₇-C₁₆)-aralkanoylamino, (C₁-C₁₂)-alkanoyl-N—(C₁-C₁₀)-alkylamino, (C₃-C₈)-cycloalkanoyl-N—(C₁-C₁₀)-alkylamino, (C₆-C₁₂)-aroyl-N—(C₁-C₁₀)-alkylamino, (C₇-C₁₁)-aralkanoyl-N—(C₁-C₁₀)-alkylamino, (C₁-C₁₂)-alkanoylamino-(C₁-C₈)-alkyl, (C₃-C₈)-cycloalkanoylamino-(C₁-C₈)-alkyl, (C₆-C₁₂)-aroylamino-(C₁-C₈)-alkyl, (C₇-C₁₆)-aralkanoylamino-(C₁-C₈)-alkyl, amino-(C₁-C₁₀)-alkyl, N—(C₁-C₁₀)-alkylamino-(C₁-C₁₀)-alkyl, N,N-di(C₁-C₁₀)-alkylamino-(C₁-C₁₀)-alkyl, (C₃-C₈)-cycloalkylamino(C₁-C₁₀)-alkyl, (C₁-C₂₀)-alkylmercapto, (C₁-C₂₀)-alkylsulfinyl, (C₁-C₂₀)-alkylsulfonyl, (C₆-C₁₂)-arylmercapto, (C₆-C₁₂)-arylsulfinyl, (C₆-C₁₂)-arylsulfonyl, (C₇-C₁₆)-aralkylmercapto, (C₇-C₁₆)-aralkylsulfinyl, (C₇-C₁₆)-aralkylsulfonyl, (C₁-C₁₂)-alkylmercapto-(C₁-C₆)-alkyl, (C₁-C₁₂)-alkylsulfinyl-(C₁-C₆)-alkyl, (C₁-C₁₂)-alkylsulfonyl-(C₁-C₆)-alkyl, (C₆-C₁₂)-arylmercapto-(C₁-C₆)-alkyl, (C₆-C₁₂)-arylsulfinyl-C₁-C₆)-alkyl, (C₆-C₁₂)-arylsulfonyl-(C₁-C₆)-alkyl, (C₇-C₁₆)-aralkylmercapto-(C₁-C₆)-alkyl, (C₇-C₁₆)-aralkylsulfinyl-(C₁-C₆)-alkyl, (C₇-C₁₆)-aralkylsulfonyl-(C₁-C₆)-alkyl, sulfamoyl, N—(C₁-C₁₀)-alkylsulfamoyl, N,N-di-(C₁-C₁₀)-alkylsulfamoyl, (C₃-C₈)-cycloalkylsulfamoyl, N—(C₆-C₁₂)-arylsulfamoyl, N—(C₇-C₁₆)-aralkylsulfamoyl, N—(C₁-C₁₀)-alkyl-N—(C₆-C₁₂)-arylsulfamoyl, N—(C₁-C₁₀)-alkyl-N—(C₇-C₁₆)-aralkylsulfamoyl, (C₁-C₁₀)-alkylsulfonamido, N—((C₁-C₁₀)-alkyl)-(C₁-C₁₀)-alkylsulfonamido, (C₇-C₁₆)-aralkylsulfonamido, and N—((C₁-C₁₀)-alkyl-(C₇-C₁₆)-aralkylsulfonamido; where an aryl radical may be substituted by 1 to 5 substituents selected from hydroxyl, halogen, cyano, trifluoromethyl, nitro, carboxyl, (C₂-C₁₆)-alkyl, (C₃-C₈)-cycloalkyl, (C₃-C₈)-cycloalkyl-(C₁-C₁₂)-alkyl, (C₃-C₈)-cycloalkoxy, (C₃-C₈)-cycloalkyl-(C₁-C₁₂)-alkoxy, (C₃-C₈)-cycloalkyloxy-(C₁-C₁₂)-alkyl, (C₃-C₈)-cycloalkyloxy-(C₁-C₁₂)-alkoxy, (C₃-C₈)-cycloalkyl-(C₁-C₈)-alkyl-(C₁-C₆)-alkoxy, (C₃-C₈)-cycloalkyl(C₁-C₈)-alkoxy-(C₁-C₆)-alkyl, (C₃-C₈)-cycloalkyloxy-(C₁-C₈)-alkoxy-(C₁-C₆)-alkyl, (C₃-C₈)-cycloalkoxy-(C₁-C₈)-alkoxy-(C₁-C₈)-alkoxy, (C₆-C₁₂)-aryl, (C₇-C₁₆)-aralkyl, (C₂-C₁₆)-alkenyl, (C₂-C₁₂)-alkynyl, (C₁-C₁₆)-alkoxy, (C₁-C₁₆)-alkenyloxy, (C₁-C₁₂)-alkoxy-(C₁-C₁₂)-alkyl, (C₁-C₁₂)-alkoxy-(C₁-C₁₂)-alkoxy, (C₁-C₁₂)-alkoxy(C₁-C₈)-alkoxy-(C₁-C₈)-alkyl, (C₆-C₁₂)-aryloxy, (C₇-C₁₆)-aralkyloxy, (C₆-C₁₂)-aryloxy-(C₁-C₆)-alkoxy, (C₇-C₁₆)-aralkoxy-(C₁-C₆)-alkoxy, (C₁-C₈)-hydroxyalkyl, (C₆-C₁₆)-aryloxy-(C₁-C₈)-alkyl, (C₇-C₁₆)-aralkoxy-(C₁-C₈)-alkyl, (C₆-C₁₂)-aryloxy-(C₁-C₈)-alkoxy-(C₁-C₆)-alkyl, (C₇-C₁₂)-aralkyloxy-(C₁-C₈)-alkoxy-(C₁-C₆)-alkyl, —O—[CH₂]ₓC_{f}H_{(2f+1-g)}F_{g}, —OCF₂Cl, —OCF₂—CHFCl,(C₁-C₁₂)-alkylcarbonyl, (C₃-C₈)-cycloalkylcarbonyl, (C₆-C₁₂)-arylcarbonyl, (C₇-C₁₆)-aralkylcarbonyl, (C₁-C₁₂)-alkoxycarbonyl, (C₁-C₁₂)-alkoxy-(C₁-C₁₂)-alkoxycarbonyl, (C₆-C₁₂)-aryloxycarbonyl, (C₇-C₁₆)-aralkyloxycarbonyl, (C₃-C₈)-cycloalkoxycarbonyl, (C₂-C₁₂)-alkenyloxycarbonyl, (C₂-C₁₂)-alkynyloxycarbonyl, (C₆-C₁₂)-aryloxy-(C₁-C₆)-alkoxycarbonyl, (C₇-C₁₆)-aralkoxy-(C₁-C₆)-alkoxycarbonyl, (C₃-C₈)-cycloalkyl-(C₁-C₆)-alkoxycarbonyl, (C₃-C₈)-cycloalkoxy-(C₁-C₆)-alkoxycarbonyl, (C₁-C₁₂)-alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₁-C₁₂)-alkoxy-(C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N—(C₁-C₁₂)-alkylcarbamoyl, N,N-di(C₁-C₁₂)-alkylcarbamoyl, N—(C₃-C₈)-cycloalkylcarbamoyl, N,N-dicyclo-(C₃-C₈)-alkylcarbamoyl, N—(C₁-C₁₀)-alkyl-N—(C₃-C₈)-cycloalkylcarbamoyl, N—((C₃-C₈)-cycloalkyl-(C₁-C₆)-alkyl)carbamoyl, N—(C₁-C₆)-alkyl-N—((C₃-C₈)-cycloalkyl-(C₁-C₆)-alkyl)carbamoyl, N-(+)-dehydroabietylcarbamoyl, N—(C₁-C₆)-alkyl-N-(+)-dehydroabietylcarbamoyl, N—(C₆-C₁₂)-arylcarbamoyl, N—(C₇-C₁₆)-aralkylcarbamoyl, N—(C₁-C₁₀)-alkyl-N—(C₆-C₁₆)-arylcarbamoyl, N—(C₁-C₁₀)-alkyl-N—(C₇-C₁₆)-aralkylcarbamoyl, N—((C₁-C₁₆)-alkoxy-(C₁-C₁₀)-alkyl)carbamoyl, N—((C₆-C₁₆)-aryloxy-(C₁-C₁₀)-alkyl)carbamoyl, N—((C₇-C₁₆)-aralkyloxy-(C₁-C₁₀)-alkyl)carbamoyl, N—(C₁-C₁₀)-alkyl-N—((C₁-C₁₀)-alkoxy-(C₁-C₁₀)-alkyl) carbamoyl, N—(C₁-C₁₀)-alkyl-N—((C₆-C₁₂)-aryloxy-(C₁-C₁₀)-alkyl)carbamoyl, N—(C₁-C₁₀)-alkyl-N—((C₇-C₁₆)-aralkyloxy-(C₁-C₁₀)-alkyl)-carbamoyl, CON(CH₂)ₕ, in which a CH₂ group can be replaced by, O, S, N—(C₁-C₈)-alkylimino, N—(C₃-C₈)-cycloalkylimino, N—(C₃-C₈)-cycloalkyl-(C₁-C₄)-alkylimino, N—(C₆-C₁₂)-arylimino, N—(C₇-C₁₆)-aralkylimino, N—(C₁-C₄)-alkoxy-(C₁-C₆)-alkylimino, and h is from 3 to 7; carbamoyloxy, N—(C₁-C₁₂)-alkylcarbamoyloxy, N,N-di-(C₁-C₁₂)-alkylcarbamoyloxy, N—(C₃-C₈)-cycloalkylcarbamoyloxy, N—(C₆-C₁₆)-arylcarbamoyloxy, N—(C₇-C₁₆)-aralkylcarbamoyloxy, N—(C₁-C₁₀)-alkyl-N—(C₆-C₁₂)-arylcarbamoyloxy, N—(C₁-C₁₀)-alkyl-N—(C₇-C₁₆)-aralkylcarbamoyloxy, N—((C₁-C₁₀)-alkyl)carbamoyloxy, N—((C₆-C₁₂)-aryloxy-(C₁-C₁₀)-alkyl)carbamoyloxy, N—((C₇-C₁₆)-aralkyloxy-(C₁-C₁₀)-alkyl)carbamoyloxy, N—(C₁-C₁₀)-alkyl-N—((C₁-C₁₀)-alkoxy-(C₁-C₁₀)-alkyl)carbamoyloxy, N—(C₁-C₁₀)-alkyl-N—((C₆-C₁₂)-aryloxy-(C₁-C₁₀)-alkyl)carbamoyloxy,N—(C₁-C₁₀)-alkyl-N—((C₇-C₁₆)-aralkyloxy-(C₁-C₁₀)-alkyl)carbamoyloxy, amino, (C₁-C₁₂)-alkylamino, di-(C₁-C₁₂)-alkylamino, (C₃-C₈)-cycloalkylamino, (C₃-C₁₂)-alkenylamino, (C₃-C₁₂)-alkynylamino, N—(C₆-C₁₂)-arylamino, N—(C₇-C₁₁)-aralkylamino, N-alkyl-aralkylamino, N-alkyl-arylamino, (C₁-C₁₂)-alkoxyamino, (C₁-C₁₂)-alkoxy-N—(C₁-C₁₀)-alkylamino, (C₁-C₁₂)-alkanoylamino, (C₃-C₈)-cycloalkanoylamino, (C₆-C₁₂)-aroylamino, (C₇-C₁₆)-aralkanoylamino, (C₁-C₁₂)-alkanoyl-N—(C₁-C₁₀)-alkylamino, (C₃-C₈)-cycloalkanoyl-N—(C₁-C₁₀)-alkylamino, (C₆-C₁₂)-aroyl-N—(C₁-C₁₀)-alkylamino, (C₇-C₁₁)-aralkanoyl-N—(C₁-C₁₀)-alkylamino, (C₁-C₁₂)-alkanoylamino-(C₁-C₈) -alkyl, (C₃-C₈)-cycloalkanoylamino-(C₁-C₈)-alkyl, (C₆-C₁₂)-aroylamino-(C₁-C₈)-alkyl, (C₇-C₁₆)-aralkanoylamino-(C₁-C₈)-alkyl, amino-(C₁-C₁₀)-alkyl, N—(C₁-C₁₀)-alkylamino-(C₁-C₁₀)-alkyl, N,N-di-(C₁-C₁₀)-alkylamino-(C₁-C₁₀)-alkyl, (C₃-C₈)-cycloalkylamino-(C₁-C₁₀)-alkyl, (C₁-C₁₂)-alkylmercapto, (C₁-C₁₂)-alkylsulfinyl, (C₁-C₁₂)-alkylsulfonyl, (C₆-C₁₆)-arylmercapto, (C₆-C₁₆)-arylsulfinyl, (C₆-C₁₆)-arylsulfonyl, (C₇-C₁₆)-aralkylmercapto, (C₇-C₁₆)-aralkylsulfinyl, or (C₇-C₁₆)-aralkylsulfonyl;
or wherein R¹ and R², or R² and R³ form a chain [CH₂]₀, which is saturated or unsaturated by a C═C double bond, in which 1 or 2 CH₂ groups are optionally replaced by O, S, SO, SO₂, or NR′, and R′ is hydrogen, (C₆-C₁₂)-aryl, (C₁-C₈)-alkyl, (C₁-C₈)-alkoxy-(C₁-C₈)-alkyl, (C₇-C₁₂)-aralkoxy-(C₁-C₈)-alkyl, (C₆-C₁₂)-aryloxy-(C₁-C₈)-alkyl, (C₁-C₁₀)-alkanoyl, optionally substituted (C₇-C₁₆)-aralkanoyl, or optionally substituted (C6-C12)-aroyl; and o is 3, 4 or 5;
or wherein the radicals R¹ and R², or R² and R³, together with the pyridine or pyridazine carrying them, form a 5,6,7,8-tetrahydroisoquinoline ring, a 5,6,7,8-tetrahydroquinoline ring, or a 5,6,7,8-tetrahydrocinnoline ring;
or wherein R¹ and R², or R² and R³ form a carbocyclic or heterocyclic 5- or 6-membered aromatic ring;
or where R¹ and R², or R² and R³, together with the pyridine or pyridazine carrying them, form an optionally substituted heterocyclic ring systems selected from thienopyridines, furanopyridines, pyridopyridines, pyrimidinopyridines, imidazopyridines, thiazolopyridines, oxazolopyridines, quinoline, isoquinoline, and cinnoline; where quinoline, isoquinoline or cinnoline preferably satisfy the formulae Ia, Ib and Ic: 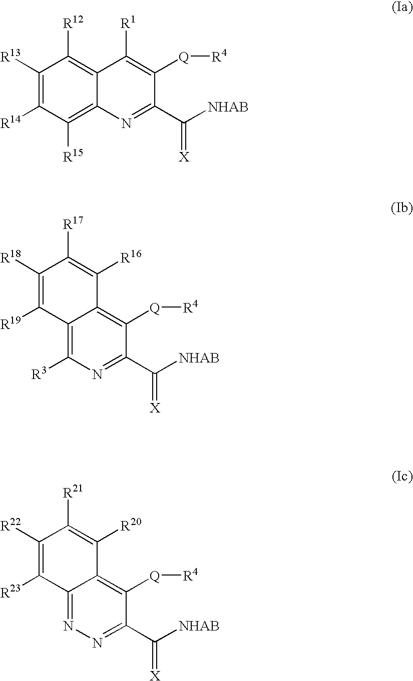
and the substituents R¹² to R²³ in each case independently of each other have the meaning of R¹, R² and R³;
or wherein the radicals R¹ and R², together with the pyridine carrying them, form a compound of Formula Id: 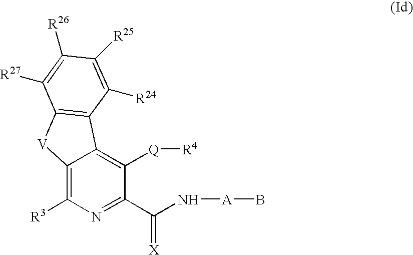
where V is S, O, or NR^{k}, and R^{k} is selected from hydrogen, (C₁-C₆)-alkyl, aryl, or benzyl; where an aryl radical may be optionally substituted by 1 to 5 substituents as defined above; and
R^{24,} R^{25,} R²⁶, and R²⁷ in each case independently of each other have the meaning of R¹, R² and R³;
f is 1 to 8;
g is 0 or 1 to (2f+1);
x is 0 to 3;
and h is 3 to 7;
including the physiologically active salts and prodrugs derived therefrom.

10. The method of claim 1, wherein the agent is selected from a compound of Formula (II): 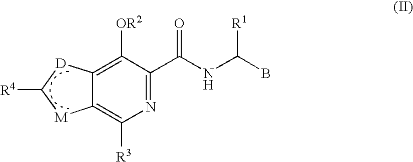
wherein
R¹ are selected from the group consisting of hydrogen, (C₁-C₆)-alkyl, (C₃-C₇)-cycloalkyl, aryl, or a substituent of the α-carbon atom of an α-amino acid, wherein the amino acid is a natural L-amino acid or its D-isomer;
B is —CO₂H or a CO₂-G carboxyl radical, where G is a radical of an alcohol G-OH in which G is selected from the group consisting of (C₁-C₂₀)-alkyl radical, (C₃-C₈) cycloalkyl radical, (C₂-C₂₀)-alkenyl radical, (C₃-C₈)-cycloalkenyl radical, retinyl radical, (C₂-C₂₀)-alkynyl radical, (C₄-C₂₀)-alkenynyl radical;
R² is selected from the group consisting of hydrogen, (C₁-C₁₀)-alkyl, (C₂-C₁₀)-alkenyl, (C₂-C₁₀)-alkynyl, wherein alkenyl or alkynyl contains one or two C—C multiple bonds; unsubstituted fluoroalkyl radical of the formula —[CH₂]ₓ—C_{f}H_{(2f+1-g)}—F_{g}, aryl, heteroaryl, and (C₇-C₁₁)-aralkyl;
one of D or M is —S—, and the other is ═C(R⁵)—;
R³, R⁴, and R⁵ are identical or different and are selected from the group consisting of hydrogen, hydroxyl, halogen, cyano, trifluoromethyl, nitro, carboxyl; (C₁-C₂₀)-alkyl, (C₃-C₈)-cycloalkyl, (C₃-C₈)-Cycloalkoxy, (C₆-C₁₂)-aryl, (C₇-C₁₆)-aralkyl, (C₇-C₁₆)-aralkenyl, (C₇-C₁₆)-aralkynyl, (C₂-C₂₀)-alkenyl, (C₂-C₂₀)-alkynyl, (C₁-C₂₀)-alkoxy, (C₂-C₂₀)-alkenyloxy, (C₂-C₂₀)-alkynyloxy, retinyloxy, (C₆-C₁₂)-aryloxy, (C₇-C₁₆)-aralkyloxy, (C₁-C₁₆)-hydroxyalkyl, —O—[CH₂]ₓC_{f}H_{(2f+1-g)}F_{g}, —OCF₂Cl, —OCF₂—CHFCl, (C₁-C₂₀)-alkylcarbonyl, (C₃-C₈)-cycloalkylcarbonyl, (C₆-C₁₂)-arylcarbonyl, (C₇-C₁₆)-aralkylcarbonyl, cinnamoyl, (C₂-C₂₀)-alkenylcarbonyl, (C₂-C₂₀)-alkynylcarbonyl, (C₁-C₂₀)-alkoxycarbonyl, (C₆-C₁₂)-aryloxycarbonyl, (C₇-C₁₆)-aralkyloxycarbonyl, (C₃-C₈)-cycloalkoxycarbonyl, (C₂-C₂₀)-alkenyloxycarbonyl, retinyloxycarbonyl, (C₂-C₂₀)-alkynyloxycarbonyl, (C₁-C₁₂)-alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N—(C₁-C₁₂)-alkylcarbamoyl, N,N-di-(C₁-C₁₂)-alkylcarbamoyl, N—(C₃-C₈)-cycloalkylcarbamoyl, N,N-dicyclo-(C₃-C₈)-alkylcarbamoyl, N—(C₁-C₁₀)-alkyl-N—(C₃-C₈)-cycloalkylcarbamoyl, N—((C₃-C₈)-cycloalkyl-(C₁-C₆)-alkyl)-carbamoyl, N-(+)-dehydroabietylcarbamoyl, N—(C₁-C₆)-alkyl-N-(+)-dehydroabietylcarbamoyl, N—(C₆-C₁₂)-arylcarbamoyl, N—(C₇-C₁₆)-aralkylcarbamoyl, N—(C₁-C₁₀)-alkyl-N—(C₆-C₁₆)-arylcarbamoyl, N—(C₁-C₁₀)-alkyl-N—(C₇-C₁₆)-aralkylcarbamoyl, carbamoyloxy, N—(C₁-C₁₂)-alkylcarbamoyloxy, N,N-di-(C₁-C₁₂)-alkylcarbamoyloxy, N—(C₃-C₈)-cycloalkylcarbamoyloxy, N—(C₆-C₁₂)-arylcarbamoyloxy, N—(C₇-C₁₆)-aralkylcarbamoyloxy, N—(C₁-C₁₀)-alkyl-N—(C₆-C₁₂)-arylcarbamoyloxy, N—(C₁-C₁₀)-alkyl-N—(C₇-C₁₆)-aralkylcarbamoyloxy, N—((C₁-C₁₀)-alkyl)-carbamoyloxy, N—(C₁-C₁₀)-alkyl-N—((C₇-C₁₆)-aralkyloxy-(C₁-C₁₀)-alkyl)-carbamoyloxyamino, (C₁-C₁₂)-alkylamino, di-(C₁-C₁₂)-alkylamino, (C₃-C₈)-cycloalkylamino, (C₃-C₁₂)-alkenylamino, (C₃-C₁₂)-alkynylamino, N—(C₆-C₁₂)-arylamino, N—(C₇-C₁₁)-aralkylamino, N-alkyl-aralkylamino, N-alkyl-arylamino, (C₁-C₁₂)-alkoxyamino, (C₁-C₁₂)-alkoxy-N—(C₁-C₁₀)-alkylamino, (C₁-C₁₂)-alkanoylamino, (C₃-C₈)-cycloalkanoylamino, (C₆-C₁₂)-aroylamino, (C₇-C₁₆)-aralkanoylamino, (C₁-C₁₂)-alkanoyl-N—(C₁-C₁₀)-alkylamino, (C₃-C₈)-cycloalkanoyl-N—(C₁-C₁₀)-alkylamino, (C₆-C₁₂)-aroyl-N—(C₁-C₁₀)-alkylamino, (C₇-C₁₁)-aralkanoyl-N—(C₁-C₁₀)-alkylamino, amino-(C₁-C₁₀)-alkyl, (C₁-C₂₀)-alkylmercapto, (C₁-C₂₀)-alkylsulfinyl, (C₁-C₂₀)-alkylsulfonyl, (C₆-C₁₂)-arylmercapto, (C₆-C₁₂)-arylsulfinyl, (C₆-C₁₂)-arylsulfonyl, (C₇-C₁₆)-aralkylmercapto, (C₇-C₁₆)-aralkylsulfinyl, (C₇-C₁₆)-aralkylsulfonyl, sulfamoyl, N—(C₁-C₁₀)-alkylsulfamoyl, N,N-di-(C₁-C₁₀)-alkylsulfamoyl, (C₃-C₈)-cycloalkylsulfamoyl, N—(C₆-C₁₂)-arylsulfamoyl, N—(C₇-C₁₆)-aralkylsulfamoyl, N—(C₁-C₁₀)-alkyl-N—(C₆-Cl₂)-arylsulfamoyl, N—(C₁-C₁₀)-alkyl-N—(C₇-C₁₆)-aralkylsulfamoyl, (C₁-C₁₀)-alkylsulfonamido, (C₇-C₁₆)-aralkylsulfonamido, and N—((C₁-C₁₀)-alkyl-(C₇-C₁₆)-aralkylsulfonamido; where an aryl radical may be substituted by 1 to 5 substituents selected from hydroxyl, halogen, cyano, trifluoromethyl, nitro, carboxyl, (C₂-C₁₆)-alkyl, (C₃-C₈)-cycloalkyl, (C₃-C₈)-cycloalkoxy, (C₆-C₁₂)-aryl, (C₇-C₁₆)-aralkyl, (C₂-C₁₆)-alkenyl, (C₂-C₁₂)-alkynyl, (C₁-C₁₆)-alkoxy, (C₁-C₁₆)-alkenyloxy, (C₆-C₁₂)-aryloxy, (C₇-C₁₆)-aralkyloxy, (C₁-C₈)-hydroxyalkyl, —O—[CH₂]ₓC_{f}H_{(2f+1-g)}F_{g}, —OCF₂Cl, and —OCF₂—CHFCl;
x is 0 to 3;
f is 1 to 8; and
g is 0 or 1 to (2f+1);
including the physiologically active salts and prodrugs derived therefrom.

11. The method of claim 1, wherein the agent is selected from a compound of Formula (III) 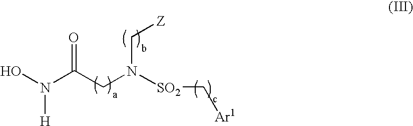
or pharmaceutically acceptable salts thereof, wherein:
a is an integer from 1 to 4;
b is an integer from 0 to 4;
c is an integer from 0 to 4;
Z is selected from the group consisting of (C₃-C₁₀) cycloalkyl, (C₃-C₁₀) cycloalkyl independently substituted with one or more Y¹, 3-10 membered heterocycloalkyl and 3-10 membered heterocycloalkyl independently substituted with one or more Y¹; (C₅-C₂₀) aryl, (C₅-C₂₀) aryl independently substituted with one or more Y¹, 5-20 membered heteroaryl and 5-20 membered heteroaryl independently substituted with one or more Y¹;
Ar¹ is selected from the group consisting of (C₅-C₂₀) aryl, (C₅-C₂₀) aryl independently substituted with one or more Y², 5-20 membered heteroaryl and 5-20 membered heteroaryl independently substituted with one or more Y²;
each Y¹ is independently selected from the group consisting of a lipophilic functional group, (C₅-C₂₀) aryl, (C₆-C₂₆) alkaryl, 5-20 membered heteroaryl and 6-26 membered alk-heteroaryl;
each Y² is independently selected from the group consisting of —R′, —O R′, —OR″, —SR′, —SR″, —NR′R′, —NO₂, —CN, -halogen, -trihalomethyl, trihalomethoxy, —C(O)R′, —C(O)OR′, —C(O)NR′R′, —C(O)NR′OR′, —C(NR′R′)═NOR′, —NR′—C(O)R′, —SO₂R′, —SO₂R″, —NR′—SO₂—R′, —NR′—C(O)—NR′R′, tetrazol-5-yl, —NR′—C(O)—OR′, —C(NR′R′)═NR′, —S(O)—R′, —S(O)R″, and —NR′—C(S)—NR′R′; and
each R′ is independently selected from the group consisting of —H, (C₁-C₈) alkyl, (C₂-C₈) alkenyl, and (C₂-C₈) alkynyl; and
each R″ is independently selected from the group consisting of (C₅-C₂₀) aryl and (C₅-C₂₀) aryl independently substituted with one or more —OR′, —SR′, —NR′R′, —NO₂, —CN, halogen or trihalomethyl groups,
or wherein c is 0 and Ar¹ is an N′ substituted urea-aryl, the compound has the structural formula (IIIa): 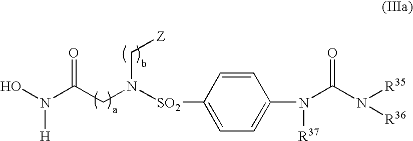
or pharmaceutically acceptable salts thereof, wherein:
a, b, and Z are as defined above; and
R³⁵ and R³⁶ are each independently selected from the group consisting of hydrogen, (C₁-C₈) alkyl, (C₂-C₈) alkenyl, (C₂-C₈) alkynyl, (C₃-C₁₀) cycloalkyl, (C₅-C₂₀) aryl, (C₅-C₂₀) substituted aryl, (C₆-C₂₆) alkaryl, (C₆-C₂₆) substituted alkaryl, 5-20 membered heteroaryl, 5-20 membered substituted heteroaryl, 6-26 membered alk-heteroaryl, and 6-26 membered substituted alk-heteroaryl; and
R³⁷ is independently selected from the group consisting of hydrogen, (C₁-C₈) alkyl, (C₂-C₈) alkenyl, and (C₂-C₈) alkynyl.

12. The method of claim 1, wherein the agent is selected from a compound of Formula (IV): 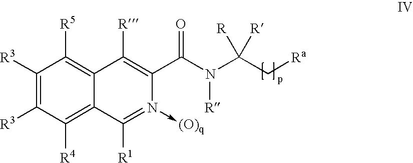
wherein:
q is zero or one;
p is zero or one;
R^{a} is —COOH or —WR⁸; provided that when R^{a} is —COOH, then p is zero, and when R^{a} is —WR⁸, then p is one;
W is selected from the group consisting of oxygen, —S(O)ₙ— and —NR⁹— where n is zero, one or two, R⁹ is selected from the group consisting of hydrogen, alkyl, substituted alkyl, acyl, aryl, substituted aryl, heteroaryl, substituted heteroaryl, heterocyclic and substituted heterocyclic and R⁸ is selected from the group consisting of hydrogen, alkyl, substituted alkyl, aryl, substituted aryl, heteroaryl, substituted heteroaryl, heterocyclic and substituted heterocyclic, or when W is —NR⁹— then R⁸ and R⁹, together with the nitrogen atom to which they are bound, can be joined to form a heterocyclic or a substituted heterocyclic group, provided that when W is —S(O)ₙ— and n is one or two, then R⁸ is not hydrogen;
R¹ is selected from the group consisting of hydrogen, alkyl, substituted alkyl, alkoxy, substituted alkoxy, amino, substituted amino, aminoacyl, aryl, substituted aryl, halo, heteroaryl, substituted heteroaryl, heterocyclic, substituted heterocyclic, and —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl or, when X is —NR⁷—, then R⁷ and R⁸, together with the nitrogen atom to which they are bound, can be joined to form a heterocyclic or substituted heterocyclic group;
R² and R³ are independently selected from the group consisting of hydrogen, alkyl, substituted alkyl, aryl, substituted aryl, heteroaryl, substituted heteroaryl, halo, hydroxy, cyano, —S(O)ₙ—N(R⁶)—R⁶ where n is 0, 1, or 2,—NR⁶C(O)NR⁶R⁶, —XR⁶ where X is oxygen, —S(O)ₙ— or —NR⁷— where n is zero, one or two, each R⁶ is independently selected from the group consisting of hydrogen, alkyl, substituted alkyl, aryl, substituted aryl, cycloalkyl, substituted cycloalkyl, heteroaryl, substituted heteroaryl, heterocyclic and substituted heterocyclic provided that when X is —SO— or —SO2—, then R⁶ is not hydrogen, and R⁷ is selected from the group consisting of hydrogen, alkyl, aryl, or R², R³ together with the carbon atom pendent thereto, form an aryl substituted aryl, heteroaryl, or substituted heteroaryl;
R⁴ and R⁵ are independently selected from the group consisting of hydrogen, halo, alkyl, substituted alkyl, alkoxy, substituted alkoxy, aryl, substituted aryl, heteroaryl, substituted heteroaryl and —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl or, when X is —NR⁷—, then R⁷ and R⁸, together with the nitrogen atom to which they are bound, can be joined to form a heterocyclic or substituted heterocyclic group;
R is selected from the group consisting of hydrogen, deuterium and methyl;
R′ is selected from the group consisting of hydrogen, deuterium, alkyl and substituted alkyl; alternatively, R and R′ and the carbon pendent thereto can be joined to form cycloalkyl, substituted cycloalkyl, heterocyclic or substituted heterocyclic group;
R″ is selected from the group consisting of hydrogen and alkyl or R″ together with R′ and the nitrogen pendent thereto can be joined to form a heterocyclic or substituted heterocyclic group;
R′″ is selected from the group consisting of hydroxy, alkoxy, substituted alkoxy, acyloxy, cycloalkoxy, substituted cycloalkoxy, aryloxy, substituted aryloxy, heteroaryloxy, substituted heteroaryloxy, aryl, —S(O)ₙ—R¹⁰ wherein R¹⁰ is selected from the group consisting of alkyl, substituted alkyl, cycloalkyl, substituted cycloalkyl, aryl, substituted aryl, heteroaryl and substituted heteroaryl and n is zero, one or two;
and pharmaceutically acceptable salts, esters and prodrugs thereof;

13. The method of claim 1, wherein the agent is selected from the group consisting of: [(1-Chloro-4-hydroxy-isoquinoline-3-carbonyl)-amino]-acetic acid, [(4-Hydroxy-1-methyl-7-phenoxy-isoquinoline-3-carbonyl)-amino]-acetic acid, {[4-Hydroxy-7-(4-methoxy-phenoxy)-isoquinoline-3-carbonyl]-amino}-acetic acid, [(4-Hydroxy-8-phenoxy-isoquinoline-3-carbonyl)-amino]-acetic acid, [(4-Hydroxy-1-methyl-8-phenoxy-isoquinoline-3-carbonyl)-amino]-acetic acid, [(7-Chloro-4-hydroxy-1-methyl-isoquinoline-3-carbonyl)-amino]-acetic acid, {[8-(4-Fluoro-phenoxy)-4-hydroxy-1-methyl-isoquinoline-3-carbonyl]-amino}-acetic acid, and [(4-cyano-7-hydroxy-thieno[3,2-c]pyridine-6-carbonyl)-amino]-acetic acid.

14. The method of claim 1, wherein the subject is a mammalian subject.

15. The method of claim 1, wherein the subject is a human subject.

16. The method of claim 1, wherein the subject is resistant or hyporesponsive to erythropoiesis stimulating protein treatment.

17. The method of claim 16, wherein the erythropoiesis stimulating protein treatment is recombinant human EPO treatment.

18. The method of claim 1, wherein the subject has previously been treated with recombinant human EPO therapy.

19. The method of claim 1, wherein the agent is used in conjunction with recombinant human EPO therapy.

20. The method of claim 1, wherein the agent is used in conjunction with iron supplement therapy

21. The method of claim 1, wherein the anemia is chemotherapy-induced anemia, anemia associated with chronic kidney disease, anemia of cancer, iron-deficiency anemia, and anemia of chronic disease.

22. The method of claim 1, wherein the subject has one or more risk factors for developing thrombosis.

23. The method of claim 1, wherein the agent is used in conjunction with an agent for reducing thrombosis.

24. The method claim 1, wherein the subject has one or more risk factors for developing hypertension.

25. The method of claim 1, wherein the agent is used in conjunction with an agent for reducing hypertension.

26. The method of claim 1, wherein the agent is administered orally, systemically, intravenously, or by injection.

27. The method of claim 1, wherein the agent is administered at a dose of 3 mg/kg, 6 mg/kg, 10 mg/kg, 15 mg/kg, 20 mg/kg or 30 mg/kg.

28. The method of claim 1, wherein the agent is administered two or three times weekly.

29. A pharmaceutical composition for treating anemia in a subject, wherein the anemia treatment is associated with a lower risk of thrombosis or hypertension compared to that observed with recombinant human EPO therapy, the composition comprising an effective amount of an agent that inhibits HIF hydroxylase activity.
